(19) United States
(12) Patent Application Publication
(10) Pub. No.: US 2023/0124760 A1
(43) Pub. Date: Apr. 20, 2023

(54) NASAL-SPRAY SAMPLING

(71) Applicant: HERO SCIENTIFC LTD., Jerusalem (IL)

(72) Inventor: Robert Eric LEVITZ, Beit Shemesh (IL)

(73) Assignee: HERO SCIENTIFC LTD., Jerusalem (IL)

(21) Appl. No.: 18/085,105

(22) Filed: Dec. 20, 2022

Related U.S. Application Data
(63) Continuation of application No. 17/980,200, filed on Nov. 3, 2022.
Application No. 17/980,200 is a continuation-in-part of application No. 17/921,672, filed on Jan. 1, 0001.
(60) Provisional application No. 63/020,723, filed on May 6, 2020.
(60) Provisional application No. 63/037,707, filed on Jun. 11, 2020.
(60) Provisional application No. 63/067,535, filed on Aug. 19, 2020.
(60) Provisional application No. 63/117,294, filed on Nov. 23, 2020.
(60) Provisional application No. 63/156,843, filed on Mar. 4, 2021.
(60) Provisional application No. 63/158,005, filed on Mar. 8, 2021.
(60) Provisional application No. 63/176,565, filed on Apr. 19, 2021.
(60) Provisional application No. 63/275,130, filed on Nov. 3, 2021.
(60) Provisional application No. 63/277,238, filed on Nov. 9, 2021.

(51) Int. Cl. A61B 10/00; G01N 1/40; A61M 11/00; G01N 33/569; A61M 3/02; A61H 35/04; A61M 15/08

(52) U.S. Cl. CPC A61B 10/0051; G01N 1/40; A61M 11/007; G01N 33/56983; A61M 3/0287; A61H 35/04; A61M 15/08; G01N 2001/4088; A61M 2202/206; A61M 2205/7545; A61H 2201/0157

(57) ABSTRACT

A method is provided that includes intranasally dispensing nasal wash fluid into a nasal cavity of a subject. Thereafter, a specimen sample is collected by performing an anterior nares nasal swab. The specimen sample is tested for the presence of a virus using a lateral flow immunoassay test strip. Other embodiments are also described.

### CROSS-REFERENCE TO RELATED APPLICATIONS

The present application is a continuation of U.S. application Ser. No. 17/980,200, filed Nov. 3, 2022, which:

(a) is a continuation-in-part of U.S. application Ser. No. 17/921,672, which is the US national stage of International Application PCT/IL2021/050519, filed May 6, 2021, which claims priority from U.S. Provisional Application 63/020,723, filed May 6, 2020; U.S. Provisional Application 63/037,707, filed Jun. 11, 2020; U.S. Provisional Application 63/067,535, filed Aug. 19, 2020; U.S. Provisional Application 63/117,294, filed Nov. 23, 2020; U.S. Provisional Application 63/156,843, filed Mar. 4, 2021; U.S. Provisional Application 63/158,005, filed Mar. 8, 2021; U.S. Provisional Application 63/166,378, filed Mar. 26, 2021; and U.S. Provisional Application 63/176,565, filed Apr. 19, 2021;

(b) claims the benefit of U.S. Provisional Application 63/275,130, filed Nov. 3, 2021; and

(c) claims the benefit of U.S. Provisional Application 63/277,238, filed Nov. 9, 2021.

All of the above-referenced applications are assigned to the assignee of the present application and incorporated herein by reference.

### FIELD OF THE APPLICATION

Applications of the present invention relate to collecting samples.

### BACKGROUND OF THE APPLICATION

Known sampling methods for collecting a respiratory specimen sample include nasopharyngeal swabbing to sample the posterior nasal passages and the nasopharynx, nasal washes and aspirations to sample deeply into the nasal cavity, and throat swabbing to sample the pharynx. These procedures are often uncomfortable for the patient, require a healthcare professional to perform the sampling procedure, and expose the healthcare worker and others to contaminating particles.

Patients can self-collect alternative sample types such as gargled fluid, nasal washes, and saliva. However, these sample types tend to decrease test sensitivity compared to swab samples and are thus are not currently in widespread use.

### SUMMARY OF THE APPLICATION

In some applications of the present invention, a method is provided that comprises intranasally dispensing nasal wash fluid into a nasal cavity of a subject. The nasal wash fluid generally loosens biological material in the nasal cavity. Thereafter, a specimen sample is collected by inserting a specimen sampler into the nasal cavity, typically such that a farthest-inserted portion of the specimen sampler is positioned in an anterior naris of the nasal cavity, and collecting a specimen sample on the specimen sampler by contacting (typically rubbing) a wall of the anterior naris with the specimen sampler. Alternatively or additionally, the specimen sample is collected by performing one or more nasal swabs selected from the group consisting of: an anterior nares swab and a mid-turbinate swab, typically without performing a nasopharyngeal swab.

In some applications of the present invention, a method is provided that comprises intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx. Thereafter, a specimen sample is collected that passed out of an anterior opening of an oral cavity of the subject and contains at least a portion of the biological material washed into the oropharynx by the nasal wash fluid. In some applications, collecting the specimen sample comprises expressing (e.g., spitting), by the subject, the specimen sample out of the anterior opening of the oral cavity. The specimen sample is typically collected into a collection receptacle.

Thereafter, information from extracellular vesicles present in the specimen sample is derived. As is known in the biological arts, extracellular vesicles are vesicles that are secreted into the extracellular space. Because extracellular vesicles are loaded with one or more of the following cellular components: cellular proteins, nucleic acids (such RNA and/or DNA components), nucleoproteins, modified lipids, polysaccharides, primary metabolites, and secondary metabolites, their use as biomarkers for cancer or pre-cancer screening and diagnosis of intracellular infections and other conditions (e.g., cardiovascular diseases, pregnancy disorders, and organ transplantation) has been explored.

Extracellular vesicles include exosomes, which are vesicles released by fusion of (a) multivesicular body(s) with a cell's plasma membrane, and are believed to play a role in cell-to-cell communication; microvesicles, which are vesicles released by budding directly from a cell's plasma membrane, and are believed to play a role in cell-to-cell communication; and apoptotic bodies, which are vesicles formed by the blebbing of a cell's plasma membrane during programmed cell death (apoptosis) and are often loaded with cellular organelles. For example, an overview of the application of exosomes as a liquid biopsy in clinical diagnosis is provided by Zhou B et al. in “Application of exosomes as liquid biopsy in clinical diagnosis,” Signal Transduction and Targeted Therapy volume 5, Article number: 144 (2020).

For some applications, deriving the information from the extracellular vesicles comprises identifying one or more proteins of the extracellular vesicles, one or more nucleic acids of the extracellular vesicles (such RNA and/or DNA components), one or more nucleoproteins of the extracellular vesicles, one or more modified lipids of the extracellular vesicles, one or more polysaccharides of the extracellular vesicles, one or more primary metabolites of the extracellular vesicles, and/or one or more secondary metabolites of the extracellular vesicles.

For some applications, the nasal-spray sampling collection techniques described herein are used for diagnosing and/or monitoring cancer, optionally even without deriving information from extracellular vesicles present in the specimen sample. The information may, for example, be derived by analyzing and/or detecting the presence of one or more cancer-related biomarkers, such as cancerous or pre-cancerous cells, immune cells, and/or cytokines indicative of a cancerous profile. Alternatively or additionally, deriving the information may comprise detecting HPV infection as a precursor to cancer. A final cancer diagnosis is typically only made upon performing additional testing, such as of tissue proliferation, e.g., based on a biopsy sample.

In some applications of the present invention, a method is provided that comprises collecting, from a subject, a liquid specimen sample that includes saliva and orally-dispensed oral wash fluid. The liquid specimen sample is passed through a filter to trap at least some of the saliva, the filter having a nominal pore size of between 30 microns and 1.5 mm. Information is derived from extracellular vesicles present in the saliva trapped by the filter in the saliva trapped by the filter.

There is therefore provided, in accordance with an Inventive Concept 1 of the present invention, a method including:

intranasally dispensing nasal wash fluid into a nasal cavity of a subject;

thereafter, collecting a specimen sample by performing an anterior nares nasal swab; and

testing the specimen sample for the presence of a virus using a lateral flow immunoassay test strip.

Inventive Concept 2. The method according to Inventive Concept 1, wherein collecting the specimen sample includes performing the anterior nares swab without performing a nasopharyngeal swab.
Inventive Concept 3. The method according to Inventive Concept 1, wherein collecting the specimen sample includes performing the anterior nares swab without performing a mid-turbinate swab.
Inventive Concept 4. The method according to Inventive Concept 1, wherein collecting the specimen sample includes performing the anterior nares swab without performing a mid-turbinate swab and without performing a nasopharyngeal swab.
Inventive Concept 5. The method according to Inventive Concept 1, wherein collecting the specimen sample includes collecting between 25 and 150 microliters of material on a single swab.
Inventive Concept 6. The method according to Inventive Concept 5, wherein collecting the specimen sample includes collecting between 25 and 125 microliters of material on the single swab.
Inventive Concept 7. The method according to Inventive Concept 5, wherein the single swab includes a flocked swab tip, and wherein collecting the specimen sample includes collecting between 25 and 150 microliters of the material on the flocked swab tip.

There is therefore provided, in accordance with an Inventive Concept 8 of the present invention, a method including:

intranasally dispensing nasal wash fluid into a nasal cavity of a subject;

thereafter, inserting a specimen sampler into the nasal cavity such that a farthest-inserted portion of the specimen sampler is positioned in an anterior naris of the nasal cavity;

collecting a specimen sample on the specimen sampler by rubbing a wall of the anterior naris with the specimen sampler; and

testing the specimen sample for the presence of a virus using a lateral flow immunoassay test strip.

Inventive Concept 9. The method according to Inventive Concept 8, wherein rubbing the wall of the anterior naris includes rotating the specimen sampler while rubbing the wall of the anterior naris with the specimen sampler.
Inventive Concept 10. The method according to Inventive Concept 8, wherein the specimen sampler includes a head including an absorbent material, and wherein rubbing the wall of the anterior naris with the specimen sampler includes rubbing the wall of the anterior naris with the absorbent material of the head of the specimen sampler.

There is further provided, in accordance with an Inventive Concept 11 of the present invention, a method including:

intranasally dispensing nasal wash fluid into a nasal cavity of a subject;

inserting a specimen sampler distally into the nasal cavity such that a farthest-inserted portion of the specimen sampler is positioned in an anterior naris of the nasal cavity;

collecting a specimen sample on the specimen sampler by contacting a wall of the anterior naris with the specimen sampler; and

testing the specimen sample for the presence of a virus using a lateral flow immunoassay test strip.

Inventive Concept 12. The method according to any one of Inventive Concepts 1-11, wherein intranasally dispensing the nasal wash fluid into the nasal cavity strengthens an intensity of a test band of the lateral flow immunoassay test strip when the virus is present in the anterior naris.
Inventive Concept 13. The method according to any one of Inventive Concepts 1-11, wherein intranasally dispensing the nasal wash fluid into the nasal cavity, when the virus is present in the anterior naris, strengthens an intensity of a test band of the lateral flow immunoassay test strip compared to collecting the specimen sample without intranasally dispensing the nasal wash fluid into the nasal cavity.
Inventive Concept 14. The method according to any one of Inventive Concepts 1-11, wherein the virus is Severe acute respiratory syndrome coronavirus 2 (SARS-CoV-2).
Inventive Concept 15. The method according to any one of Inventive Concepts 1-11, wherein the virus is an Influenza virus.
Inventive Concept 16. The method according to any one of Inventive Concepts 1-11, further including, after intranasally dispensing the nasal wash fluid and before collecting the specimen sample, sniffing, by the subject, the nasal wash fluid.
Inventive Concept 17. The method according to any one of Inventive Concepts 11-16, wherein the specimen sampler includes a head including an absorbent material, and wherein contacting the wall of the anterior naris with the specimen sampler includes contacting the wall of the anterior naris with the absorbent material of the head of the specimen sampler.
Inventive Concept 18. The method according to any one of Inventive Concepts 8-16, wherein the specimen sampler includes a swab.
Inventive Concept 19. The method according to Inventive Concept 18, wherein the swab includes a flocked swab tip.
Inventive Concept 20. The method according to Inventive Concept 18, wherein the swab includes a foam swab tip.
Inventive Concept 21. The method according to Inventive Concept 18, wherein the swab includes a tip having a greatest diameter of between 2 and 5 mm.
Inventive Concept 22. The method according to Inventive Concept 18, wherein the swab includes a tip having a collection surface area of between 200 and 300 mm2.
Inventive Concept 23. The method according to Inventive Concept 18, wherein the swab includes a tip having a volume of between 200 and 300 mm3.
Inventive Concept 24. The method according to Inventive Concept 18, wherein the swab includes a tip having a length of between 12 and 20 mm.
Inventive Concept 25. The method according to any one of Inventive Concepts 8-16, wherein the specimen sampler includes a brush.
Inventive Concept 26. The method according to any one of Inventive Concepts 8-16, wherein the specimen sampler includes a sponge.
Inventive Concept 27. The method according to any one of Inventive Concepts 8-16, wherein the specimen sampler includes a nasal plug.
Inventive Concept 28. The method according to any one of Inventive Concepts 8-16, wherein the specimen sampler is shaped so as not to define any nozzles.
Inventive Concept 29. The method according to any one of Inventive Concepts 8-16, wherein collecting the specimen sample includes collecting the specimen sample on the specimen sampler without using suction.
Inventive Concept 30. The method according to any one of Inventive Concepts 1-16, wherein intranasally dispensing includes intranasally dispensing, by the subject, the nasal wash fluid into the nasal cavity.
Inventive Concept 31. The method according to any one of Inventive Concepts 1-16, wherein collecting the specimen sample includes collecting the specimen sample by the subject.
Inventive Concept 32. The method according to any one of Inventive Concepts 1-16, further including waiting for a period of time between (a) concluding intranasally dispensing the nasal wash fluid into the nasal cavity and (b) collecting the specimen sample, the period of time at least 2 seconds and no more than 30 minutes.
Inventive Concept 33. The method according to any one of Inventive Concepts 1-16, wherein intranasally dispensing the nasal wash fluid includes intranasally dispensing the nasal wash fluid without blowing, by the subject, a nose of the subject immediately prior to intranasally dispensing the nasal wash fluid.
Inventive Concept 34. The method according to any one of Inventive Concepts 1-16, wherein intranasally dispensing the nasal wash fluid includes intranasally dispensing the nasal wash fluid into each of one or both nostrils of the subject in a total volume of between 0.15 and 0.5 mL per nostril.
Inventive Concept 35. The method according to any one of Inventive Concepts 8-16, wherein collecting the specimen sample on the specimen sampler includes collecting between 25 and 150 microliters of material on the specimen sampler.
Inventive Concept 36. The method according to Inventive Concept 35, wherein collecting the specimen sample on the specimen sampler includes collecting between 25 and 125 microliters of material on the specimen sampler.
Inventive Concept 37. The method according to Inventive Concept 35, wherein the specimen sampler includes a swab including a flocked swab tip, and wherein collecting the specimen sample on the specimen sampler includes collecting between 25 and 150 microliters of the material on the flocked swab tip.
Inventive Concept 38. The method according to any one of Inventive Concepts 1-16, further including, after intranasally dispensing the nasal wash fluid into the nasal cavity: orally dispensing oral wash fluid into an oral cavity of the subject via an anterior opening of the oral cavity; and gargling, by the subject, the oral wash fluid.
Inventive Concept 39. The method according to any one of Inventive Concepts 1-16, wherein intranasally dispensing the nasal wash fluid includes intranasally dispensing the nasal wash fluid as one or more types of fluid discharge selected from the group consisting of: a spray, a mist, a pressurized aerosol, steam, and an atomized fluid.
Inventive Concept 40. The method according to any one of Inventive Concepts 1-16, wherein intranasally dispensing the nasal wash fluid includes intranasally dispensing the nasal wash fluid as a fluid stream.

There is still further provided, in accordance with an Inventive Concept 41 of the present invention, a method including:

intranasally dispensing nasal wash fluid into a nasal cavity of a subject;

thereafter, collecting a specimen sample by performing an anterior nares nasal swab; and

testing the specimen sample for the presence of a virus using a lateral flow test strip.

Inventive Concept 42. The method according to Inventive Concept 41, wherein collecting the specimen sample includes performing the anterior nares swab without performing a nasopharyngeal swab.
Inventive Concept 43. The method according to Inventive Concept 41, wherein collecting the specimen sample includes performing the anterior nares swab without performing a mid-turbinate swab.
Inventive Concept 44. The method according to Inventive Concept 41, wherein collecting the specimen sample includes performing the anterior nares swab without performing a mid-turbinate swab and without performing a nasopharyngeal swab.
Inventive Concept 45. The method according to Inventive Concept 41, wherein collecting the specimen sample includes collecting between 25 and 150 microliters of material on a single swab.
Inventive Concept 46. The method according to Inventive Concept 45, wherein collecting the specimen sample includes collecting between 25 and 125 microliters of material on the single swab.
Inventive Concept 47. The method according to Inventive Concept 45, wherein the single swab includes a flocked swab tip, and wherein collecting the specimen sample includes collecting between 25 and 150 microliters of the material on the flocked swab tip.

There is additionally provided, in accordance with an Inventive Concept 48 of the present invention, a method including:

intranasally dispensing nasal wash fluid into a nasal cavity of a subject;

thereafter, inserting a specimen sampler into the nasal cavity such that a farthest-inserted portion of the specimen sampler is positioned in an anterior naris of the nasal cavity;

collecting a specimen sample on the specimen sampler by rubbing a wall of the anterior naris with the specimen sampler; and

testing the specimen sample for the presence of a virus using a lateral flow test strip.

Inventive Concept 49. The method according to Inventive Concept 48, wherein rubbing the wall of the anterior naris includes rotating the specimen sampler while rubbing the wall of the anterior naris with the specimen sampler.
Inventive Concept 50. The method according to Inventive Concept 48, wherein the specimen sampler includes a head including an absorbent material, and wherein rubbing the wall of the anterior naris with the specimen sampler includes rubbing the wall of the anterior naris with the absorbent material of the head of the specimen sampler.

There is yet additionally provided, in accordance with an Inventive Concept 51 of the present invention, a method including:

intranasally dispensing nasal wash fluid into a nasal cavity of a subject;

inserting a specimen sampler distally into the nasal cavity such that a farthest-inserted portion of the specimen sampler is positioned in an anterior naris of the nasal cavity;

collecting a specimen sample on the specimen sampler by contacting a wall of the anterior naris with the specimen sampler; and

testing the specimen sample for the presence of a virus using a lateral flow test strip.

Inventive Concept 52. The method according to any one of Inventive Concepts 41-51, wherein testing the specimen sample for the presence of the virus includes performing a molecular test that utilizes polymerase chain reaction (PCR) in combination with the lateral flow test strip.
Inventive Concept 53. The method according to any one of Inventive Concepts 41-51, wherein the lateral flow test strip is a CRISPR-based lateral flow test strip, and wherein testing the specimen sample for the presence of the virus includes testing the specimen sample for the presence of the virus using the CRISPR-based lateral flow test strip.
Inventive Concept 54. The method according to Inventive Concept 53, wherein the CRISPR-based lateral flow test strip is a CRISPR/Cas9-based lateral flow test strip, and wherein testing the specimen sample for the presence of the virus includes testing the specimen sample for the presence of the virus using the CRISPR/Cas9-based lateral flow test strip.
Inventive Concept 55. The method according to any one of Inventive Concepts 41-51, wherein intranasally dispensing the nasal wash fluid into the nasal cavity strengthens an intensity of a test band of the lateral flow test strip when the virus is present in the anterior naris.
Inventive Concept 56. The method according to any one of Inventive Concepts 41-51, wherein intranasally dispensing the nasal wash fluid into the nasal cavity, when the virus is present in the anterior naris, strengthens an intensity of a test band of the lateral flow test strip compared to collecting the specimen sample without intranasally dispensing the nasal wash fluid into the nasal cavity.
Inventive Concept 57. The method according to any one of Inventive Concepts 41-51, wherein the virus is Severe acute respiratory syndrome coronavirus 2 (SARS-CoV-2).
Inventive Concept 58. The method according to any one of Inventive Concepts 41-51, wherein the virus is an Influenza virus.
Inventive Concept 59. The method according to any one of Inventive Concepts 41-51, further including, after intranasally dispensing the nasal wash fluid and before collecting the specimen sample, sniffing, by the subject, the nasal wash fluid.
Inventive Concept 60. The method according to any one of Inventive Concepts 11-59, wherein the specimen sampler includes a head including an absorbent material, and wherein contacting the wall of the anterior naris with the specimen sampler includes contacting the wall of the anterior naris with the absorbent material of the head of the specimen sampler.
Inventive Concept 61. The method according to any one of Inventive Concepts 48-59, wherein the specimen sampler includes a swab.
Inventive Concept 62. The method according to Inventive Concept 61, wherein the swab includes a flocked swab tip.
Inventive Concept 63. The method according to Inventive Concept 61, wherein the swab includes a foam swab tip.
Inventive Concept 64. The method according to Inventive Concept 61, wherein the swab includes a tip having a greatest diameter of between 2 and 5 mm.
Inventive Concept 65. The method according to Inventive Concept 61, wherein the swab includes a tip having a collection surface area of between 200 and 300 mm2.
Inventive Concept 66. The method according to Inventive Concept 61, wherein the swab includes a tip having a volume of between 200 and 300 mm3.
Inventive Concept 67. The method according to Inventive Concept 61, wherein the swab includes a tip having a length of between 12 and 20 mm.
Inventive Concept 68. The method according to any one of Inventive Concepts 48-59, wherein the specimen sampler includes a brush.
Inventive Concept 69. The method according to any one of Inventive Concepts 48-59, wherein the specimen sampler includes a sponge.
Inventive Concept 70. The method according to any one of Inventive Concepts 48-59, wherein the specimen sampler includes a nasal plug.
Inventive Concept 71. The method according to any one of Inventive Concepts 48-59, wherein the specimen sampler is shaped so as not to define any nozzles.
Inventive Concept 72. The method according to any one of Inventive Concepts 48-59, wherein collecting the specimen sample includes collecting the specimen sample on the specimen sampler without using suction.
Inventive Concept 73. The method according to any one of Inventive Concepts 41-59, wherein intranasally dispensing includes intranasally dispensing, by the subject, the nasal wash fluid into the nasal cavity.
Inventive Concept 74. The method according to any one of Inventive Concepts 41-59, wherein collecting the specimen sample includes collecting the specimen sample by the subject.
Inventive Concept 75. The method according to any one of Inventive Concepts 41-59, further including waiting for a period of time between (a) concluding intranasally dispensing the nasal wash fluid into the nasal cavity and (b) collecting the specimen sample, the period of time at least 2 seconds and no more than 30 minutes.
Inventive Concept 76. The method according to any one of Inventive Concepts 41-59, wherein intranasally dispensing the nasal wash fluid includes intranasally dispensing the nasal wash fluid without blowing, by the subject, a nose of the subject immediately prior to intranasally dispensing the nasal wash fluid.
Inventive Concept 77. The method according to any one of Inventive Concepts 41-59, wherein intranasally dispensing the nasal wash fluid includes intranasally dispensing the nasal wash fluid into each of one or both nostrils of the subject in a total volume of between 0.15 and 0.5 mL per nostril.
Inventive Concept 78. The method according to any one of Inventive Concepts 48-59, wherein collecting the specimen sample on the specimen sampler includes collecting between 25 and 150 microliters of material on the specimen sampler.
Inventive Concept 79. The method according to Inventive Concept 78, wherein collecting the specimen sample on the specimen sampler includes collecting between 25 and 125 microliters of material on the specimen sampler.
Inventive Concept 80. The method according to Inventive Concept 78, wherein the specimen sampler includes a swab including a flocked swab tip, and wherein collecting the specimen sample on the specimen sampler includes collecting between 25 and 150 microliters of the material on the flocked swab tip.
Inventive Concept 81. The method according to any one of Inventive Concepts 41-59, wherein intranasally dispensing the nasal wash fluid includes intranasally dispensing the nasal wash fluid as one or more types of fluid discharge selected from the group consisting of: a spray, a mist, a pressurized aerosol, steam, and an atomized fluid.
Inventive Concept 82. The method according to any one of Inventive Concepts 41-59, wherein intranasally dispensing the nasal wash fluid includes intranasally dispensing the nasal wash fluid as a fluid stream.

There is also provided, in accordance with an Inventive Concept 83 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx;

thereafter, collecting a specimen sample that passed out of an anterior opening of an oral cavity of the subject and contains at least a portion of the biological material washed into the oropharynx by the nasal wash fluid; and

thereafter, deriving information from extracellular vesicles present in the specimen sample.

Inventive Concept 84. The method according to Inventive Concept 83, wherein deriving the information includes performing an assay to derive the information.
Inventive Concept 85. The method according to Inventive Concept 83, wherein deriving the information includes ascertaining whether the extracellular vesicles present in the specimen sample include a biological marker.
Inventive Concept 86. The method according to Inventive Concept 83, wherein the extracellular vesicles are exosomes, and wherein deriving the information includes deriving the information from the exosomes present in the specimen sample.
Inventive Concept 87. The method according to Inventive Concept 83, wherein the extracellular vesicles are microvesicles, and wherein deriving the information includes deriving the information from the microvesicles present in the specimen sample.
Inventive Concept 88. The method according to Inventive Concept 83, wherein the extracellular vesicles are apoptotic bodies, and wherein deriving the information includes deriving the information from the apoptotic bodies present in the specimen sample.
Inventive Concept 89. The method according to Inventive Concept 83, wherein deriving the information from the extracellular vesicles includes identifying one or more proteins of the extracellular vesicles.
Inventive Concept 90. The method according to Inventive Concept 83, wherein deriving the information from the extracellular vesicles includes identifying one or more nucleic acids of the extracellular vesicles.
Inventive Concept 91. The method according to Inventive Concept 83, wherein collecting the specimen sample includes collecting the specimen sample into a collection receptacle.
Inventive Concept 92. The method according to Inventive Concept 83, wherein intranasally dispensing includes intranasally dispensing, by the subject, the nasal wash fluid into the nasal cavity.
Inventive Concept 93. The method according to Inventive Concept 83, further including, before collecting the specimen sample, tilting back, by the subject, a head of the subject.
Inventive Concept 94. The method according to Inventive Concept 83, further including, while intranasally dispensing the nasal wash fluid, tilting back, by the subject, a head of the subject.
Inventive Concept 95. The method according to Inventive Concept 83, further including, before intranasally dispensing the nasal wash fluid, tilting back, by the subject, a head of the subject.
Inventive Concept 96. The method according to Inventive Concept 83, further including, after intranasally dispensing the nasal wash fluid, tilting a head of the subject farther back than while intranasally dispensing the nasal wash fluid and farther back than a neutral position.
Inventive Concept 97. The method according to Inventive Concept 83, wherein intranasally dispensing the nasal wash fluid includes intranasally dispensing the nasal wash fluid into the nasal cavity while a head of the subject is held in a neutral position or a tilted-forward position.
Inventive Concept 98. The method according to Inventive Concept 83, further including waiting for a period of time between (a) concluding intranasally dispensing the nasal wash fluid into the nasal cavity and (b) collecting the specimen sample, the period of time at least 5 minutes and less than 90 minutes.
Inventive Concept 99. The method according to Inventive Concept 83, wherein intranasally dispensing the nasal wash fluid includes intranasally dispensing the nasal wash fluid as one or more types of fluid discharge selected from the group consisting of: a fluid stream, a spray, a mist, a pressurized aerosol, and an atomized fluid.
Inventive Concept 100. The method according to Inventive Concept 83, wherein intranasally dispensing the nasal wash fluid includes intranasally dispensing the nasal wash fluid as steam.
Inventive Concept 101. The method according to Inventive Concept 83, wherein intranasally dispensing the nasal wash fluid includes intranasally dispensing the nasal wash fluid in a circular rotary motion to generate dispersion within the nasal cavity.
Inventive Concept 102. The method according to Inventive Concept 83, wherein intranasally dispensing the nasal wash fluid into the nasal cavity includes intranasally dispensing a total volume of 0.2-20 mL of the nasal wash fluid into the nasal cavity.
Inventive Concept 103. The method according to Inventive Concept 102, wherein intranasally dispensing the nasal wash fluid into the nasal cavity includes intranasally dispensing a total volume of 0.6-20 mL of the nasal wash fluid into the nasal cavity.
Inventive Concept 104. The method according to Inventive Concept 102, wherein intranasally dispensing the nasal wash fluid into the nasal cavity includes intranasally dispensing a total volume of 0.2-10 mL of the nasal wash fluid into the nasal cavity.
Inventive Concept 105. The method according to Inventive Concept 104, wherein intranasally dispensing the nasal wash fluid into the nasal cavity includes intranasally dispensing a total volume of 0.2-5 mL of the nasal wash fluid into the nasal cavity.
Inventive Concept 106. The method according to Inventive Concept 105, wherein intranasally dispensing the nasal wash fluid into the nasal cavity includes intranasally dispensing a total volume of 0.6-5 mL of the nasal wash fluid into the nasal cavity.
Inventive Concept 107. The method according to Inventive Concept 102, wherein intranasally dispensing the nasal wash fluid into the nasal cavity includes intranasally dispensing the total volume of the nasal wash fluid in a plurality of pulses.
Inventive Concept 108. The method according to Inventive Concept 107, wherein each of the pulses has a volume of 0.02-10 mL.
Inventive Concept 109. The method according to Inventive Concept 108, wherein each of the pulses has a volume of 0.02-1 mL.
Inventive Concept 110. The method according to Inventive Concept 83, wherein intranasally dispensing the nasal wash fluid into the nasal cavity includes intranasally dispensing the nasal wash fluid with a sprayed particle size of 5-50 microns.
Inventive Concept 111. The method according to Inventive Concept 110, wherein intranasally dispensing the nasal wash fluid includes intranasally dispensing the nasal wash fluid with a sprayed particle size of 5-15 microns.
Inventive Concept 112. The method according to Inventive Concept 110, wherein intranasally dispensing the nasal wash fluid includes intranasally dispensing the nasal wash fluid with a sprayed particle size of 15-20 microns.
Inventive Concept 113. The method according to Inventive Concept 83, wherein intranasally dispensing the nasal wash fluid into the nasal cavity includes intranasally dispensing the nasal wash fluid into the nasal cavity while the subject is lying supine.
Inventive Concept 114. The method according to any one of Inventive Concepts 83-113, wherein collecting the specimen sample includes expressing, by the subject, the specimen sample out of the oropharynx via the anterior opening of the oral cavity.
Inventive Concept 115. The method according to Inventive Concept 114, wherein expressing the specimen sample includes spitting, by the subject, the specimen sample out of the oropharynx via the anterior opening of the oral cavity.
Inventive Concept 116. The method according to Inventive Concept 114, wherein expressing the specimen sample includes expressing the specimen sample out of the oropharynx via the anterior opening of the oral cavity after sensing, by the subject, the nasal wash fluid in the oropharynx.
Inventive Concept 117. The method according to Inventive Concept 116, wherein expressing the specimen sample includes expressing the specimen sample out of the oropharynx via the anterior opening of the oral cavity after tasting, by the subject, the nasal wash fluid in the oropharynx.
Inventive Concept 118. The method according to Inventive Concept 117, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 119. The method according to Inventive Concept 114, wherein intranasally dispensing includes intranasally dispensing, by a healthcare worker, the nasal wash fluid into the nasal cavity.
Inventive Concept 120. The method according to any one of Inventive Concepts 83-113, wherein collecting the specimen sample includes drawing the specimen sample out of the oropharynx via the anterior opening of the oral cavity using an absorbent material, without swabbing the oral cavity or the oropharynx.
Inventive Concept 121. The method according to any one of Inventive Concepts 83-113, wherein intranasally dispensing includes intranasally dispensing, by a healthcare worker, the nasal wash fluid into the nasal cavity.
Inventive Concept 122. The method according to Inventive Concept 121, wherein collecting the specimen sample includes receiving the specimen sample by a healthcare worker from the subject.
Inventive Concept 123. The method according to Inventive Concept 122, wherein receiving the specimen sample includes receiving the specimen sample that was expressed out of the oropharynx via the anterior opening of the oral cavity.
Inventive Concept 124. The method according to Inventive Concept 123, wherein receiving the specimen sample includes receiving the specimen sample that was spit out of the oropharynx via the anterior opening of the oral cavity.
Inventive Concept 125. The method according to Inventive Concept 121, wherein collecting the specimen sample includes aspirating the specimen sample out of the oropharynx via the anterior opening of the oral cavity.
Inventive Concept 126. The method according to Inventive Concept 121, wherein collecting the specimen sample includes instructing the subject, by the healthcare worker, to express the specimen sample out of the oropharynx via the anterior opening of the oral cavity.
Inventive Concept 127. The method according to Inventive Concept 126, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 128. The method according to any one of Inventive Concepts 83-113, further including, after intranasally dispensing the nasal wash fluid and before collecting the specimen sample, sniffing, by the subject, the biological material with the nasal wash fluid from the nasal cavity posteriorly toward the nasopharynx.
Inventive Concept 129. The method according to any one of Inventive Concepts 83-113, further including, after intranasally dispensing the nasal wash fluid and before collecting the specimen sample, snorting, by the subject, the biological material with the nasal wash fluid from the nasal cavity toward the nasopharynx.
Inventive Concept 130. The method according to any one of Inventive Concepts 83-113, further including diagnosing a condition based at least in part on the information.
Inventive Concept 131. The method according to Inventive Concept 130, wherein diagnosing the condition includes diagnosing cancer based at least in part on the information.
Inventive Concept 132. The method according to Inventive Concept 131, wherein diagnosing the cancer includes screening the specimen sample for cells that are possibly cancerous or precancerous.
Inventive Concept 133. The method according to any one of Inventive Concepts 83-113, further including monitoring a condition using the information.
Inventive Concept 134. The method according to Inventive Concept 133, wherein monitoring the condition includes monitoring cancer.
Inventive Concept 135. The method according to any one of Inventive Concepts 83-113, wherein deriving the information from the extracellular vesicles present in the specimen sample includes concentrating the specimen sample by filtration prior to deriving the information, by passing the specimen sample through one or more filters.
Inventive Concept 136. The method according to any one of Inventive Concepts 83-113, wherein intranasally dispensing the nasal wash fluid into the nasal cavity includes intranasally dispensing the nasal wash fluid unidirectionally within the nasal cavity.
Inventive Concept 137. The method according to Inventive Concept 136, wherein intranasally dispensing the nasal wash fluid unidirectionally within the nasal cavity includes intranasally dispensing the nasal wash fluid unidirectionally within the nasal cavity in a superior direction.
Inventive Concept 138. The method according to Inventive Concept 136, wherein intranasally dispensing the nasal wash fluid unidirectionally within the nasal cavity includes intranasally dispensing the nasal wash fluid unidirectionally within the nasal cavity in a superoposterior direction.
Inventive Concept 139. The method according to Inventive Concept 138, wherein intranasally dispensing the nasal wash fluid unidirectionally within the nasal cavity in a superoposterior direction includes intranasally dispensing the nasal wash fluid unidirectionally within the nasal cavity toward an olfactory area of the nasal cavity.
Inventive Concept 140. The method according to Inventive Concept 136, wherein intranasally dispensing the nasal wash fluid unidirectionally within the nasal cavity includes intranasally dispensing the nasal wash fluid unidirectionally within the nasal cavity in a posterior direction.
Inventive Concept 141. The method according to Inventive Concept 140, wherein intranasally dispensing the nasal wash fluid unidirectionally within the nasal cavity in a posterior direction includes intranasally dispensing the nasal wash fluid unidirectionally within the nasal cavity toward the nasopharynx.
Inventive Concept 142. The method according to any one of Inventive Concepts 83-113, wherein intranasally dispensing the nasal wash fluid into the nasal cavity includes intranasally dispensing the nasal wash fluid multidirectionally within the nasal cavity.
Inventive Concept 143. The method according to Inventive Concept 142, wherein intranasally dispensing the nasal wash fluid multidirectionally within the nasal cavity includes dispensing the nasal wash fluid into the nasal cavity:

in a posterior direction toward the nasopharynx, and

in a superoposterior direction toward an olfactory area of the nasal cavity.

Inventive Concept 144. The method according to Inventive Concept 143, wherein intranasally dispensing the nasal wash fluid multidirectionally within the nasal cavity includes dispensing the nasal wash fluid into the nasal cavity additionally in at least a superior direction toward an inferior border of upper lateral cartilage of the nasal cavity.
Inventive Concept 145. The method according to Inventive Concept 144, wherein intranasally dispensing the nasal wash fluid multidirectionally within the nasal cavity includes dispensing the nasal wash fluid into the nasal cavity additionally bilaterally toward a septum and a lateral wall of the nasal cavity.
Inventive Concept 146. The method according to Inventive Concept 142, wherein intranasally dispensing the nasal wash fluid multidirectionally within the nasal cavity includes intranasally dispensing the nasal wash fluid via a plurality of nozzles.
Inventive Concept 147. The method according to Inventive Concept 146, wherein intranasally dispensing the nasal wash fluid multidirectionally within the nasal cavity via the plurality of nozzles includes intranasally dispensing the nasal wash fluid into the nasal cavity:

in a posterior direction toward the nasopharynx via a first subset of one or more of the plurality of nozzles, and in a superoposterior direction toward an olfactory area of the nasal cavity via a second subset of one or more of the plurality of nozzles, the first and the second subsets not including any nozzles common to both subsets.

Inventive Concept 148. The method according to Inventive Concept 147,

wherein the plurality of nozzles includes only first and second nozzles, and wherein the first and the second subsets consist of the first and the second nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle, and
in the superoposterior direction toward the olfactory area of the nasal cavity via the second nozzle.
   Inventive Concept 149. The method according to Inventive Concept 147, wherein intranasally dispensing the nasal wash fluid multidirectionally within the nasal cavity via the plurality of nozzles includes intranasally dispensing the nasal wash fluid into the nasal cavity:

in the posterior direction toward the nasopharynx via the first subset of one or more of the plurality of nozzles,

in the superoposterior direction toward the olfactory area of the nasal cavity via the second subset of one or more of the plurality of nozzles, and

at least in a superior direction toward an inferior border of upper lateral cartilage of the nasal cavity via a third subset of one or more of the plurality of nozzles, wherein none of the nozzles is included in more than one of the first, the second, and the third subsets.

Inventive Concept 150. The method according to Inventive Concept 149, wherein intranasally dispensing the nasal wash fluid in at least the superior direction toward the superior wall includes dispensing the nasal wash fluid into the nasal cavity additionally bilaterally toward a septum and a lateral wall of the nasal cavity via the third subset of one or more of the plurality of nozzles.
Inventive Concept 151. The method according to Inventive Concept 149,

wherein the plurality of nozzles includes only first, second, and third nozzles, and wherein the first, the second, and the third subsets consist of the first, the second, and the third nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle,
in the superoposterior direction toward the olfactory area of the nasal cavity via the second nozzle, and
at least in the superior direction toward an inferior border of upper lateral cartilage of the nasal cavity via the third nozzle.
   Inventive Concept 152. The method according to Inventive Concept 146, wherein intranasally dispensing the nasal wash fluid multidirectionally within the nasal cavity includes intranasally dispensing the nasal wash fluid within the nasal cavity via the plurality of nozzles with respective different fluid flow characteristics, the fluid flow characteristics including one or more characteristics selected from the group consisting of: volume, sprayed particle size, velocity, and flow rate.
   Inventive Concept 153. The method according to any one of Inventive Concepts 83-113, wherein intranasally dispensing the nasal wash fluid into the nasal cavity includes intranasally dispensing nasal wash fluid via both nostrils of the subject simultaneously.
   Inventive Concept 154. The method according to any one of Inventive Concepts 83-113, wherein the nasal wash fluid does not include a drug molecule.
   Inventive Concept 155. The method according to any one of Inventive Concepts 83-113, wherein the nasal wash fluid includes a non-irritant solution.
   Inventive Concept 156. The method according to Inventive Concept 155, wherein the non-irritant solution consists of water.
   Inventive Concept 157. The method according to Inventive Concept 155, wherein the non-irritant solution includes saline solution.
   Inventive Concept 158. The method according to Inventive Concept 157, wherein the non-irritant saline solution includes phosphate-buffered saline solution.
   Inventive Concept 159. The method according to any one of Inventive Concepts 83-113, wherein the nasal wash fluid includes a tastant.
   Inventive Concept 160. The method according to any one of Inventive Concepts 83-113, wherein the nasal wash fluid includes a color additive, and collecting the specimen sample includes confirming that the specimen sample contains at least a portion of the nasal wash fluid by visually ascertaining that the specimen sample includes the color additive.
   Inventive Concept 161. The method according to any one of Inventive Concepts 83-113, wherein the nasal wash fluid includes a chemical additive, and collecting the specimen sample includes confirming that the specimen sample contains at least a portion of the nasal wash fluid by ascertaining that the specimen sample includes the chemical additive.
   Inventive Concept 162. The method according to any one of Inventive Concepts 83-113, further including, before collecting the specimen sample, intranasally dispensing a vapor into the nasal cavity to loosen the biological material in the nasal cavity.
   Inventive Concept 163. The method according to Inventive Concept 162, wherein intranasally dispensing the vapor includes inhaling, by the subject, the vapor into the nasal cavity.
   Inventive Concept 164. The method according to any one of Inventive Concepts 83-113, further including, after intranasally dispensing and before collecting the specimen sample, swishing in the oral cavity, by the subject, the nasal wash fluid washed into the oropharynx.
   Inventive Concept 165. The method according to any one of Inventive Concepts 83-113, further including, after intranasally dispensing and before collecting the specimen sample, gargling, by the subject, the nasal wash fluid washed into the oropharynx.
   Inventive Concept 166. The method according to Inventive Concept 165, wherein the method further includes, after gargling the nasal wash fluid and before collecting the specimen sample, while holding the nasal wash fluid in the oropharynx, sniffing an additional portion of the nasal wash fluid from the nasal cavity toward the oropharynx such that the additional portion of the nasal wash fluid is added to the nasal wash fluid already in the oropharynx.
   Inventive Concept 167. The method according to Inventive Concept 166, wherein the method further includes, after sniffing the additional portion of the nasal wash fluid from the nasal cavity toward the oropharynx, again gargling the nasal wash fluid washed into the oropharynx.
   Inventive Concept 168. The method according to any one of Inventive Concepts 83-113,

wherein the method further includes, before collecting the specimen sample:
orally dispensing oral wash fluid into the oral cavity via the anterior opening of the oral cavity; and
swishing, by the subject, the oral wash fluid in the oral cavity, and

wherein collecting includes collecting the specimen sample that passed out of the anterior opening of the oral cavity and contains at least a portion of the oral wash fluid and the at least a portion of the biological material washed into the oropharynx with the nasal wash fluid.

Inventive Concept 169. The method according to any one of Inventive Concepts 83-113,

wherein the method further includes, before collecting the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein collecting includes collecting the specimen sample that passed out of the anterior opening of the oral cavity and contains at least a portion of the mixture and the at least a portion of the biological material washed into the oropharynx with the nasal wash fluid.

Inventive Concept 170. The method according to any one of Inventive Concepts 83-113,

wherein the method further includes, before collecting the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein collecting includes collecting the specimen sample that passed out of the anterior opening of the oral cavity and contains at least a portion of the mixture and the at least a portion of the biological material washed into the oropharynx with the nasal wash fluid.

Inventive Concept 171. The method according to any one of Inventive Concepts 83-113,

wherein the method further includes, before collecting the specimen sample:
orally dispensing oral wash fluid into the oral cavity via the anterior opening of the oral cavity; and
gargling, by the subject, the oral wash fluid, and

wherein collecting includes collecting the specimen sample that passed out of the anterior opening of the oral cavity and contains at least a portion of the oral wash fluid and the at least a portion of the biological material washed into the oropharynx with the nasal wash fluid.

Inventive Concept 172. The method according to any one of Inventive Concepts 169 and 170, wherein orally dispensing the oral wash fluid into the oral cavity includes orally dispensing the oral wash fluid into the oral cavity prior to intranasally dispensing the nasal wash fluid into the nasal cavity.
Inventive Concept 173. The method according to any one of Inventive Concepts 169 and 170, wherein orally dispensing the oral wash fluid into the oral cavity includes orally dispensing the oral wash fluid into the oral cavity after intranasally dispensing the nasal wash fluid into the nasal cavity.
Inventive Concept 174. The method according to Inventive Concept 173, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 175. The method according to any one of Inventive Concepts 169, 170, and 171,

wherein the specimen sample is a first specimen sample, and the mixture is a first mixture,

wherein the method further includes, after collecting the first specimen sample:
waiting for a period of time, the period of time at least 5 minutes and less than 90 minutes;
thereafter, orally dispensing additional oral wash fluid into the oral cavity via the anterior opening of the oral cavity;
thereafter, gargling, by the subject; and
thereafter, collecting a second specimen sample that passed out of the anterior opening of the oral cavity and contains at least a portion of the additional oral wash fluid and the at least a portion of the biological material washed into the oropharynx with the nasal wash fluid, and

wherein the method does not include intranasally dispensing nasal wash fluid into the nasal cavity after collecting the first specimen sample.

Inventive Concept 176. The method according to any one of Inventive Concepts 169, 170, and 171,

wherein the nasal wash fluid includes a first color additive,

wherein the oral wash fluid includes a second colored additive, the first and the second color additives having different colors, and

wherein collecting the specimen sample includes confirming that the specimen sample contains at least a portion of the nasal wash fluid and at least a portion of the oral wash fluid by visually ascertaining that the specimen sample has a color produced by a combination of the first and the second color additives.

Inventive Concept 177. The method according to any one of Inventive Concepts 169, 170, and 171, wherein the oral wash fluid includes a non-irritant solution.
Inventive Concept 178. The method according to Inventive Concept 177, wherein the non-irritant solution consists of water.
Inventive Concept 179. The method according to Inventive Concept 177, wherein the non-irritant solution includes a tastant.
Inventive Concept 180. The method according to Inventive Concept 177, wherein the non-irritant solution includes saline solution.
Inventive Concept 181. The method according to Inventive Concept 180, wherein the non-irritant saline solution includes phosphate-buffered saline solution.
Inventive Concept 182. The method according to any one of Inventive Concepts 169 and 170, wherein the method further includes, after gargling the mixture and before collecting the specimen sample, while holding the mixture in the oropharynx, sniffing an additional portion of the nasal wash fluid from the nasal cavity toward the oropharynx such that the additional portion of the nasal wash fluid is added to the mixture in the oropharynx.
Inventive Concept 183. The method according to Inventive Concept 182, wherein the method further includes, after sniffing the additional portion of the nasal wash fluid from the nasal cavity toward the oropharynx, again gargling the mixture.
Inventive Concept 184. The method according to Inventive Concept 182, wherein gargling the mixture and thereafter sniffing the additional portion of the nasal wash fluid includes repeating the steps of gargling and sniffing at least two times while at least a portion of the nasal wash fluid remains in the oropharynx.
Inventive Concept 185. The method according to any one of Inventive Concepts 169 and 170, wherein the method further includes, after gargling the mixture and before collecting the specimen sample, while holding the mixture in the oropharynx, snorting an additional portion of the nasal wash fluid from the nasal cavity toward the oropharynx such that the additional portion of the nasal wash fluid is added to the mixture in the oropharynx.
Inventive Concept 186. The method according to Inventive Concept 185, wherein the method further includes, after snorting the additional portion of the nasal wash fluid from the nasal cavity toward the oropharynx, again gargling the mixture.
Inventive Concept 187. The method according to Inventive Concept 185, wherein gargling the mixture and thereafter snorting the additional portion of the nasal wash fluid includes repeating the steps of gargling and snorting at least two times while at least a portion of the nasal wash fluid remains in the oropharynx.
Inventive Concept 188. The method according to any one of Inventive Concepts 169, 170, and 171, further including, before collecting the specimen sample, spraying, via the anterior opening of the oral cavity, the oropharynx with oropharyngeal fluid wash.
Inventive Concept 189. The method according to any one of Inventive Concepts 169 and 170, further including swishing, by the subject, the mixture in the oral cavity.
Inventive Concept 190. The method according to any one of Inventive Concepts 83-113, further including, before collecting the specimen sample, spraying, via the anterior opening of the oral cavity, the oropharynx with oropharyngeal fluid wash.
Inventive Concept 191. The method according to any one of Inventive Concepts 83-113, wherein the specimen sample is a first specimen sample that contains at least a first portion of the nasal wash fluid and at least a first portion of the biological material washed into the oropharynx, and

wherein the method further includes:
collecting from the nasal cavity, via one or two nostrils of the subject, a second specimen sample that contains at least a second portion of the nasal wash fluid and at least a second portion of the biological material in the nasal cavity; and
combining the first and the second specimen samples to produce a combined specimen sample.
   Inventive Concept 192. The method according to Inventive Concept 191, wherein collecting the second specimen sample includes aspirating the second specimen sample via the one or two nostrils.
   Inventive Concept 193. The method according to Inventive Concept 191, wherein collecting the second specimen sample includes blowing, by the subject, the second specimen sample out of the nasal cavity via the one or two nostrils.
   Inventive Concept 194. The method according to Inventive Concept 191, wherein collecting the second specimen sample includes collecting the second specimen sample that was blown, by the subject, out of the nasal cavity via the one or two nostrils.
   Inventive Concept 195. The method according to Inventive Concept 191, wherein collecting the second specimen sample includes swabbing the nasal cavity.
   Inventive Concept 196. The method according to any one of Inventive Concepts 83-113,

wherein intranasally dispensing the nasal wash fluid into the nasal cavity includes inserting a tubular applicator of a nasal wash fluid dispenser into a nostril of the subject and intranasally dispensing nasal wash fluid from the tubular applicator, and

wherein the nasal wash fluid dispenser is shaped so as to guide introduction of the tubular applicator into the nostril at a specific orientation with respect to a maxilla of the subject.

Inventive Concept 197. The method according to Inventive Concept 196, wherein the dispensing container is shaped so as to guide the introduction of the tubular applicator into the nostril at the specific orientation with respect to the maxilla.
Inventive Concept 198. The method according to Inventive Concept 196, wherein the nasal wash fluid dispenser includes a housing that is shaped so as to guide the introduction of the tubular applicator into the nostril at the specific orientation with respect to the maxilla.
Inventive Concept 199. The method according to Inventive Concept 196,

wherein the nasal wash fluid dispenser is shaped so as to define a concave surface that faces in a posterior direction and generally conforms to an outer surface of an upper lip of the subject, and

wherein inserting the tubular applicator into the nostril includes pressing the concave surface against the upper lip so as to stabilize the nasal wash fluid dispenser with respect to the maxilla.

Inventive Concept 200. The method according to Inventive Concept 199, wherein the concave surface has a lateral width of between 2.5 and 7 cm.
Inventive Concept 201. The method according to Inventive Concept 199, wherein the concave surface is curved.
Inventive Concept 202. The method according to Inventive Concept 199, wherein the concave surface is concave about an axis and a flat in a direction along the axis.
Inventive Concept 203. The method according to Inventive Concept 199, wherein the dispensing container is shaped so as to define the concave surface.
Inventive Concept 204. The method according to Inventive Concept 199, wherein the nasal wash fluid dispenser includes a housing that is shaped so as to define the concave surface.
Inventive Concept 205. The method according to any one of Inventive Concepts 83-113, wherein the method does not include swabbing the nasal cavity.
Inventive Concept 206. The method according to any one of Inventive Concepts 83-113,

wherein the method does not include swabbing the nasopharynx, and

wherein collecting the specimen sample includes swabbing the oropharynx.

Inventive Concept 207. The method according to Inventive Concept 206, wherein the method does not include swabbing any portion of the nasal cavity.
Inventive Concept 208. The method according to Inventive Concept 206, wherein collecting the specimen sample includes swabbing the oropharynx and swabbing one or both anterior nares of the nasal cavity.
Inventive Concept 209. The method according to Inventive Concept 206, wherein collecting the specimen sample includes swabbing the oropharynx and swabbing one or more turbinates of the nasal cavity selected from the group consisting of: a middle turbinate and an inferior turbinate.
Inventive Concept 210. The method according to any one of Inventive Concepts 83-113, wherein collecting the specimen sample that passed out of the anterior opening of the oral cavity includes collecting a specimen sample that passed out of the anterior opening of the oral cavity and was not swabbed from oropharynx.
Inventive Concept 211. The method according to any one of Inventive Concepts 83-113, wherein the method does not include swabbing the oropharynx and does not include swabbing the nasopharynx.
Inventive Concept 212. The method according to any one of Inventive Concepts 83-113, wherein collecting the specimen sample includes drawing the specimen sample out of the oral cavity via the anterior opening of the oral cavity using an absorbent material.
Inventive Concept 213. The method according to Inventive Concept 212, wherein the absorbent material is located on a tip of a collector shaft, and wherein drawing the specimen sample out of the oral cavity via the anterior opening of the oral cavity using the absorbent material includes inserting the tip of the collector shaft into the oral cavity.
Inventive Concept 214. The method according to Inventive Concept 213, wherein drawing the specimen sample out of the oral cavity via the anterior opening of the oral cavity using the absorbent material includes sucking, by the subject, on the absorbent material.
Inventive Concept 215. The method according to Inventive Concept 212, wherein drawing the specimen sample out of the oral cavity includes contacting one or more portions of the oral cavity with the absorbent material, without swabbing the oropharynx.
Inventive Concept 216. The method according to Inventive Concept 215, wherein the one or more portions of the oral cavity include one or more portions selected from the group consisting of: buccal mucosa, a tongue, gums, and palatal mucosa.
Inventive Concept 217. The method according to any one of Inventive Concepts 83-113, wherein the method does not include swabbing any portion of the subject.

There is still further provided, in accordance with an Inventive Concept 218 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx;

thereafter, collecting a specimen sample by swabbing the oropharynx via an anterior opening of an oral cavity of the subject, the specimen sample containing at least a portion of the biological material washed into the oropharynx by the nasal wash fluid; and

thereafter, deriving information from extracellular vesicles present in the specimen sample,

wherein the method does not include swabbing the nasopharynx.

There is additionally provided, in accordance with an Inventive Concept 219 of the present invention, a method including:

intranasally dispensing nasal wash fluid into a nasal cavity of a subject such that the nasal wash fluid passes from the nasal cavity into an oropharynx of the subject via a nasopharynx of the subject;

thereafter, collecting a specimen sample by swabbing the oropharynx via an anterior opening of an oral cavity of the subject, the specimen sample containing at least a portion of the nasal wash fluid; and

thereafter, deriving information from extracellular vesicles present in the specimen sample.

Inventive Concept 220. The method according to any one of Inventive Concepts 218 and 219, wherein deriving the information includes performing an assay to derive the information.
Inventive Concept 221. The method according to any one of Inventive Concepts 218 and 219, wherein deriving the information includes ascertaining whether the extracellular vesicles present in the specimen sample include a biological marker.
Inventive Concept 222. The method according to any one of Inventive Concepts 218 and 219, wherein the extracellular vesicles are exosomes, and wherein deriving the information includes deriving the information from the exosomes present in the specimen sample.
Inventive Concept 223. The method according to any one of Inventive Concepts 218 and 219, wherein the extracellular vesicles are microvesicles, and wherein deriving the information includes deriving the information from the microvesicles present in the specimen sample.
Inventive Concept 224. The method according to any one of Inventive Concepts 218 and 219, wherein the extracellular vesicles are apoptotic bodies, and wherein deriving the information includes deriving the information from the apoptotic bodies present in the specimen sample.
Inventive Concept 225. The method according to any one of Inventive Concepts 218 and 219, wherein deriving the information from the extracellular vesicles includes identifying one or more proteins of the extracellular vesicles.
Inventive Concept 226. The method according to any one of Inventive Concepts 218 and 219, wherein deriving the information from the extracellular vesicles includes identifying one or more nucleic acids of the extracellular vesicles.
Inventive Concept 227. The method according to any one of Inventive Concepts 218 and 219, wherein the method does not include swabbing the nasal cavity.
Inventive Concept 228. The method according to any one of Inventive Concepts 218 and 219, wherein collecting the specimen sample includes swabbing the oropharynx and swabbing one or both anterior nares of the nasal cavity.
Inventive Concept 229. The method according to any one of Inventive Concepts 218 and 219, wherein collecting the specimen sample includes swabbing the oropharynx and swabbing one or more turbinates of the nasal cavity selected from the group consisting of: a middle turbinate and an inferior turbinate.
Inventive Concept 230. The method according to any one of Inventive Concepts 218 and 219, wherein collecting the specimen sample includes collecting the specimen sample after sensing, by the subject, the nasal wash fluid in the oropharynx.
Inventive Concept 231. The method according to Inventive Concept 230, wherein collecting the specimen sample includes collecting the specimen sample after tasting, by the subject, the nasal wash fluid in the oropharynx.
Inventive Concept 232. The method according to Inventive Concept 231, wherein the nasal wash fluid includes a tastant, and wherein collecting the specimen sample includes collecting the specimen sample after tasting, by the subject, the tastant in the oropharynx.
Any of Inventive Concepts 218-232 may be performed in combination with any of Inventive Concepts 83-113, 128-167, and 190-205.

There is yet additionally provided, in accordance with an Inventive Concept 233 of the present invention, a method including:

intranasally dispensing nasal wash fluid into a nasal cavity of a subject such that the nasal wash fluid passes from the nasal cavity into an oropharynx of the subject via a nasopharynx of the subject;

thereafter, collecting a specimen sample that passed out of an anterior opening of an oral cavity of the subject and contains at least a portion of the nasal wash fluid; and

thereafter, deriving information from extracellular vesicles present in the specimen sample.

Inventive Concept 234. The method according to Inventive Concept 233, wherein deriving the information includes performing an assay to derive the information.
Inventive Concept 235. The method according to Inventive Concept 233, wherein deriving the information includes ascertaining whether the extracellular vesicles present in the specimen sample include a biological marker.
Inventive Concept 236. The method according to Inventive Concept 233, wherein the extracellular vesicles are exosomes, and wherein deriving the information includes deriving the information from the exosomes present in the specimen sample.
Inventive Concept 237. The method according to Inventive Concept 233, wherein the extracellular vesicles are microvesicles, and wherein deriving the information includes deriving the information from the microvesicles present in the specimen sample.
Inventive Concept 238. The method according to Inventive Concept 233, wherein the extracellular vesicles are apoptotic bodies, and wherein deriving the information includes deriving the information from the apoptotic bodies present in the specimen sample.
Inventive Concept 239. The method according to Inventive Concept 233, wherein deriving the information from the extracellular vesicles includes identifying one or more proteins of the extracellular vesicles.
Inventive Concept 240. The method according to Inventive Concept 233, wherein deriving the information from the extracellular vesicles includes identifying one or more nucleic acids of the extracellular vesicles.
Inventive Concept 241. The method according to Inventive Concept 233, wherein collecting the specimen sample includes collecting the specimen sample into a collection receptacle.
Inventive Concept 242. The method according to Inventive Concept 233, wherein intranasally dispensing includes intranasally dispensing, by the subject, the nasal wash fluid into the nasal cavity.
Inventive Concept 243. The method according to Inventive Concept 233, wherein collecting the specimen sample includes expressing, by the subject, the specimen sample out of the oropharynx via the anterior opening of the oral cavity.
Inventive Concept 244. The method according to Inventive Concept 243, wherein expressing the specimen sample includes spitting, by the subject, the specimen sample out of the oropharynx via the anterior opening of the oral cavity.
Inventive Concept 245. The method according to Inventive Concept 243, wherein expressing the specimen sample includes expressing the specimen sample out of the oropharynx via the anterior opening of the oral cavity after sensing, by the subject, the nasal wash fluid in the oropharynx.
Inventive Concept 246. The method according to Inventive Concept 245, wherein expressing the specimen sample includes expressing the specimen sample out of the oropharynx via the anterior opening of the oral cavity after tasting, by the subject, the nasal wash fluid in the oropharynx.
Inventive Concept 247. The method according to Inventive Concept 246, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 248. The method according to Inventive Concept 243, wherein intranasally dispensing includes intranasally dispensing, by a healthcare worker, the nasal wash fluid into the nasal cavity.
Inventive Concept 249. The method according to Inventive Concept 233, further including, before collecting the specimen sample, tilting back, by the subject, a head of the subject.
Inventive Concept 250. The method according to Inventive Concept 233, further including, while intranasally dispensing the nasal wash fluid, tilting back, by the subject, a head of the subject.
Inventive Concept 251. The method according to Inventive Concept 233, further including, before intranasally dispensing the nasal wash fluid, tilting back, by the subject, a head of the subject.
Inventive Concept 252. The method according to Inventive Concept 233, further including, after intranasally dispensing the nasal wash fluid, tilting a head of the subject farther back than while intranasally dispensing the nasal wash fluid and farther back than a neutral position.
Inventive Concept 253. The method according to Inventive Concept 233, wherein intranasally dispensing the nasal wash fluid includes intranasally dispensing the nasal wash fluid into the nasal cavity while a head of the subject is held in a neutral position or a tilted-forward position.
Inventive Concept 254. The method according to Inventive Concept 233, further including waiting for a period of time between (a) concluding intranasally dispensing the nasal wash fluid into the nasal cavity and (b) collecting the specimen sample, the period of time at least 5 minutes and less than 90 minutes.
Inventive Concept 255. The method according to Inventive Concept 233, wherein intranasally dispensing the nasal wash fluid includes intranasally dispensing the nasal wash fluid as one or more types of fluid discharge selected from the group consisting of: a fluid stream, a spray, a mist, a pressurized aerosol, and an atomized fluid.
Inventive Concept 256. The method according to Inventive Concept 233, wherein intranasally dispensing the nasal wash fluid includes intranasally dispensing the nasal wash fluid as steam.
Inventive Concept 257. The method according to Inventive Concept 233, wherein intranasally dispensing the nasal wash fluid includes intranasally dispensing the nasal wash fluid in a circular rotary motion to generate dispersion within the nasal cavity.
Inventive Concept 258. The method according to Inventive Concept 233, wherein intranasally dispensing the nasal wash fluid into the nasal cavity includes intranasally dispensing a total volume of 0.2-20 mL of the nasal wash fluid into the nasal cavity.
Inventive Concept 259. The method according to Inventive Concept 258, wherein intranasally dispensing the nasal wash fluid into the nasal cavity includes intranasally dispensing a total volume of 0.6-20 mL of the nasal wash fluid into the nasal cavity.
Inventive Concept 260. The method according to Inventive Concept 258, wherein intranasally dispensing the nasal wash fluid into the nasal cavity includes intranasally dispensing a total volume of 0.2-40 mL of the nasal wash fluid into the nasal cavity.
Inventive Concept 261. The method according to Inventive Concept 260, wherein intranasally dispensing the nasal wash fluid into the nasal cavity includes intranasally dispensing a total volume of 0.2-5 mL of the nasal wash fluid into the nasal cavity.
Inventive Concept 262. The method according to Inventive Concept 261, wherein intranasally dispensing the nasal wash fluid into the nasal cavity includes intranasally dispensing a total volume of 0.6-5 mL of the nasal wash fluid into the nasal cavity.
Inventive Concept 263. The method according to Inventive Concept 258, wherein intranasally dispensing the nasal wash fluid into the nasal cavity includes intranasally dispensing the total volume of the nasal wash fluid in a plurality of pulses.
Inventive Concept 264. The method according to Inventive Concept 263, wherein each of the pulses has a volume of 0.02-10 mL.
Inventive Concept 265. The method according to Inventive Concept 264, wherein each of the pulses has a volume of 0.02-1 mL.
Inventive Concept 266. The method according to Inventive Concept 233, wherein intranasally dispensing the nasal wash fluid into the nasal cavity includes intranasally dispensing the nasal wash fluid with a sprayed particle size of 5-50 microns.
Inventive Concept 267. The method according to Inventive Concept 266, wherein intranasally dispensing the nasal wash fluid includes intranasally dispensing the nasal wash fluid with a sprayed particle size of 5-15 microns.
Inventive Concept 268. The method according to Inventive Concept 266, wherein intranasally dispensing the nasal wash fluid includes intranasally dispensing the nasal wash fluid with a sprayed particle size of 15-20 microns.
Inventive Concept 269. The method according to Inventive Concept 233, wherein intranasally dispensing the nasal wash fluid into the nasal cavity includes intranasally dispensing the nasal wash fluid into the nasal cavity while the subject is lying supine.
Inventive Concept 270. The method according to Inventive Concept 233, wherein collecting the specimen sample includes drawing the specimen sample out of the oropharynx via the anterior opening of the oral cavity using an absorbent material, without swabbing the oral cavity or the oropharynx.
Inventive Concept 271. The method according to any one of Inventive Concepts 233-270, wherein intranasally dispensing includes intranasally dispensing, by a healthcare worker, the nasal wash fluid into the nasal cavity.
Inventive Concept 272. The method according to Inventive Concept 271, wherein collecting the specimen sample includes receiving the specimen sample by a healthcare worker from the subject.
Inventive Concept 273. The method according to Inventive Concept 272, wherein receiving the specimen sample includes receiving the specimen sample that was expressed out of the oropharynx via the anterior opening of the oral cavity.
Inventive Concept 274. The method according to Inventive Concept 273, wherein receiving the specimen sample includes receiving the specimen sample that was spit out of the oropharynx via the anterior opening of the oral cavity.
Inventive Concept 275. The method according to Inventive Concept 271, wherein collecting the specimen sample includes aspirating the specimen sample out of the oropharynx via the anterior opening of the oral cavity.
Inventive Concept 276. The method according to Inventive Concept 271, wherein collecting the specimen sample includes instructing the subject, by the healthcare worker, to express the specimen sample out of the oropharynx via the anterior opening of the oral cavity.
Inventive Concept 277. The method according to Inventive Concept 276, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 278. The method according to any one of Inventive Concepts 233-270, further including, after intranasally dispensing the nasal wash fluid and before collecting the specimen sample, sniffing, by the subject, the nasal wash fluid from the nasal cavity posteriorly toward the nasopharynx.
Inventive Concept 279. The method according to any one of Inventive Concepts 233-270, further including, after intranasally dispensing the nasal wash fluid and before collecting the specimen sample, snorting, by the subject, the nasal wash fluid from the nasal cavity toward the nasopharynx.
Inventive Concept 280. The method according to any one of Inventive Concepts 233-270, wherein intranasally dispensing the nasal wash fluid into the nasal cavity includes intranasally dispensing the nasal wash fluid unidirectionally within the nasal cavity.
Inventive Concept 281. The method according to Inventive Concept 280, wherein intranasally dispensing the nasal wash fluid unidirectionally within the nasal cavity includes intranasally dispensing the nasal wash fluid unidirectionally within the nasal cavity in a superior direction.
Inventive Concept 282. The method according to Inventive Concept 280, wherein intranasally dispensing the nasal wash fluid unidirectionally within the nasal cavity includes intranasally dispensing the nasal wash fluid unidirectionally within the nasal cavity in a superoposterior direction.
Inventive Concept 283. The method according to Inventive Concept 282, wherein intranasally dispensing the nasal wash fluid unidirectionally within the nasal cavity in a superoposterior direction includes intranasally dispensing the nasal wash fluid unidirectionally within the nasal cavity toward an olfactory area of the nasal cavity.
Inventive Concept 284. The method according to Inventive Concept 280, wherein intranasally dispensing the nasal wash fluid unidirectionally within the nasal cavity includes intranasally dispensing the nasal wash fluid unidirectionally within the nasal cavity in a posterior direction.
Inventive Concept 285. The method according to Inventive Concept 284, wherein intranasally dispensing the nasal wash fluid unidirectionally within the nasal cavity in a posterior direction includes intranasally dispensing the nasal wash fluid unidirectionally within the nasal cavity toward the nasopharynx.
Inventive Concept 286. The method according to any one of Inventive Concepts 233-270, wherein intranasally dispensing the nasal wash fluid into the nasal cavity includes intranasally dispensing the nasal wash fluid multidirectionally within the nasal cavity.
Inventive Concept 287. The method according to Inventive Concept 286, wherein intranasally dispensing the nasal wash fluid multidirectionally within the nasal cavity includes dispensing the nasal wash fluid into the nasal cavity:

in a posterior direction toward the nasopharynx, and

in a superoposterior direction toward an olfactory area of the nasal cavity.

Inventive Concept 288. The method according to Inventive Concept 287, wherein intranasally dispensing the nasal wash fluid multidirectionally within the nasal cavity includes dispensing the nasal wash fluid into the nasal cavity additionally in at least a superior direction toward an inferior border of upper lateral cartilage of the nasal cavity.
Inventive Concept 289. The method according to Inventive Concept 288, wherein intranasally dispensing the nasal wash fluid multidirectionally within the nasal cavity includes dispensing the nasal wash fluid into the nasal cavity additionally bilaterally toward a septum and a lateral wall of the nasal cavity.
Inventive Concept 290. The method according to Inventive Concept 286, wherein intranasally dispensing the nasal wash fluid multidirectionally within the nasal cavity includes intranasally dispensing the nasal wash fluid via a plurality of nozzles.
Inventive Concept 291. The method according to Inventive Concept 290, wherein intranasally dispensing the nasal wash fluid multidirectionally within the nasal cavity via the plurality of nozzles includes intranasally dispensing the nasal wash fluid into the nasal cavity:

in a posterior direction toward the nasopharynx via a first subset of one or more of the plurality of nozzles, and in a superoposterior direction toward an olfactory area of the nasal cavity via a second subset of one or more of the plurality of nozzles, the first and the second subsets not including any nozzles common to both subsets.

Inventive Concept 292. The method according to Inventive Concept 291,

wherein the plurality of nozzles includes only first and second nozzles, and wherein the first and the second subsets consist of the first and the second nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle, and
in the superoposterior direction toward the olfactory area of the nasal cavity via the second nozzle.
   Inventive Concept 293. The method according to Inventive Concept 291, wherein intranasally dispensing the nasal wash fluid multidirectionally within the nasal cavity via the plurality of nozzles includes intranasally dispensing the nasal wash fluid into the nasal cavity:

in the posterior direction toward the nasopharynx via the first subset of one or more of the plurality of nozzles,

in the superoposterior direction toward the olfactory area of the nasal cavity via the second subset of one or more of the plurality of nozzles, and

at least in a superior direction toward an inferior border of upper lateral cartilage of the nasal cavity via a third subset of one or more of the plurality of nozzles, wherein none of the nozzles is included in more than one of the first, the second, and the third subsets.

Inventive Concept 294. The method according to Inventive Concept 293, wherein intranasally dispensing the nasal wash fluid in at least the superior direction toward the inferior border of upper lateral cartilage includes dispensing the nasal wash fluid into the nasal cavity additionally bilaterally toward a septum and a lateral wall of the nasal cavity via the third subset of one or more of the plurality of nozzles.
Inventive Concept 295. The method according to Inventive Concept 293,

wherein the plurality of nozzles includes only first, second, and third nozzles, and wherein the first, the second, and the third subsets consist of the first, the second, and the third nozzles, respectively, and

wherein intranasally dispensing the nasal wash fluid into the nasal cavity includes intranasally dispensing the nasal wash fluid into the nasal cavity:
in the posterior direction toward the nasopharynx via the first nozzle,
in the superoposterior direction toward the olfactory area of the nasal cavity via the second nozzle, and
at least in the superior direction toward the inferior border of upper lateral cartilage of the nasal cavity via the third nozzle.
   Inventive Concept 296. The method according to Inventive Concept 290, wherein intranasally dispensing the nasal wash fluid multidirectionally within the nasal cavity includes intranasally dispensing the nasal wash fluid within the nasal cavity via the plurality of nozzles with respective different fluid flow characteristics, the fluid flow characteristics including one or more characteristics selected from the group consisting of: volume, sprayed particle size, velocity, and flow rate.
   Inventive Concept 297. The method according to any one of Inventive Concepts 233-270, wherein intranasally dispensing the nasal wash fluid into the nasal cavity includes intranasally dispensing the nasal wash fluid via both nostrils of the subject simultaneously.
   Inventive Concept 298. The method according to any one of Inventive Concepts 233-270, wherein the nasal wash fluid does not include a drug molecule.
   Inventive Concept 299. The method according to any one of Inventive Concepts 233-270, wherein the nasal wash fluid includes a non-irritant solution.
   Inventive Concept 300. The method according to Inventive Concept 299, wherein the non-irritant solution consists of water.
   Inventive Concept 301. The method according to Inventive Concept 299, wherein the non-irritant solution includes saline solution.
   Inventive Concept 302. The method according to Inventive Concept 301, wherein the non-irritant saline solution includes phosphate-buffered saline solution.
   Inventive Concept 303. The method according to any one of Inventive Concepts 233-270, wherein the nasal wash fluid includes a tastant.
   Inventive Concept 304. The method according to any one of Inventive Concepts 233-270, wherein the nasal wash fluid includes a color additive, and collecting the specimen sample includes confirming that the specimen sample contains at least a portion of the nasal wash fluid by visually ascertaining that the specimen sample includes the color additive.
   Inventive Concept 305. The method according to any one of Inventive Concepts 233-270, wherein the nasal wash fluid includes a chemical additive, and collecting the specimen sample includes confirming that the specimen sample contains at least a portion of the nasal wash fluid by ascertaining that the specimen sample includes the chemical additive.
   Inventive Concept 306. The method according to any one of Inventive Concepts 233-270, further including, after intranasally dispensing and before collecting the specimen sample, swishing in the oral cavity, by the subject, the nasal wash fluid washed into the oropharynx.
   Inventive Concept 307. The method according to any one of Inventive Concepts 233-270, further including, after intranasally dispensing and before collecting the specimen sample, gargling, by the subject, the nasal wash fluid washed into the oropharynx.
   Inventive Concept 308. The method according to Inventive Concept 307, wherein the method further includes, after gargling the nasal wash fluid and before collecting the specimen sample, while holding the nasal wash fluid in the oropharynx, sniffing an additional portion of the nasal wash fluid from the nasal cavity toward the oropharynx such that the additional portion of the nasal wash fluid is added to the nasal wash fluid already in the oropharynx.
   Inventive Concept 309. The method according to Inventive Concept 308, wherein the method further includes, after sniffing the additional portion of the nasal wash fluid from the nasal cavity toward the oropharynx, again gargling the nasal wash fluid washed into the oropharynx.
   Inventive Concept 310. The method according to any one of Inventive Concepts 233-270,

wherein the method further includes, before collecting the specimen sample:
orally dispensing oral wash fluid into the oral cavity via the anterior opening of the oral cavity; and
swishing, by the subject, the oral wash fluid in the oral cavity, and

wherein collecting includes collecting the specimen sample that passed out of the anterior opening of the oral cavity and contains at least a portion of the oral wash fluid and at least a portion of the nasal wash fluid.

Inventive Concept 311. The method according to any one of Inventive Concepts 233-270,

wherein the method further includes, before collecting the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein collecting includes collecting the specimen sample that passed out of the anterior opening of the oral cavity and contains at least a portion of the mixture.

Inventive Concept 312. The method according to any one of Inventive Concepts 233-270,

wherein the method further includes, before collecting the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein collecting includes collecting the specimen sample that passed out of the anterior opening of the oral cavity and contains at least a portion of the mixture.

Inventive Concept 313. The method according to any one of Inventive Concepts 233-270,

wherein the method further includes, before collecting the specimen sample:
orally dispensing oral wash fluid into the oral cavity via the anterior opening of the oral cavity; and
gargling, by the subject, the oral wash fluid, and

wherein collecting includes collecting the specimen sample that passed out of the anterior opening of the oral cavity and contains at least a portion of the oral wash fluid and at least a portion of the nasal wash fluid.

Inventive Concept 314. The method according to any one of Inventive Concepts 311, 312, and 313, wherein orally dispensing the oral wash fluid into the oral cavity includes orally dispensing the oral wash fluid into the oral cavity prior to intranasally dispensing the nasal wash fluid into the nasal cavity.
Inventive Concept 315. The method according to any one of Inventive Concepts 311, 312, and 313, wherein orally dispensing the oral wash fluid into the oral cavity includes orally dispensing the oral wash fluid into the oral cavity after intranasally dispensing the nasal wash fluid into the nasal cavity.
Inventive Concept 316. The method according to Inventive Concept 315, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 317. The method according to any one of Inventive Concepts 311, 312, and 313,

wherein the specimen sample is a first specimen sample, and the mixture is a first mixture,

wherein the method further includes, after collecting the first specimen sample:
waiting for a period of time, the period of time at least 5 minutes and less than 90 minutes;
thereafter, orally dispensing additional oral wash fluid into the oral cavity via the anterior opening of the oral cavity; and
thereafter, gargling, by the subject,

wherein collecting includes collecting a second specimen sample that passed out of the anterior opening of the oral cavity and contains at least a portion of the additional oral wash fluid, and

wherein the method does not include intranasally dispensing nasal wash fluid into the nasal cavity after collecting the first specimen sample.

Inventive Concept 318. The method according to any one of Inventive Concepts 311, 312, and 313,

wherein the nasal wash fluid includes a first color additive,

wherein the oral wash fluid includes a second colored additive, the first and the second color additives having different colors, and

wherein collecting the specimen sample includes confirming that the specimen sample contains at least a portion of the nasal wash fluid and at least a portion of the oral wash fluid by visually ascertaining that the specimen sample has a color produced by a combination of the first and the second color additives.

Inventive Concept 319. The method according to any one of Inventive Concepts 311, 312, and 313, wherein the oral wash fluid includes a non-irritant solution.
Inventive Concept 320. The method according to Inventive Concept 319, wherein the non-irritant solution consists of water.
Inventive Concept 321. The method according to Inventive Concept 319, wherein the non-irritant solution includes a tastant.
Inventive Concept 322. The method according to Inventive Concept 319, wherein the non-irritant solution includes saline solution.
Inventive Concept 323. The method according to Inventive Concept 322, wherein the non-irritant saline solution includes phosphate-buffered saline solution.
Inventive Concept 324. The method according to any one of Inventive Concepts 311 and 312, wherein the method further includes, after gargling the mixture and before collecting the specimen sample, while holding the mixture in the oropharynx, sniffing an additional portion of the nasal wash fluid from the nasal cavity toward the oropharynx such that the additional portion of the nasal wash fluid is added to the mixture in the oropharynx.
Inventive Concept 325. The method according to Inventive Concept 324, wherein the method further includes, after sniffing the additional portion of the nasal wash fluid from the nasal cavity toward the oropharynx, again gargling the mixture.
Inventive Concept 326. The method according to Inventive Concept 324, wherein gargling the mixture and thereafter sniffing the additional portion of the nasal wash fluid includes repeating the steps of gargling and sniffing at least two times while at least a portion of the nasal wash fluid remains in the oropharynx.
Inventive Concept 327. The method according to any one of Inventive Concepts 311 and 312, wherein the method further includes, after gargling the mixture and before collecting the specimen sample, while holding the mixture in the oropharynx, snorting an additional portion of the nasal wash fluid from the nasal cavity toward the oropharynx such that the additional portion of the nasal wash fluid is added to the mixture in the oropharynx.
Inventive Concept 328. The method according to Inventive Concept 327, wherein the method further includes, after snorting the additional portion of the nasal wash fluid from the nasal cavity toward the oropharynx, again gargling the mixture.
Inventive Concept 329. The method according to Inventive Concept 327, wherein gargling the mixture and thereafter snorting the additional portion of the nasal wash fluid includes repeating the steps of gargling and snorting at least two times while at least a portion of the nasal wash fluid remains in the oropharynx.
Inventive Concept 330. The method according to any one of Inventive Concepts 311 and 312, further including, before collecting the specimen sample, spraying, via the anterior opening of the oral cavity, the oropharynx with oropharyngeal fluid wash.
Inventive Concept 331. The method according to any one of Inventive Concepts 311 and 312, further including swishing, by the subject, the mixture in the oral cavity.
Inventive Concept 332. The method according to any one of Inventive Concepts 233-270, further including, before collecting the specimen sample, spraying, via the anterior opening of the oral cavity, the oropharynx with oropharyngeal fluid wash.
Inventive Concept 333. The method according to any one of Inventive Concepts 233-270,

wherein intranasally dispensing the nasal wash fluid into the nasal cavity includes inserting a tubular applicator of a nasal wash fluid dispenser into a nostril of the subject and intranasally dispensing the nasal wash fluid from the tubular applicator, and

wherein the nasal wash fluid dispenser is shaped so as to guide introduction of the tubular applicator into the nostril at a specific orientation with respect to a maxilla of the subject.

Inventive Concept 334. The method according to Inventive Concept 333, wherein the dispensing container is shaped so as to guide the introduction of the tubular applicator into the nostril at the specific orientation with respect to the maxilla.
Inventive Concept 335. The method according to Inventive Concept 333, wherein the nasal wash fluid dispenser includes a housing that is shaped so as to guide the introduction of the tubular applicator into the nostril at the specific orientation with respect to the maxilla.
Inventive Concept 336. The method according to Inventive Concept 333,

wherein the nasal wash fluid dispenser is shaped so as to define a concave surface that faces in a posterior direction and generally conforms to an outer surface of an upper lip of the subject, and

wherein inserting the tubular applicator into the nostril includes pressing the concave surface against the upper lip so as to stabilize the nasal wash fluid dispenser with respect to the maxilla.

Inventive Concept 337. The method according to Inventive Concept 336, wherein the concave surface has a lateral width of between 2.5 and 7 cm.
Inventive Concept 338. The method according to Inventive Concept 336, wherein the concave surface is curved.
Inventive Concept 339. The method according to Inventive Concept 336, wherein the concave surface is concave about an axis and flat in a direction along the axis.
Inventive Concept 340. The method according to Inventive Concept 336, wherein the dispensing container is shaped so as to define the concave surface.
Inventive Concept 341. The method according to Inventive Concept 336, wherein the nasal wash fluid dispenser includes a housing that is shaped so as to define the concave surface.
Inventive Concept 342. The method according to any one of Inventive Concepts 233-270, wherein the method does not include swabbing the nasal cavity.
Inventive Concept 343. The method according to any one of Inventive Concepts 233-270,

wherein the method does not include swabbing the nasopharynx, and

wherein collecting the specimen sample includes swabbing the oropharynx.

Inventive Concept 344. The method according to Inventive Concept 343, wherein the method does not include swabbing any portion of the nasal cavity.
Inventive Concept 345. The method according to Inventive Concept 343, wherein collecting the specimen sample includes swabbing the oropharynx and swabbing one or both anterior nares of the nasal cavity.
Inventive Concept 346. The method according to Inventive Concept 343, wherein collecting the specimen sample includes swabbing the oropharynx and swabbing one or more turbinates of the nasal cavity selected from the group consisting of: a middle turbinate and an inferior turbinate.
Inventive Concept 347. The method according to any one of Inventive Concepts 233-270, wherein the method does not include swabbing the oropharynx and does not include swabbing the nasopharynx.
Inventive Concept 348. The method according to any one of Inventive Concepts 233-270, wherein collecting the specimen sample includes drawing the specimen sample out of the oral cavity via the anterior opening of the oral cavity using an absorbent material.
Inventive Concept 349. The method according to Inventive Concept 348, wherein the absorbent material is located on a tip of a collector shaft, and wherein drawing the specimen sample out of the oral cavity via the anterior opening of the oral cavity using the absorbent material includes inserting the tip of the collector shaft into the oral cavity.
Inventive Concept 350. The method according to Inventive Concept 349, wherein drawing the specimen sample out of the oral cavity via the anterior opening of the oral cavity using the absorbent material includes sucking, by the subject, on the absorbent material.
Inventive Concept 351. The method according to Inventive Concept 348, wherein drawing the specimen sample out of the oral cavity includes contacting one or more portions of the oral cavity with the absorbent material, without swabbing the oropharynx.
Inventive Concept 352. The method according to Inventive Concept 351, wherein the one or more portions of the oral cavity include one or more portions selected from the group consisting of: buccal mucosa, a tongue, gums, and palatal mucosa.
Inventive Concept 353. The method according to any one of Inventive Concepts 233-270, wherein the method does not include swabbing any portion of the subject.

There is also provided, in accordance with an Inventive Concept 354 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx;

thereafter, expressing, by the subject, out of an anterior opening of an oral cavity of the subject, a specimen sample that contains at least a portion of the biological material washed into the oropharynx by the nasal wash fluid; and

thereafter, deriving information from extracellular vesicles present in the specimen sample.

Inventive Concept 355. The method according to Inventive Concept 354, wherein deriving the information includes performing an assay to derive the information.
Inventive Concept 356. The method according to Inventive Concept 354, wherein deriving the information includes ascertaining whether the extracellular vesicles present in the specimen sample include a biological marker.
Inventive Concept 357. The method according to Inventive Concept 354, wherein the extracellular vesicles are exosomes, and wherein deriving the information includes deriving the information from the exosomes present in the specimen sample.
Inventive Concept 358. The method according to Inventive Concept 354, wherein the extracellular vesicles are microvesicles, and wherein deriving the information includes deriving the information from the microvesicles present in the specimen sample.
Inventive Concept 359. The method according to Inventive Concept 354, wherein the extracellular vesicles are apoptotic bodies, and wherein deriving the information includes deriving the information from the apoptotic bodies present in the specimen sample.
Inventive Concept 360. The method according to Inventive Concept 354, wherein deriving the information from the extracellular vesicles includes identifying one or more proteins of the extracellular vesicles.
Inventive Concept 361. The method according to Inventive Concept 354, wherein deriving the information from the extracellular vesicles includes identifying one or more nucleic acids of the extracellular vesicles.
Inventive Concept 362. The method according to Inventive Concept 354, wherein expressing the specimen sample includes expressing the specimen sample, by the subject, into a collection receptacle.
Inventive Concept 363. The method according to Inventive Concept 354, wherein expressing the specimen sample includes spitting, by the subject, the specimen sample out of the oropharynx via the anterior opening of the oral cavity.
Inventive Concept 364. The method according to Inventive Concept 354, wherein expressing the specimen sample includes expressing the specimen sample out of the oropharynx via the anterior opening of the oral cavity after sensing, by the subject, the nasal wash fluid in the oropharynx.
Inventive Concept 365. The method according to Inventive Concept 364, wherein expressing the specimen sample includes expressing the specimen sample out of the oropharynx via the anterior opening of the oral cavity after tasting, by the subject, the nasal wash fluid in the oropharynx.
Inventive Concept 366. The method according to Inventive Concept 365, wherein the nasal wash fluid includes a tastant, and wherein expressing the specimen sample includes expressing the specimen sample out of the oropharynx via the anterior opening of the oral cavity after tasting, by the subject, the tastant in the oropharynx.
Inventive Concept 367. The method according to Inventive Concept 354, wherein intranasally dispensing includes intranasally dispensing, by the subject, the nasal wash fluid into the nasal cavity.
Inventive Concept 368. The method according to Inventive Concept 367, further including, before expressing the specimen sample, tilting back, by the subject, a head of the subject.
Inventive Concept 369. The method according to Inventive Concept 354, further including, while intranasally dispensing the nasal wash fluid, tilting back, by the subject, a head of the subject.
Inventive Concept 370. The method according to Inventive Concept 354, further including, before intranasally dispensing the nasal wash fluid, tilting back, by the subject, a head of the subject.
Inventive Concept 371. The method according to Inventive Concept 354, further including, after intranasally dispensing the nasal wash fluid, tilting a head of the subject farther back than while intranasally dispensing the nasal wash fluid and farther back than a neutral position.
Inventive Concept 372. The method according to Inventive Concept 354, wherein intranasally dispensing the nasal wash fluid includes intranasally dispensing the nasal wash fluid into the nasal cavity while a head of the subject is held in a neutral position or a tilted-forward position.
Inventive Concept 373. The method according to Inventive Concept 354, wherein intranasally dispensing includes intranasally dispensing, by a healthcare worker, the nasal wash fluid into the nasal cavity.
Inventive Concept 374. The method according to Inventive Concept 354, further including waiting for a period of time between (a) concluding intranasally dispensing the nasal wash fluid into the nasal cavity and (b) expressing the specimen sample, the period of time at least 10 minutes.
Inventive Concept 375. The method according to Inventive Concept 354, wherein intranasally dispensing the nasal wash fluid into the nasal cavity includes intranasally dispensing the nasal wash fluid into the nasal cavity while the subject is lying supine.
Inventive Concept 376. The method according to any one of Inventive Concepts 354-375, further including, after intranasally dispensing the nasal wash fluid and before expressing the specimen sample, sniffing, by the subject, the biological material with the nasal wash fluid from the nasal cavity posteriorly toward the nasopharynx.
Inventive Concept 377. The method according to any one of Inventive Concepts 354-375, further including, after intranasally dispensing the nasal wash fluid and before expressing the specimen sample, snorting, by the subject, the biological material with the nasal wash fluid from the nasal cavity toward the nasopharynx.
Inventive Concept 378. The method according to any one of Inventive Concepts 354-375,

wherein the method further includes, before expressing the specimen sample:
orally dispensing oral wash fluid into the oral cavity via the anterior opening of the oral cavity; and
swishing, by the subject, the oral wash fluid in the oral cavity, and

wherein expressing includes expressing, by the subject, out of the anterior opening of the oral cavity, the specimen sample that contains at least a portion of the oral wash fluid and the at least a portion of the biological material washed into the oropharynx with the nasal wash fluid.

Inventive Concept 379. The method according to any one of Inventive Concepts 354-375,

wherein the method further includes, before expressing the specimen sample:
orally dispensing oral wash fluid into the oral cavity via the anterior opening of the oral cavity such that the oral wash fluid forms a mixture with the nasal wash fluid in the oropharynx; and
gargling, by the subject, the mixture, and

wherein expressing includes expressing, by the subject, out of the anterior opening of the oral cavity, the specimen sample that contains at least a portion of the mixture and the at least a portion of the biological material washed into the oropharynx with the nasal wash fluid.

Inventive Concept 380. The method according to any one of Inventive Concepts 354-375,

wherein the method further includes, before expressing the specimen sample:
orally dispensing oral wash fluid into the oral cavity via the anterior opening of the oral cavity; and
gargling, by the subject, the oral wash fluid, such that the oral wash fluid forms a mixture with the nasal wash fluid, and

wherein expressing includes expressing, by the subject, out of the anterior opening of the oral cavity, the specimen sample that contains at least a portion of the mixture and the at least a portion of the biological material washed into the oropharynx with the nasal wash fluid.

Inventive Concept 381. The method according to any one of Inventive Concepts 354-375,

wherein the method further includes, before expressing the specimen sample:
orally dispensing oral wash fluid into the oral cavity via the anterior opening of the oral cavity; and
gargling, by the subject, the oral wash fluid, and

wherein expressing includes expressing, by the subject, out of the anterior opening of the oral cavity, the specimen sample that contains at least a portion of the oral wash fluid and the at least a portion of the biological material washed into the oropharynx with the nasal wash fluid.

Inventive Concept 382. The method according to any one of Inventive Concepts 379, 380, and 381, wherein orally dispensing the oral wash fluid into the oral cavity includes orally dispensing the oral wash fluid into the oral cavity after intranasally dispensing the nasal wash fluid into the nasal cavity.
Inventive Concept 383. The method according to Inventive Concept 382, further including waiting for a period of time between (a) concluding intranasally dispensing the nasal wash fluid into the nasal cavity and (b) orally dispensing the oral wash fluid into the oral cavity, the period of time at least 5 minutes and less than 90 minutes.
Inventive Concept 384. The method according to any one of Inventive Concepts 379, 380, and 381, further including swishing, by the subject, the mixture in the oral cavity.
Inventive Concept 385. The method according to any one of Inventive Concepts 354-375, wherein the method does not include swabbing the oropharynx and does not include swabbing the nasopharynx.
Inventive Concept 386. The method according to any one of Inventive Concepts 354-375, wherein the method does not include swabbing any portion of the subject.
Any of Inventive Concepts 354-386 may be performed in combination with any of Inventive Concepts 84-217.

There is further provided, in accordance with an Inventive Concept 387 of the present invention, a method including:

intranasally dispensing a vapor into a nasal cavity of a subject to loosen biological material in the nasal cavity;

thereafter, collecting a specimen sample that passed out of an anterior opening of an oral cavity of the subject and contains at least a portion of the biological material; and

thereafter, deriving information from extracellular vesicles present in the specimen sample.

Inventive Concept 388. The method according to Inventive Concept 387, wherein deriving the information includes performing an assay to derive the information.
Inventive Concept 389. The method according to Inventive Concept 387, wherein deriving the information includes ascertaining whether the extracellular vesicles present in the specimen sample include a biological marker.
Inventive Concept 390. The method according to Inventive Concept 387, wherein the extracellular vesicles are exosomes, and wherein deriving the information includes deriving the information from the exosomes present in the specimen sample.
Inventive Concept 391. The method according to Inventive Concept 387, wherein the extracellular vesicles are microvesicles, and wherein deriving the information includes deriving the information from the microvesicles present in the specimen sample.
Inventive Concept 392. The method according to Inventive Concept 387, wherein the extracellular vesicles are apoptotic bodies, and wherein deriving the information includes deriving the information from the apoptotic bodies present in the specimen sample.
Inventive Concept 393. The method according to Inventive Concept 387, wherein deriving the information from the extracellular vesicles includes identifying one or more proteins of the extracellular vesicles.
Inventive Concept 394. The method according to Inventive Concept 387, wherein deriving the information from the extracellular vesicles includes identifying one or more nucleic acids of the extracellular vesicles.
Inventive Concept 395. The method according to Inventive Concept 387, wherein intranasally dispensing the vapor includes inhaling, by the subject, the vapor into the nasal cavity.
Inventive Concept 396. The method according to Inventive Concept 395,

wherein inhaling the vapor into the nasal cavity includes inhaling the vapor into the nasal cavity and into an oropharynx of the subject via a nasopharynx of the subject, such that the vapor washes the biological material into the oropharynx from (a) the nasal cavity, (b) the nasopharynx, or (c) the nasal cavity and the nasopharynx, and

wherein collecting the specimen sample includes collecting the specimen sample that contains at least a portion of the biological material washed into the oropharynx by the vapor.

There is still further provided, in accordance with an Inventive Concept 397 of the present invention, a method including:

spraying, via an anterior opening of an oral cavity of a subject, an oropharynx of a subject with oropharyngeal fluid wash;

thereafter, collecting a specimen sample that passed out of the anterior opening of the oral cavity and contains at least a portion of biological material washed off a pharyngeal wall of the subject into an oropharynx of the subject by the oropharyngeal fluid wash; and

thereafter, deriving information from extracellular vesicles present in the specimen sample.

Inventive Concept 398. The method according to Inventive Concept 397, wherein deriving the information includes performing an assay to derive the information.
Inventive Concept 399. The method according to Inventive Concept 397, wherein deriving the information includes ascertaining whether the extracellular vesicles present in the specimen sample include a biological marker.
Inventive Concept 400. The method according to Inventive Concept 397, wherein the extracellular vesicles are exosomes, and wherein deriving the information includes deriving the information from the exosomes present in the specimen sample.
Inventive Concept 401. The method according to Inventive Concept 397, wherein the extracellular vesicles are microvesicles, and wherein deriving the information includes deriving the information from the microvesicles present in the specimen sample.
Inventive Concept 402. The method according to Inventive Concept 397, wherein the extracellular vesicles are apoptotic bodies, and wherein deriving the information includes deriving the information from the apoptotic bodies present in the specimen sample.
Inventive Concept 403. The method according to Inventive Concept 397, wherein deriving the information from the extracellular vesicles includes identifying one or more proteins of the extracellular vesicles.
Inventive Concept 404. The method according to Inventive Concept 397, wherein deriving the information from the extracellular vesicles includes identifying one or more nucleic acids of the extracellular vesicles.
Inventive Concept 405. The method according to Inventive Concept 397, wherein the method further includes, before collecting the specimen sample, gargling the oropharyngeal fluid wash by the subject.
Inventive Concept 406. The method according to Inventive Concept 397, wherein the method further includes, before collecting the specimen sample, swishing, by the subject, the oropharyngeal fluid in the oral cavity.
Inventive Concept 407. The method according to Inventive Concept 397, wherein the method does not include gargling the oropharyngeal fluid wash by the subject.
Inventive Concept 408. The method according to Inventive Concept 397,

wherein the method further includes, before collecting the specimen sample:
orally dispensing oral wash fluid into the oral cavity via the anterior opening of the oral cavity such that the oral wash fluid forms a mixture with the oropharyngeal fluid wash in the oropharynx; and
gargling, by the subject, the mixture, and

wherein collecting includes collecting the specimen sample that passed out of the anterior opening of the oral cavity and contains at least a portion of the mixture and the at least a portion of the biological material washed off the pharyngeal wall into the oropharynx by the mixture.

Inventive Concept 409. The method according to Inventive Concept 397, wherein spraying includes spraying while the subject says “ah” or “eh.”

410. The method according to Inventive Concept 397, wherein spraying includes spraying, by the subject, the oropharynx with the oropharyngeal fluid wash.

Inventive Concept 411. The method according to Inventive Concept 397, wherein spraying includes spraying a total volume of 2-15 mL of the oropharyngeal fluid wash.
Inventive Concept 412. The method according to Inventive Concept 397, wherein spraying includes spraying the oropharynx with the oropharyngeal fluid wash in a plurality of pulses.
Inventive Concept 413. The method according to Inventive Concept 412, wherein each of the pulses has a volume of 0.05-5 mL.
Inventive Concept 414. The method according to any one of Inventive Concepts 397-413, wherein collecting the specimen sample includes expressing, by the subject, the specimen sample out of the oropharynx via the anterior opening of the oral cavity.
Inventive Concept 415. The method according to Inventive Concept 414, wherein expressing the specimen sample includes spitting, by the subject, the specimen sample out of the oropharynx via the anterior opening of the oral cavity.
Inventive Concept 416. The method according to Inventive Concept 414, wherein spraying includes spraying, by a healthcare worker, the oropharynx with the oropharyngeal fluid wash.
Inventive Concept 417. The method according to any one of Inventive Concepts 397-413, wherein collecting the specimen sample includes drawing the specimen sample out of the oropharynx via the anterior opening of the oral cavity using an absorbent material, without swabbing the oral cavity or the oropharynx.
Inventive Concept 418. The method according to any one of Inventive Concepts 397-413, wherein spraying includes spraying, by a healthcare worker, the oropharynx with the oropharyngeal fluid wash.
Inventive Concept 419. The method according to Inventive Concept 418, wherein collecting the specimen sample includes receiving the specimen sample by a healthcare worker from the subject.
Inventive Concept 420. The method according to Inventive Concept 419, wherein receiving the specimen sample includes receiving the specimen sample that was expressed out of the oropharynx via the anterior opening of the oral cavity.
Inventive Concept 421. The method according to Inventive Concept 420, wherein receiving the specimen sample includes receiving the specimen sample that was spit out of the oropharynx via the anterior opening of the oral cavity.
Inventive Concept 422. The method according to Inventive Concept 418, wherein collecting the specimen sample includes aspirating the specimen sample out of the oropharynx via the anterior opening of the oral cavity.
Inventive Concept 423. The method according to Inventive Concept 418, wherein collecting the specimen sample includes instructing the subject, by the healthcare worker, to express the specimen sample out of the oropharynx via the anterior opening of the oral cavity.
Inventive Concept 424. The method according to Inventive Concept 423, wherein instructing the subject, by the healthcare worker, to express the specimen sample out of the oropharynx via the anterior opening of the oral cavity includes instructing the subject, by the healthcare worker, to spit the specimen sample out of the oropharynx via the anterior opening of the oral cavity.
Inventive Concept 425. The method according to any one of Inventive Concepts 397-413, wherein deriving the information from the extracellular vesicles present in the specimen sample includes concentrating the specimen sample by filtration prior to deriving the information, by passing the specimen sample through one or more filters.
Inventive Concept 426. The method according to any one of Inventive Concepts 397-413, wherein the oropharyngeal fluid wash includes a non-irritant solution.
Inventive Concept 427. The method according to Inventive Concept 426, wherein the non-irritant solution consists of water.
Inventive Concept 428. The method according to Inventive Concept 426, wherein the non-irritant solution includes saline solution.
Inventive Concept 429. The method according to Inventive Concept 428, wherein the non-irritant saline solution includes phosphate-buffered saline solution.
Inventive Concept 430. The method according to any one of Inventive Concepts 397-413, wherein the method does not include swabbing the oropharynx and does not include swabbing a nasopharynx of the subject.
Inventive Concept 431. The method according to any one of Inventive Concepts 397-413, wherein the method does not include swabbing any portion of the subject.

There is additionally provided, in accordance with an Inventive Concept 432 of the present invention, a method including:

spraying, via an anterior opening of an oral cavity of a subject, an oropharynx of a subject with oropharyngeal fluid wash;

thereafter, expressing, by the subject, out of the anterior opening of the oral cavity, a specimen sample that contains at least a portion of biological material washed off a pharyngeal wall of the subject into an oropharynx of the subject by the oropharyngeal fluid wash; and

thereafter, deriving information from extracellular vesicles present in the specimen sample.

Inventive Concept 433. The method according to Inventive Concept 432, wherein deriving the information includes performing an assay to derive the information.
Inventive Concept 434. The method according to Inventive Concept 432, wherein deriving the information includes ascertaining whether the extracellular vesicles present in the specimen sample include a biological marker.
Inventive Concept 435. The method according to Inventive Concept 432, wherein the extracellular vesicles are exosomes, and wherein deriving the information includes deriving the information from the exosomes present in the specimen sample.
Inventive Concept 436. The method according to Inventive Concept 432, wherein the extracellular vesicles are microvesicles, and wherein deriving the information includes deriving the information from the microvesicles present in the specimen sample.
Inventive Concept 437. The method according to Inventive Concept 432, wherein the extracellular vesicles are apoptotic bodies, and wherein deriving the information includes deriving the information from the apoptotic bodies present in the specimen sample.
Inventive Concept 438. The method according to Inventive Concept 432, wherein deriving the information from the extracellular vesicles includes identifying one or more proteins of the extracellular vesicles.
Inventive Concept 439. The method according to Inventive Concept 432, wherein deriving the information from the extracellular vesicles includes identifying one or more nucleic acids of the extracellular vesicles.
Inventive Concept 440. The method according to Inventive Concept 432, wherein the method further includes, before expressing the specimen sample, gargling the oropharyngeal fluid wash by the subject.
Inventive Concept 441. The method according to Inventive Concept 432, wherein the method further includes, before expressing the specimen sample, swishing, by the subject, the oropharyngeal fluid wash in the oral cavity.
Inventive Concept 442. The method according to Inventive Concept 397, wherein the method does not include gargling the oropharyngeal fluid wash by the subject.
Inventive Concept 443. The method according to Inventive Concept 397,

wherein the method further includes, before expressing the specimen sample:
orally dispensing oral wash fluid into the oral cavity via the anterior opening of the oral cavity such that the oral wash fluid forms a mixture with the oropharyngeal fluid wash in the oropharynx; and
gargling, by the subject, the mixture, and

wherein expressing includes expressing, through the anterior opening the oral cavity, the specimen sample that contains at least a portion of the mixture and the at least a portion of the biological material washed off the pharyngeal wall into the oropharynx by the mixture.

Inventive Concept 444. The method according to Inventive Concept 397, wherein spraying includes spraying while the subject says “ah” or “eh.”
Any of Inventive Concepts 432-444 may be performed in combination with any of Inventive Concepts 397-431.

There is yet additionally provided, in accordance with an Inventive Concept 445 of the present invention, a method including:

collecting, from a subject, a liquid specimen sample that includes saliva and an orally-dispensed oral wash fluid;

passing the liquid specimen sample through a filter to trap at least some of the saliva, the filter having a nominal pore size of between 30 microns and 1.5 mm, the nominal pore size representative of a minimum size of spherical particles necessary for the filter to retain 85% of the spherical particles when H₂O containing the spherical particles is passed through the filter at 20 degrees C. under pressure supplied by a 10 cm water column; and

deriving information from extracellular vesicles present in the saliva trapped by the filter.

Inventive Concept 446. The method according to Inventive Concept 445, wherein deriving the information includes performing an assay to derive the information.
Inventive Concept 447. The method according to Inventive Concept 445, wherein deriving the information includes ascertaining whether the extracellular vesicles present in the specimen sample include a biological marker.
Inventive Concept 448. The method according to Inventive Concept 445, wherein the extracellular vesicles are exosomes, and wherein deriving the information includes deriving the information from the exosomes present in the saliva trapped by the filter.
Inventive Concept 449. The method according to Inventive Concept 445, wherein the extracellular vesicles are microvesicles, and wherein deriving the information includes deriving the information from the microvesicles present in the saliva trapped by the filter.
Inventive Concept 450. The method according to Inventive Concept 445, wherein the extracellular vesicles are apoptotic bodies, and wherein deriving the information includes deriving the information from the apoptotic bodies present in the saliva trapped by the filter.
Inventive Concept 451. The method according to Inventive Concept 445, wherein deriving the information from the extracellular vesicles includes identifying one or more proteins of the extracellular vesicles.
Inventive Concept 452. The method according to Inventive Concept 445, wherein deriving the information from the extracellular vesicles includes identifying one or more nucleic acids of the extracellular vesicles.
Inventive Concept 453. The method according to Inventive Concept 445, wherein the nominal pore size is at least 40 microns.
Inventive Concept 454. The method according to Inventive Concept 453, wherein the nominal pore size is at least 50 microns.
Inventive Concept 455. The method according to Inventive Concept 454, wherein the nominal pore size is at least 60 microns.
Inventive Concept 456. The method according to Inventive Concept 455, wherein the nominal pore size is at least 100 microns.
Inventive Concept 457. The method according to Inventive Concept 456, wherein the nominal pore size is at least 120 microns.
Inventive Concept 458. The method according to Inventive Concept 457, wherein the nominal pore size is at least 150 microns.
Inventive Concept 459. The method according to Inventive Concept 458, wherein the nominal pore size is at least 200 microns.
Inventive Concept 460. The method according to Inventive Concept 459, wherein the nominal pore size is at least 500 microns.
Inventive Concept 461. The method according to Inventive Concept 445, wherein the nominal pore size is less than 1 mm.
Inventive Concept 462. The method according to Inventive Concept 461, wherein the nominal pore size is less than 750 microns.
Inventive Concept 463. The method according to Inventive Concept 462, wherein the nominal pore size less than 500 microns.
Inventive Concept 464. The method according to Inventive Concept 463, wherein the nominal pore size less than 250 microns.
Inventive Concept 465. The method according to Inventive Concept 464, wherein the nominal pore size less than 200 microns.
Inventive Concept 466. The method according to Inventive Concept 445, wherein the nominal pore size is between 50 and 200 microns.
Inventive Concept 467. The method according to Inventive Concept 466, wherein the nominal pore size is between 50 and 150 microns.
Inventive Concept 468. The method according to Inventive Concept 467, wherein the nominal pore size is between 100 and 150 microns.
Inventive Concept 469. The method according to Inventive Concept 445, wherein the method does not include centrifuging the liquid specimen sample.
Inventive Concept 470. The method according to Inventive Concept 445,

wherein the filter is one of a plurality of filters arranged in a series of filters, the filters having respective nominal pore sizes, all of which are between 30 microns and 1.5 mm,

wherein passing the liquid specimen sample through the filter includes passing the liquid specimen sample through the series of filters, without passing the liquid specimen sample through any other filters, and

wherein deriving the information from the extracellular vesicles present in the saliva includes deriving the information from the extracellular vesicles present in the saliva trapped by at least one of the plurality of filters.

Inventive Concept 471. The method according to Inventive Concept 470, wherein deriving the information from the extracellular vesicles present in the saliva includes deriving the information from the extracellular vesicles present in the saliva trapped by at least two of the plurality of filters.
Inventive Concept 472. The method according to Inventive Concept 445,

wherein the filter is a first filter, wherein passing the liquid specimen sample through the filter includes passing the liquid specimen sample through the first filter to produce a filtrate, and wherein deriving the information from the extracellular vesicles present in the saliva trapped by the filter includes deriving the information from the extracellular vesicles present in the saliva trapped by the first filter, and

wherein the method further includes:
passing the filtrate through a second filter to trap at least some of the saliva, and
deriving the information from the extracellular vesicles present in the saliva trapped by the second filter.
   Inventive Concept 473. The method according to Inventive Concept 472, wherein a nominal pore size of the second filter equals the nominal pore size of the first filter.
   Inventive Concept 474. The method according to Inventive Concept 472, wherein a nominal pore size of the second filter is between 0.03 and 21 microns.
   Inventive Concept 475. The method according to Inventive Concept 474, wherein the nominal pore size of the second filter is between 0.8 and 1.5 microns.
   Inventive Concept 476. The method according to Inventive Concept 445, wherein passing the liquid specimen sample through the filter includes passing the liquid specimen sample through the filter to trap at least some of the saliva by size-based filtration.
   Inventive Concept 477. The method according to Inventive Concept 445, wherein passing the liquid specimen sample through the filter includes passing the liquid specimen sample through the filter to trap at least some of the saliva by adhesion of the saliva to the filter.
   Inventive Concept 478. The method according to Inventive Concept 445, wherein the filter is a depth filter.
   Inventive Concept 479. The method according to Inventive Concept 445, wherein the filter includes fibers.
   Inventive Concept 480. The method according to Inventive Concept 479, wherein the filter includes crisscrossing fibers.
   Inventive Concept 481. The method according to Inventive Concept 445, wherein the filter includes a mesh filter.
   Inventive Concept 482. The method according to Inventive Concept 481, wherein the mesh filter includes woven mesh filter.
   Inventive Concept 483. The method according to Inventive Concept 481, wherein the mesh filter includes a non-woven mesh filter.
   Inventive Concept 484. The method according to Inventive Concept 445, wherein the filter includes a screen.
   Inventive Concept 485. The method according to Inventive Concept 484, wherein the screen includes a woven screen.
   Inventive Concept 486. The method according to Inventive Concept 484, wherein the screen includes a non-woven screen.
   Inventive Concept 487. The method according to Inventive Concept 445, wherein the filter includes a polymer.
   Inventive Concept 488. The method according to Inventive Concept 487, wherein the polymer includes a polyester.
   Inventive Concept 489. The method according to Inventive Concept 445, wherein passing the liquid specimen sample through the filter includes passing the liquid specimen sample through exactly one filter.
   Inventive Concept 490. The method according to Inventive Concept 445, wherein the method does not include swabbing a nasal cavity of the subject.
   Inventive Concept 491. The method according to Inventive Concept 445, wherein the method does not include swabbing a pharynx of the subject.
   Inventive Concept 492. The method according to Inventive Concept 445, wherein the method does not include swabbing a nasal cavity of the subject and does not include swabbing a pharynx of the subject.
   Inventive Concept 493. The method according to Inventive Concept 445, wherein the method does not include swabbing any portion of the subject.
   Inventive Concept 494. The method according to Inventive Concept 445, wherein collecting the liquid specimen sample includes collecting the liquid specimen sample that was expressed via an anterior opening of an oral cavity of the subject.
   Inventive Concept 495. The method according to Inventive Concept 445, wherein collecting the liquid specimen sample includes aspirating the liquid specimen sample via an anterior opening of an oral cavity of the subject.
   Inventive Concept 496. The method according to Inventive Concept 445, wherein collecting the liquid specimen sample includes:

gargling, by the subject, the oral wash fluid to produce the liquid specimen sample that includes the oral wash fluid and the saliva; and

thereafter, collecting the liquid specimen sample from the subject.

Inventive Concept 497. The method according to Inventive Concept 445, wherein collecting the liquid specimen sample includes:

gargling, by the subject, the oral wash fluid to produce the liquid specimen sample that includes the oral wash fluid and the saliva; and

thereafter, expressing, by the subject, out of an anterior opening of an oral cavity of the subject, the liquid specimen sample.

Inventive Concept 498. The method according to Inventive Concept 445, wherein collecting the liquid specimen sample includes:

swishing, by the subject, the oral wash fluid in an oral cavity of the subject to produce the liquid specimen sample that includes the oral wash fluid and the saliva; and

thereafter, collecting the liquid specimen sample from the subject.

Inventive Concept 499. The method according to Inventive Concept 445, wherein collecting the liquid specimen sample includes:

swishing, by the subject, the oral wash fluid in an oral cavity of the subject to produce the liquid specimen sample that includes the oral wash fluid and the saliva; and

thereafter, expressing, by the subject, out of an anterior opening of the oral cavity, the liquid specimen sample.

Inventive Concept 500. The method according to Inventive Concept 445, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 501. The method according to Inventive Concept 445, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, collecting the liquid specimen sample that passed out of an anterior opening of an oral cavity of the subject and includes the oral wash fluid, the nasal wash fluid, and at least a portion of the biological material washed into the oropharynx by the nasal wash fluid.

Inventive Concept 502. The method according to Inventive Concept 501, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

collecting the liquid specimen sample that passed out of the anterior opening of the oral cavity.

Inventive Concept 503. The method according to Inventive Concept 501, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

collecting the liquid specimen sample that passed out of the anterior opening of the oral cavity.

Inventive Concept 504. The method according to Inventive Concept 445, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, expressing, by the subject, out of an anterior opening of an oral cavity of the subject, the liquid specimen sample that includes the oral wash fluid, the nasal wash fluid, and at least a portion of the biological material washed into the oropharynx by the nasal wash fluid.

Inventive Concept 505. The method according to Inventive Concept 504, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

expressing, by the subject, the liquid specimen sample out of the anterior opening of the oral cavity.

Inventive Concept 506. The method according to Inventive Concept 504, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

expressing, by the subject, the liquid specimen sample out of the anterior opening of the oral cavity.

Inventive Concept 507. The method according to any one of Inventive Concepts 501 and 504, wherein intranasally dispensing includes intranasally dispensing, by the subject, the nasal wash fluid into the nasal cavity.
Inventive Concept 508. The method according to any one of Inventive Concepts 501 and 504, wherein intranasally dispensing includes intranasally dispensing, by a healthcare worker, the nasal wash fluid into the nasal cavity.

There is also provided, in accordance with an Inventive Concept 509 of the present invention, a method including:

collecting, from a subject, a liquid specimen sample that includes saliva and an orally-dispensed oral wash fluid;

passing the liquid specimen sample through a filter to trap at least some of the saliva, the filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and

deriving information from extracellular vesicles present in the saliva trapped by the filter.

There is further provided, in accordance with an Inventive Concept 510 of the present invention, a method including:

collecting, from a subject, a liquid specimen sample;

passing the liquid specimen sample through a filter having a bubble-point nominal pore size of between 30 microns and 500 microns, the bubble-point nominal pore size characterized by a bubble point test using a capillary flow porometer and a wetting liquid having a fluid surface tension of 15.9 dynes/cm; and

deriving information from extracellular vesicles present in saliva trapped by the filter.

Inventive Concept 511. The method according to any one of Inventive Concepts 509 and 510, wherein deriving the information includes performing an assay to derive the information.
Inventive Concept 512. The method according to any one of Inventive Concepts 509 and 510, wherein deriving the information includes ascertaining whether the extracellular vesicles present in the specimen sample include a biological marker.
Inventive Concept 513. The method according to any one of Inventive Concepts 509 and 510, wherein the extracellular vesicles are exosomes, and wherein deriving the information includes deriving the information from the exosomes present in the saliva trapped by the filter.
Inventive Concept 514. The method according to any one of Inventive Concepts 509 and 510, wherein the extracellular vesicles are microvesicles, and wherein deriving the information includes deriving the information from the microvesicles present in the saliva trapped by the filter.
Inventive Concept 515. The method according to any one of Inventive Concepts 509 and 510, wherein the extracellular vesicles are apoptotic bodies, and wherein deriving the information includes deriving the information from the apoptotic bodies present in the saliva trapped by the filter.
Inventive Concept 516. The method according to any one of Inventive Concepts 509 and 510, wherein deriving the information from the extracellular vesicles includes identifying one or more proteins of the extracellular vesicles.
Inventive Concept 517. The method according to any one of Inventive Concepts 509 and 510, wherein deriving the information from the extracellular vesicles includes identifying one or more nucleic acids of the extracellular vesicles.
Inventive Concept 518. The method according to any one of Inventive Concepts 509 and 510, wherein the bubble-point nominal pore size is at least 40 microns.
Inventive Concept 519. The method according to Inventive Concept 518, wherein the bubble-point nominal pore size is at least 50 microns.
Inventive Concept 520. The method according to Inventive Concept 519, wherein the bubble-point nominal pore size is at least 60 microns.
Inventive Concept 521. The method according to Inventive Concept 520, wherein the bubble-point nominal pore size is at least 100 microns.
Inventive Concept 522. The method according to Inventive Concept 521, wherein the bubble-point nominal pore size is at least 120 microns.
Inventive Concept 523. The method according to any one of Inventive Concepts 509 and 510, wherein the bubble-point nominal pore size less than 300 microns.
Inventive Concept 524. The method according to Inventive Concept 523, wherein the bubble-point nominal pore size less than 200 microns.
Inventive Concept 525. The method according to any one of Inventive Concepts 509 and 510, wherein the bubble-point nominal pore size is between 50 and 200 microns.
Inventive Concept 526. The method according to Inventive Concept 525, wherein the bubble-point nominal pore size is between 50 and 150 microns.
Inventive Concept 527. The method according to Inventive Concept 526, wherein the bubble-point nominal pore size is between 100 and 150 microns.
Inventive Concept 528. The method according to any one of Inventive Concepts 509 and 510, wherein the method does not include centrifuging the liquid specimen sample.
Inventive Concept 529. The method according to Inventive Concept 509,

wherein the filter is one of a plurality of filters arranged in a series of filters, the filters having respective bubble-point nominal pore sizes, all of which are between 30 microns and 1.5 mm,

wherein passing the liquid specimen sample through the filter includes passing the liquid specimen sample through the series of filters, without passing the liquid specimen sample through any other filters, and

wherein deriving the information from the extracellular vesicles present in the saliva includes deriving the information from the extracellular vesicles present in the saliva trapped by at least one of the plurality of filters.

Inventive Concept 530. The method according to Inventive Concept 529, wherein deriving the information from the extracellular vesicles present in the saliva includes deriving the information from the extracellular vesicles present in the saliva trapped by at least two of the plurality of filters.
Inventive Concept 531. The method according to Inventive Concept 509,

wherein the filter is a first filter, wherein passing the liquid specimen sample through the filter includes passing the liquid specimen sample through the first filter to produce a filtrate, and wherein deriving the information from the extracellular vesicles present in the saliva trapped by the filter includes deriving the information from the extracellular vesicles present in the saliva trapped by the first filter, and

wherein the method further includes:
passing the filtrate through a second filter to trap at least some of the saliva, and
deriving the information from the extracellular vesicles present in the saliva trapped by the second filter.
   Inventive Concept 532. The method according to Inventive Concept 531, wherein a bubble-point nominal pore size of the second filter equals the bubble-point nominal pore size of the first filter.
   Inventive Concept 533. The method according to Inventive Concept 531, wherein a bubble-point nominal pore size of the second filter is between 0.03 and 21 microns.
   Inventive Concept 534. The method according to Inventive Concept 533, wherein the bubble-point nominal pore size of the second filter is between 0.8 and 1.5 microns.
   Inventive Concept 535. The method according to Inventive Concept 509, wherein passing the liquid specimen sample through the filter includes passing the liquid specimen sample through the filter to trap at least some of the saliva by size-based filtration.
   Inventive Concept 536. The method according to Inventive Concept 509, wherein passing the liquid specimen sample through the filter includes passing the liquid specimen sample through the filter to trap at least some of the saliva by adhesion of the saliva to the filter.
   Inventive Concept 537. The method according to any one of Inventive Concepts 509 and 510, wherein the filter is a depth filter.
   Inventive Concept 538. The method according to any one of Inventive Concepts 509 and 510, wherein the filter includes fibers.
   Inventive Concept 539. The method according to Inventive Concept 538, wherein the filter includes crisscrossing fibers.
   Inventive Concept 540. The method according to any one of Inventive Concepts 509 and 510, wherein the filter includes a mesh filter.
   Inventive Concept 541. The method according to Inventive Concept 540, wherein the mesh filter includes a woven mesh filter.
   Inventive Concept 542. The method according to Inventive Concept 540, wherein the mesh filter includes a non-woven mesh filter.
   Inventive Concept 543. The method according to any one of Inventive Concepts 509 and 510, wherein the filter includes a screen.
   Inventive Concept 544. The method according to Inventive Concept 543, wherein the screen includes a woven screen.
   Inventive Concept 545. The method according to Inventive Concept 543, wherein the screen includes a non-woven screen.
   Inventive Concept 546. The method according to any one of Inventive Concepts 509 and 510, wherein the filter includes a polymer.
   Inventive Concept 547. The method according to Inventive Concept 546, wherein the polymer includes a polyester.
   Inventive Concept 548. The method according to any one of Inventive Concepts 509 and 510, wherein passing the liquid specimen sample through the filter includes passing the liquid specimen sample through exactly one filter.
   Inventive Concept 549. The method according to any one of Inventive Concepts 509 and 510, wherein collecting the liquid specimen sample includes receiving the liquid specimen sample by a healthcare worker from the subject.
   Inventive Concept 550. The method according to any one of Inventive Concepts 509 and 510, wherein the method does not include swabbing a nasal cavity of the subject.
   Inventive Concept 551. The method according to any one of Inventive Concepts 509 and 510, wherein the method does not include swabbing a pharynx of the subject.
   Inventive Concept 552. The method according to any one of Inventive Concepts 509 and 510, wherein the method does not include swabbing a nasal cavity of the subject and does not include swabbing a pharynx of the subject.
   Inventive Concept 553. The method according to any one of Inventive Concepts 509 and 510, wherein the method does not include swabbing any portion of the subject.
   Inventive Concept 554. The method according to any one of Inventive Concepts 509 and 510, wherein collecting the liquid specimen sample includes collecting the liquid specimen sample that was expressed via an anterior opening of an oral cavity of the subject.
   Inventive Concept 555. The method according to any one of Inventive Concepts 509 and 510, wherein collecting the liquid specimen sample includes aspirating the liquid specimen sample via an anterior opening of an oral cavity of the subject.
   Inventive Concept 556. The method according to Inventive Concept 509, wherein collecting the liquid specimen sample includes:

gargling, by the subject, the oral wash fluid to produce the liquid specimen sample that includes the oral wash fluid and the saliva; and

thereafter, collecting the liquid specimen sample from the subject.

Inventive Concept 557. The method according to Inventive Concept 509, wherein collecting the liquid specimen sample includes:

gargling, by the subject, the oral wash fluid to produce the liquid specimen sample that includes the oral wash fluid and the saliva; and

thereafter, expressing, by the subject, out of an anterior opening of an oral cavity of the subject, the liquid specimen sample.

Inventive Concept 558. The method according to Inventive Concept 509, wherein collecting the liquid specimen sample includes:

swishing, by the subject, the oral wash fluid in an oral cavity of the subject to produce the liquid specimen sample that includes the oral wash fluid and the saliva; and

thereafter, collecting the liquid specimen sample from the subject.

Inventive Concept 559. The method according to Inventive Concept 509, wherein collecting the liquid specimen sample includes:

swishing, by the subject, the oral wash fluid in an oral cavity of the subject to produce the liquid specimen sample that includes the oral wash fluid and the saliva; and

thereafter, expressing, by the subject, out of an anterior opening of the oral cavity, the liquid specimen sample.

Inventive Concept 560. The method according to any one of Inventive Concepts 509 and 510, wherein collecting the liquid specimen sample includes:

spraying, via an anterior opening of an oral cavity of the subject, an oropharynx of the subject with the oral wash fluid; and

thereafter, collecting the liquid specimen sample from the subject.

Inventive Concept 561. The method according to Inventive Concept 509, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, collecting the liquid specimen sample that passed out of an anterior opening of an oral cavity of the subject and includes the oral wash fluid, the nasal wash fluid, and at least a portion of the biological material washed into the oropharynx by the nasal wash fluid.

Inventive Concept 562. The method according to Inventive Concept 561, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

collecting the liquid specimen sample that passed out of the anterior opening of the oral cavity.

Inventive Concept 563. The method according to Inventive Concept 561, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

collecting the liquid specimen sample that passed out of the anterior opening of the oral cavity.

Inventive Concept 564. The method according to Inventive Concept 509, wherein collecting the liquid specimen sample from the subject includes:

intranasally dispensing nasal wash fluid into the nasal cavity such that the nasal wash fluid washes biological material into an oropharynx of the subject from (a) the nasal cavity, (b) a nasopharynx of the subject, or (c) the nasal cavity and the nasopharynx; and

thereafter, expressing, by the subject, out of an anterior opening of an oral cavity of the subject, the liquid specimen sample that includes the oral wash fluid, the nasal wash fluid, and at least a portion of the biological material washed into the oropharynx by the nasal wash fluid.

Inventive Concept 565. The method according to Inventive Concept 564, wherein collecting the liquid specimen sample includes:

after intranasally dispensing the nasal wash fluid, orally dispensing the oral wash fluid into the oral cavity via the anterior opening of the oral cavity such that the oral wash fluid forms a mixture with the nasal wash fluid in the oropharynx;

gargling, by the subject, the mixture to produce the liquid specimen sample that includes the oral wash fluid, the nasal wash fluid, and the saliva; and

expressing, by the subject, the liquid specimen sample out of the anterior opening of the oral cavity.

Inventive Concept 566. The method according to Inventive Concept 564, wherein collecting the liquid specimen sample includes:

after intranasally dispensing the nasal wash fluid, orally dispensing the oral wash fluid into the oral cavity via the anterior opening of the oral cavity;

gargling, by the subject, the oral wash fluid to produce the liquid specimen sample that includes the oral wash fluid, the nasal wash fluid, and the saliva; and

expressing, by the subject, the liquid specimen sample out of the anterior opening of the oral cavity.

Inventive Concept 567. The method according to any one of Inventive Concepts 561 and 564, wherein intranasally dispensing includes intranasally dispensing, by the subject, the nasal wash fluid into the nasal cavity.
Inventive Concept 568. The method according to any one of Inventive Concepts 561 and 564, wherein intranasally dispensing includes intranasally dispensing, by a healthcare worker, the nasal wash fluid into the nasal cavity.

There is still further provided, in accordance with an Inventive Concept 569 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx;

thereafter, collecting a specimen sample that passed out of an anterior opening of an oral cavity of the subject and contains at least a portion of the biological material washed into the oropharynx by the nasal wash fluid; and

diagnosing cancer based at least in part on information derived from the specimen sample.

Inventive Concept 570. The method according to Inventive Concept 569, wherein diagnosing the cancer includes screening the specimen sample for cells that are possibly cancerous or precancerous.

There is additionally provided, in accordance with an Inventive Concept 571 of the present invention, a method including:

intranasally dispensing nasal wash fluid into a nasal cavity of a subject such that the nasal wash fluid washes biological material into an oropharynx of the subject from (a) the nasal cavity, (b) a nasopharynx of the subject, or (c) the nasal cavity and the nasopharynx;

thereafter, collecting a specimen sample that passed out of an anterior opening of an oral cavity of the subject and contains at least a portion of the biological material washed into the oropharynx by the nasal wash fluid; and

monitoring cancer by testing the specimen sample.

The present invention will be understood from the following detailed description of embodiments thereof, taken together with the drawings, in which:

### BRIEF DESCRIPTION OF THE DRAWINGS

FIGS. 1A-B are schematic illustrations of a method for collecting a specimen sample from a subject, in accordance with an application of the present invention;

FIG. 2 is a schematic illustration of a testing kit, in accordance with an application of the present invention; and

FIG. 3 is a schematic illustration of a nasal wash fluid dispenser, in accordance with an application of the present invention;

FIGS. 4A-D are schematic cross-sectional illustrations of a method of collecting a specimen sample from a subject, in accordance with an application of the present invention;

FIGS. 5A-B are schematic cross-sectional illustrations of the nasal wash fluid dispenser of FIG. 3 inserted into a nasal cavity, in accordance with an application of the present invention;

FIG. 6 is a schematic cross-sectional illustration of a portion of a method for collecting a specimen sample from the subject, in accordance with an application of the present invention;

FIGS. 7A-B are schematic illustrations of a portion of a method for collecting a specimen sample from a subject, in accordance with an application of the present invention; and

FIGS. 8A-B are schematic illustrations of filter units for testing a liquid specimen sample that includes saliva, in accordance with respective applications of the present invention.

### DETAILED DESCRIPTION OF APPLICATIONS

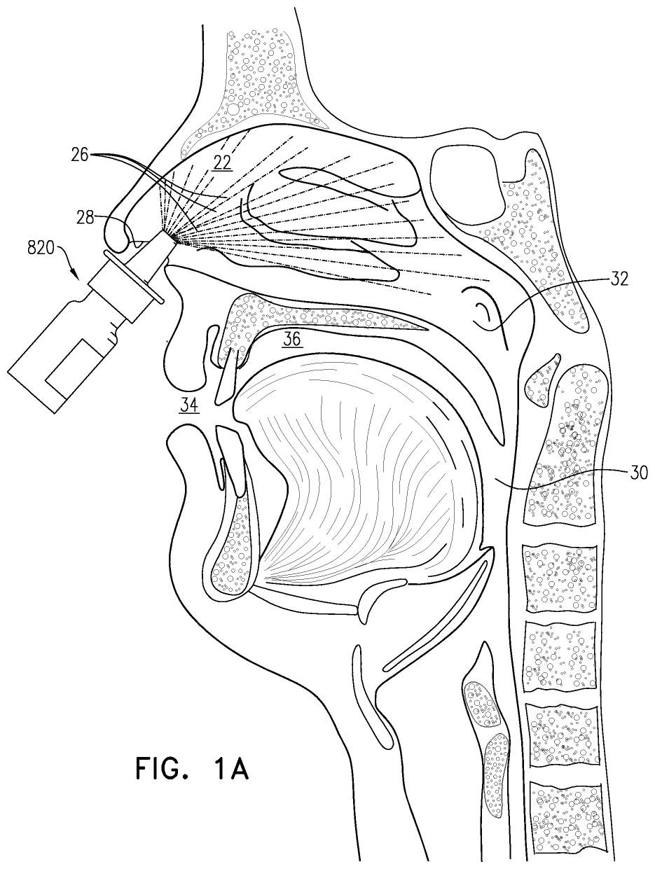

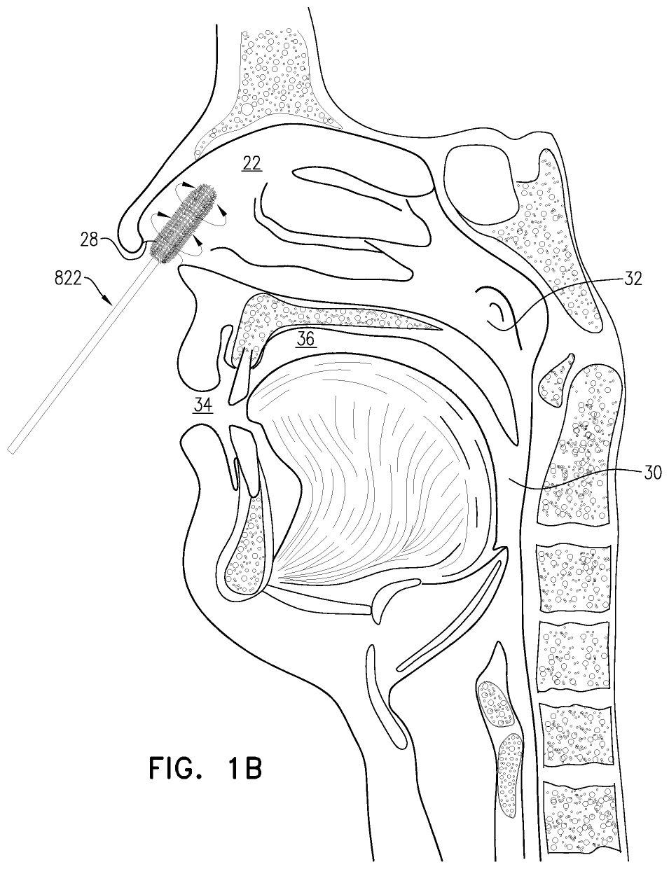

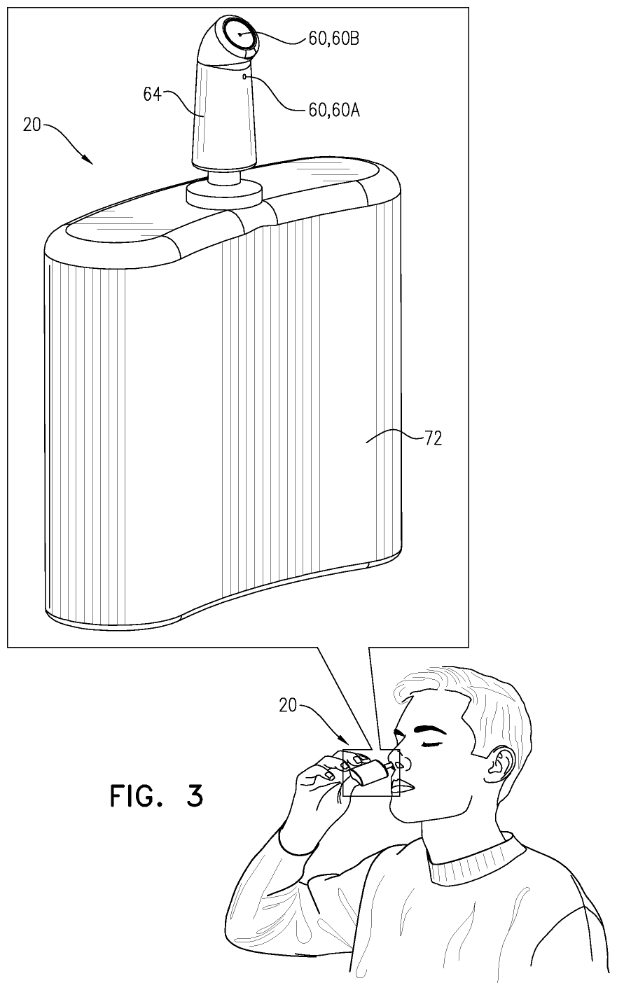

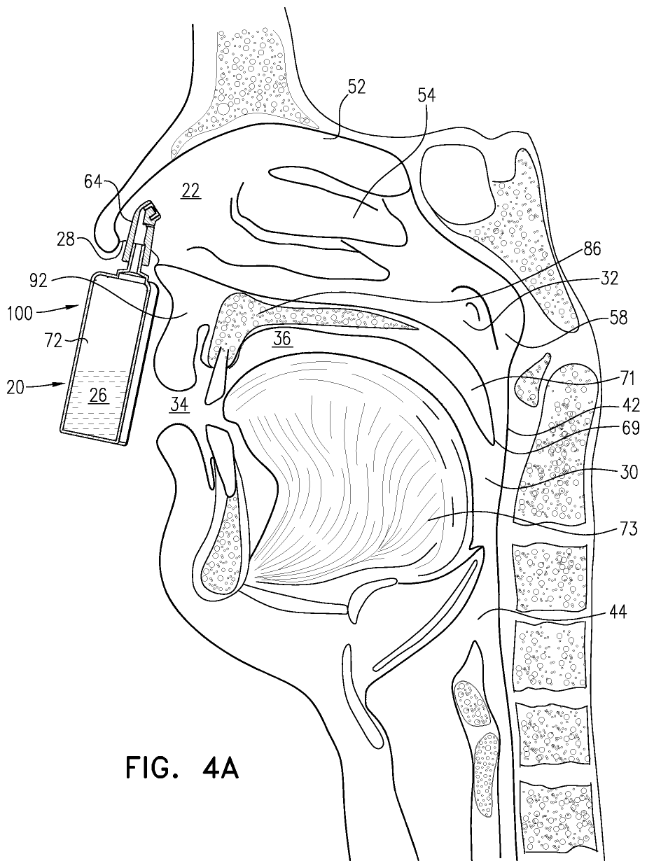

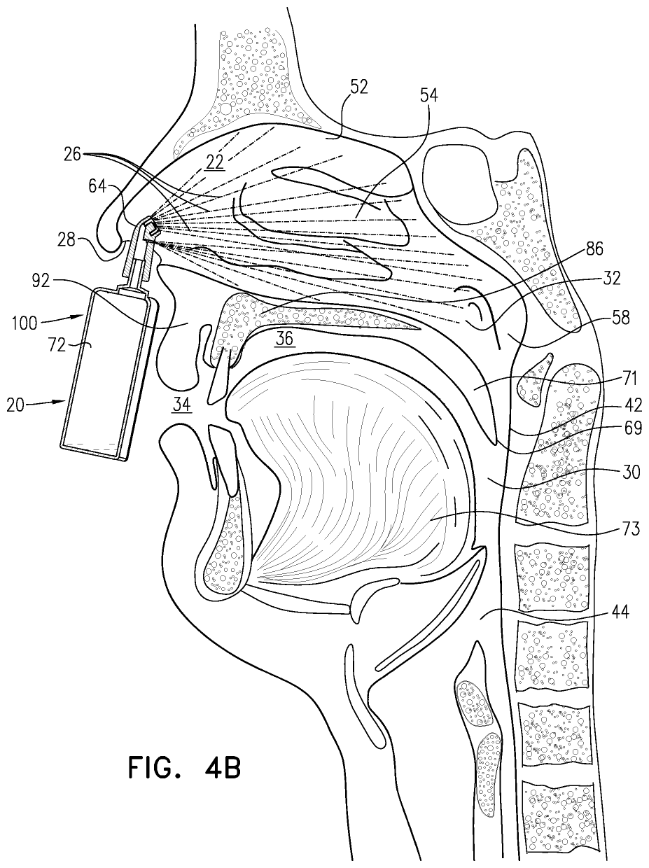

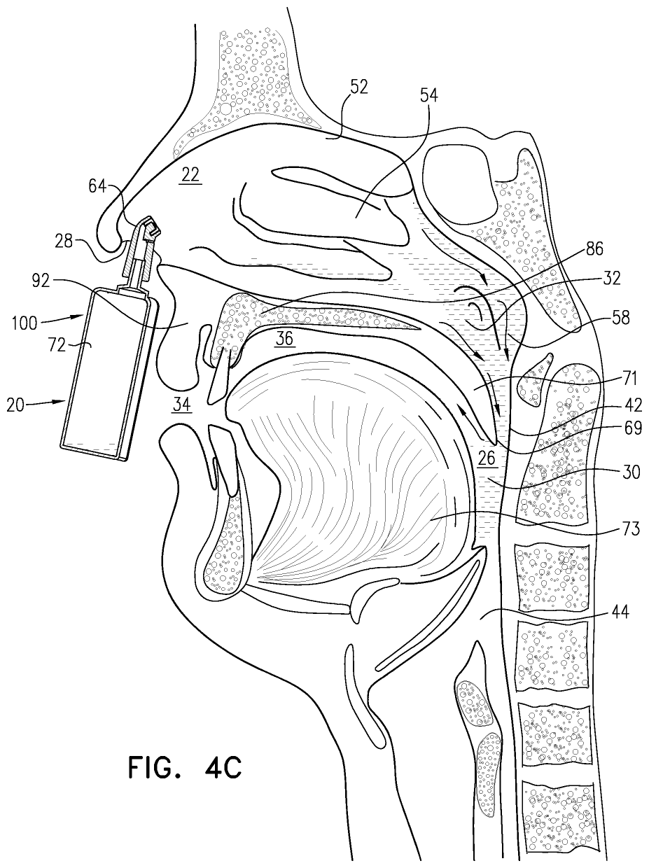

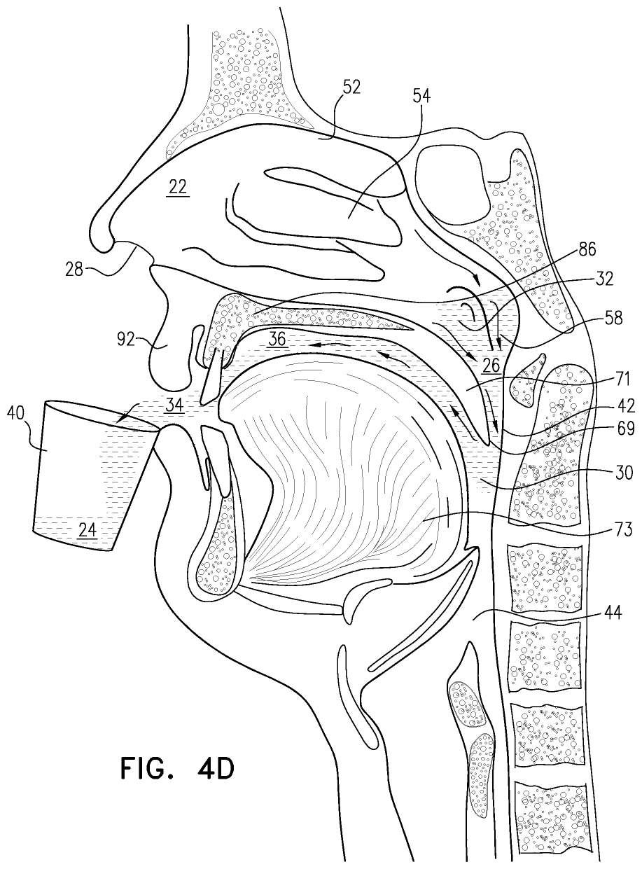

Reference is now made to FIGS. 1A-B, which are schematic illustrations of a method for collecting a specimen sample from a subject, in accordance with an application of the present invention. The method comprises intranasally dispensing nasal wash fluid 26 into a nasal cavity 22, by the subject or by somebody else. Nasal wash fluid 26 may be intranasally dispensed using a conventional nasal dispenser 820, such as shown in FIG. 1A; using a nasal wash fluid dispenser 20, such as shown in FIGS. 3-4C, described hereinbelow; using a nasal sampling device 1020, such as shown in FIG. 22 of U.S. application Ser. No. 17/921,672, which is assigned to the assignee of the present application and incorporated herein by reference, and is the US national stage of International Application PCT/IL2021/050519, filed May 6, 2021, which published as PCT Publication WO 2021/224925 and is assigned to the assignee of the present application and incorporated herein by reference; or using any nasal wash fluid dispenser known in the art. Nasal wash fluid 26 generally loosens biological material in nasal cavity 22 and/or a nasopharynx 32. Thereafter, a specimen sample is collected by performing, by the subject or by somebody else, one or more nasal swabs selected from the group consisting of: an anterior nares swab (such as shown in FIG. 1B) and a mid-turbinate swab (not shown). The nasal swab may be performed using a conventional nasal swab 822, such as shown in FIG. 1B, or using nasal sampling device 1020, such as shown in FIG. 22 of the '519 PCT application. The specimen sample is typically collected without performing a nasopharyngeal swab. When the specimen sample is collected by performing the anterior nares swab, the specimen sample is optionally collected without performing a mid-turbinate swab or a nasopharyngeal swab. The nasal swab may be performed in one or both of the nostrils; in the latter case, nasal wash fluid 26 is intranasally dispensed into both nostrils of nasal cavity 22, and the same single swab is typically used in both nostrils. Alternatively or additionally, the specimen sample is collected by performing a nasopharyngeal swab.

In some applications of the present invention, a method is provided that comprises intranasally dispensing nasal wash fluid 26 into nasal cavity 22 of a subject, by the subject or somebody else, such as shown in FIG. 1A. Nasal wash fluid 26 generally loosens biological material in nasal cavity 22 and/or nasopharynx 32. Thereafter, a specimen sampler is inserted into nasal cavity 22 by the subject or by somebody else, such that a farthest-inserted portion of the specimen sampler is positioned in an anterior naris of nasal cavity 22. A specimen sample is collected on the specimen sampler by contacting (e.g., rubbing) a wall of the anterior naris (i.e., the wall's epithelial surface) with the specimen sampler, optionally including rotating the specimen sampler. For example, the specimen sampler may comprise a swab (e.g., a flocked swab) (such as shown in FIG. 1B), a brush, a sponge, or a nasal plug. Typically, but not necessarily, the specimen sampler comprises a tip comprising an absorbent material. For applications in which the specimen sampler comprises a swab, a tip of the swab may, for example, be a flocked swab tip (e.g., comprising nylon), a rayon swab tip, a cotton swab tip, a polyurethane foam swab tip, a polyethylene terephthalate (PET) swab tip, a polyester swab tip, a foam swab tip (e.g., comprising polyurethane) (e.g., an open-cell foam swab), a sponge swab tip (e.g., comprising cellulose), a brush swab tip (e.g., similar to a cytology brush), and a nanofiber swab tip. The specimen sampler may be inserted into a single nostril of nasal cavity 22, or into both of the nostrils, one at a time; in the latter case, nasal wash fluid 26 is intranasally dispensed into both nostrils of nasal cavity 22.

In some applications of the present invention, a method is provided that comprises intranasally dispensing nasal wash fluid 26 into nasal cavity 22 of a subject, by the subject or somebody else, such as shown in FIG. 1A. Nasal wash fluid 26 generally loosens biological material in nasal cavity 22 and/or nasopharynx 32. Thereafter, a specimen sampler is inserted into nasal cavity 22 by the subject or by somebody else. While the specimen sample is inserted into the nasal cavity, a specimen sample is collected on the specimen sampler without using suction, such as shown in FIG. 1B.

In some applications of the present invention, a method is provided that comprises intranasally dispensing nasal wash fluid 26 into nasal cavity 22 of a subject, by the subject or somebody else, such as shown in FIG. 1A. Nasal wash fluid 26 generally loosens biological material in nasal cavity 22 and/or nasopharynx 32. Thereafter, the subject or somebody else inserts, into nasal cavity 22, a specimen sampler that is shaped so as not to define any dispensing openings. While the specimen sampler is inserted into the nasal cavity, a specimen sample is collected on the specimen sampler, such as shown in FIG. 1B.

For some applications, before the specimen sample is collected using one of the techniques described in the previous paragraphs (by performing the nasal swab or using a specimen sampler inserted into the nasal cavity):
before nasal wash fluid 26 is intranasally dispensed, the subject (typically while sitting or standing) tilts back his or her head (typically until the neck is fully extended) and holds the head tilted back while nasal wash fluid 26 is intranasally dispensed, or
after nasal wash fluid 26 is intranasally dispensed, the subject tilts his or her head farther back than while intranasally dispensing the nasal wash fluid and farther back than a neutral position.

In all of these cases, the tilting back may cause nasal wash fluid 26 to loosen biological material in a more posterior (deeper) portion of nasal cavity 22 and/or nasopharynx 32.

Optionally, the subject sniffs after nasal wash fluid 26 is intranasally dispensed and before the specimen sample is collected. In applications in which the subject tilts the head far back, the sniffing may be performed before or after such far back tilting.

For some applications, nasal wash fluid 26 is intranasally dispensed without blowing, by the subject, a nose of the subject immediately prior to intranasally dispensing the nasal wash fluid. Blowing the nose may undesirably remove mucus and other biological material containing high concentrations of particulate of interest. Alternatively, the subject may blow his or her nose before the nasal wash fluid is intranasally dispensed.

Optionally, after the subject tilts his or her head back and after nasal wash fluid 26 is intranasally dispensed, the subject tilts forward his or her head to a neutral or a tilted-forward position; this backward-followed-by-forward tilting may help move biological material anteriorly (i.e., toward the nostril) within nasal cavity 22.

Optionally, the method further comprises, while holding his or her head tilted back, waiting for a period of time between (a) concluding intranasally dispensing nasal wash fluid 26 into the nasal cavity or tilting the head farther back and (b) tilting his or her head forward to the neutral or the tilted-forward position, the period of time at least 2 seconds (e.g., at least 5 seconds, such as at least 10 seconds, e.g., at least 15 seconds), no more than 60 seconds (e.g., no more than 30 seconds), and/or between 2 seconds (e.g., 5 seconds, such as 10 seconds, e.g., at least 15 seconds) and 60 seconds (e.g., 30 seconds).

Optionally, while holding his or her head tilted back, the subject makes a sound, such as a “K” sound, that inhibits flow of nasal wash fluid 26 from nasopharynx 32 to oropharynx 30, helping retain nasal wash fluid 26 in nasopharynx 32.

For some applications, the subject tilts his or her head forward to the neutral or the tilted-forward position after the subject senses (e.g., by feeling and/or tasting) the nasal wash fluid in oropharynx 30. Optionally, nasal wash fluid 26 comprises a tastant, for example, menthol flavoring.

For some applications, after tilting the head forward to the neutral or the tilted-forward position, the subject waits for a period of time before collecting the specimen sample, the period of time at least 5 seconds, e.g., at least 10 seconds, such as at least 30 seconds (and typically less than 120 seconds). This waiting period may allow sufficient time for nasal wash fluid 26 to drip anteriorly in nasal cavity 22, bringing the biological material with it.

Alternatively, for some applications, nasal wash fluid 26 is intranasally dispensed into nasal cavity 22 (either continuously or in a plurality of pulses) until the subject senses (e.g., by feeling and/or tasting) nasal wash fluid 26 in oropharynx 30. Optionally, nasal wash fluid 26 comprises a tastant, for example, menthol flavoring.

Optionally, the method further comprises waiting for a period of time between (a) concluding intranasally dispensing nasal wash fluid 26 into the nasal cavity and (b) collecting the specimen sample, the period of time at least 2 seconds (e.g., at least 5 seconds, such as at least 10 seconds), no more than 30 minutes (e.g., no more than 15 minutes), and/or between 2 seconds (e.g., 5 seconds, such as at least 10 seconds) and 30 minutes (e.g., 15 minutes).

For some applications, nasal wash fluid 26 is intranasally dispensed as one or more types of fluid discharge selected from the group consisting of: a spray, a mist, a pressurized aerosol, steam, and an atomized fluid. For some applications, nasal wash fluid 26 is intranasally dispensed as a fluid stream.

For some applications, nasal wash fluid 26 is dispensed into each of one or both nostrils 28 in a total volume of between 0.15 and 1 mL, such as between 0.15 and 0.5 mL, or between 0.25 and 1 mL, such as between 0.25 and 0.5 mL. Optionally, nasal wash fluid 26 is intranasally dispensed in a plurality of pulses per nostril (e.g., between 3 and 5 pulses per nostril), each having a volume of between 0.05 and 0.2 mL, for example, between 0.05 and 0.15 mL, e.g., 0.1 mL. (If nasal wash fluid 26 is dispensed into both nostrils 28, the volumes above would be doubled.) Dispensing such relatively small volumes reduces the likelihood of nasal wash fluid 26 washing out the biological material of interest from the nostrils.

In some applications, the specimen samples collected using the collection techniques described herein are tested for a particulate using one or more of the techniques described herein, one or more of the techniques described in the patent applications incorporated hereinbelow by reference, or a technique known in the art. The particulate may be a virus (e.g., SARS-CoV-2 or an Influenza virus), a bacterium, any of the other particulates described hereinabove, or any of the other biological materials described hereinabove. For example, the particulate may be tested for the presence of a particulate by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, the particulate may be tested the presence of a particulate using a CRISPR-based diagnostic test, an ELISA diagnostic test, or a spectroscopy-based diagnostic test.

These sampling methods may be combined with any other applicable techniques described herein, mutatis mutandis, and/or in the patent applications incorporated herein by reference. By way of example and not limitation, these sampling methods may further comprise, after nasal wash fluid 26 is intranasally dispensed, orally dispensing oral wash fluid 68 into an oral cavity 36 via an anterior opening 34 of oral cavity 36; and gargling, by the subject, oral wash fluid 68. Optionally:
oral wash fluid is orally dispensed into oral cavity 36 such that the oral wash fluid forms a mixture with nasal wash fluid 26 in an oropharynx 30, and, in addition to collecting the specimen sample from the nasal cavity (by swabbing or using the specimen sampler), a specimen sample 24 is collected that passed out of anterior opening 34 of oral cavity 36 and contains at least a portion of the mixture and, typically, at least a portion of biological material washed into oropharynx 30 with nasal wash fluid 26; and/or
oral wash fluid is orally dispensed into oral cavity 36, the subject gargles the oral wash fluid, such that the oral wash fluid forms a mixture with nasal wash fluid 26, and a specimen sample 24 is collected that passed out of anterior opening 34 of oral cavity 36 and contains at least a portion of the mixture and the at least a portion of biological material washed into oropharynx 30 with nasal wash fluid 26.

Alternatively, a specimen sample is not collected that passed out of anterior opening 34 of oral cavity 36, and the gargled fluid is instead spit out and discarded. In this case, the time spent gargling (e.g., 5-15 seconds) may serve to ensure that the subject holds his or her head tilted back for a sufficient period of time for nasal wash fluid 26 to loosen biological material in nasal cavity 22 (such as in a more posterior (deeper) portion of nasal cavity 22 and/or nasopharynx 32). The gargling may alternatively or additionally distract the subject from the unpleasant sensation of post-nasal drip of nasal wash fluid 26 into the throat, so that the subject does not prematurely tilt his or her head forward to the neutral or the tilted-forward position before nasal wash fluid 26 sufficiently loosens the biological material in nasal cavity 22.

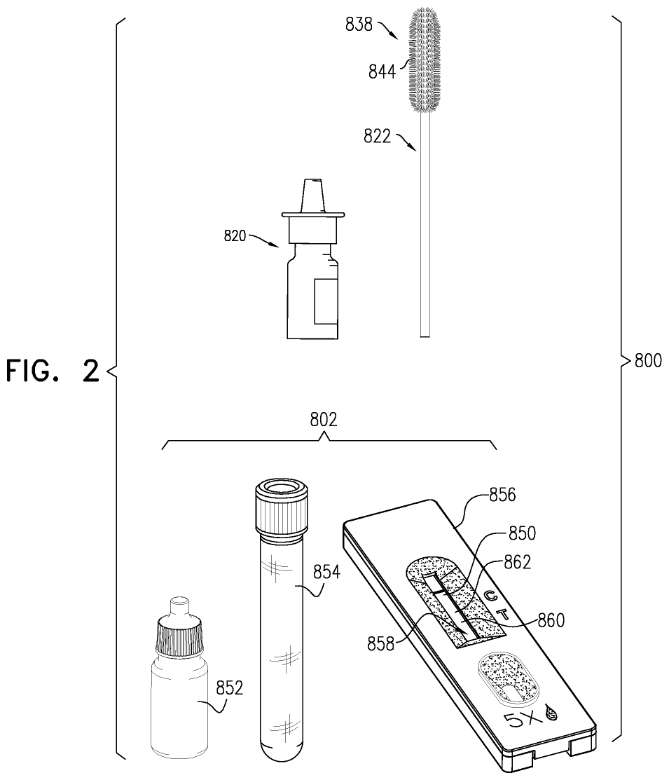

Also by way of example and not limitation, these nasal sampling methods may be performed using any of nasal wash fluid 26 dispensers described in the '519 PCT application with reference to FIGS. 1, 2A-C, 3A-B, 6, 7A-K, 8A-C, 9A-F, 10, and/or 11. Alternatively, these nasal sampling methods may be performed using a conventional nasal dispenser, such as shown in FIG. 1A. Further alternatively, these nasal sampling methods may be performed using nasal sampling device 1020, described in the '519 PCT application with reference to FIGS. 20A-B and 21A-B.

In some applications, nasal wash fluid 26 comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution.

Typically, nasal wash fluid 26 does not comprise a drug molecule. Alternatively, nasal wash fluid 26 does comprise a drug molecule.

Further alternatively or additionally, in some applications, nasal wash fluid 26 comprises a chemical that increases the amount of biological material in specimen sample 24 by enhancing the clearance of the mucus as well as dislodging the biological material lining the mucosa, for example, a mucolytic agent such as acetylcysteine, or for example, a detergent such as polysorbate, or for example, a gas such as pressurized carbon dioxide to generate bubbles or nitrous oxide to increases the speed of mucociliary clearance.

Experiments 1, 2, 4, and 7, described hereinbelow, provide evidence of the efficacy of some of these nasal-spray and nasal-sampling techniques for collecting specimen samples for detection of SARS-CoV-2 using quantitative polymerase chain reaction (PCR) (qPCR).

Experiments 3, 5, 6, and 8, described hereinbelow, provide evidence of the efficacy of some of these nasal-spray and nasal-sampling techniques for collecting specimen samples for detection of SARS-CoV-2 using lateral flow immunoassay strips. As demonstrated in these experiments, intranasally dispensing nasal wash fluid 26 into nasal cavity 22 strengthens an intensity of a test band of the lateral flow immunoassay test strip when the virus is present in the anterior naris. (As is known in the art, lateral flow immunoassay test strips include test bands (also called test lines) and control bands (also called control lines).) In general, test bands having a stronger intensity can be more readily detected, whether by a human eye or by an image sensor.

The inventors believe that one of ordinary skill in the art would not expect that intranasal dispensation of nasal wash fluid 26 would enhance the sensitivity of detection of SARS-CoV-2 (or any other virus or biological particulate) using a lateral flow immunoassay strip, because of the much lower sensitivity of lateral flow immunoassay strips than nucleic acid amplification tests, such as PCR. Lateral flow immunoassay strips are well known to provide accurate results only when a fairly dry and concentrated sample is immersed in a small volume of reagent liquid, such as 0.2 to 0.4 mL, before application to the strip. In addition, anterior nares samples are known to be weaker than mid-turbinate and nasopharyngeal samples. Therefore, one of ordinary skill in the art would expect that the intranasal dispensation of nasal wash fluid 26 might dilute the biological material captured in the anterior nares specimen sample. In addition, one of ordinary skill in the art would expect that the intranasal dispensation of nasal wash fluid 26 might wash biological material potentially containing the virus off of the wall of the anterior nares before collection of the specimen sample, thereby reducing the amount of virus collected from the anterior nares.

For all of these reasons, one of ordinary skill in the art would expect the intranasal dispensation of nasal wash fluid before collecting the specimen sample would reduce the sensitivity of tests performed using lateral flow immunoassay strips. By contrast, the inventors have found that intranasal dispensation of nasal wash fluid 26 improves the sensitivity of lateral flow immunoassay tests performed on nasal samples collected from the anterior nares.

The dimensions and absorptive properties of swabs that may be useful in the nasal swabbing sample collection techniques described herein, such as for testing using a lateral flow immunoassay strip (e.g., for SARS-CoV-2 or an Influenza virus) were examined on behalf of the inventors. Specifically, flocked nasal swabs (3BY Ltd., Industrial Park Tefen, Israel, Ref: 3BY-001) were examined, because they were used in a number of the experiments described herein, and foam tipped nasal swabs (Puritan, Maine, USA, Ref: 25-1506-1PF 100), which were provided with the BD Veritor™ system for Rapid Detection of SARS-CoV-2 (Becton Dickinson and Company, Maryland, USA, Ref: 256082), were examined because they are appropriate for sample collection for use in immunoassays.

Swab tip dimensions were determined using a mechanical caliper, or were provided by swab manufacturers (Table 1). Following nasal saline spraying and swabbing, swabs were found to contain between approximately 76-131 microliters of fluid from a single nostril (Table 2), after administering about 500 microliters of nasal wash into the nostril. Utilizing relatively smaller swab tips for respiratory specimen collection may be beneficial because of the limited volumes of lysing buffer included in commercial immunoassay kits (Table 3), which must be sufficient for the extraction of viral antigens from virions, intact infected cells, or infected cell debris which have been collected by the swab. If swabs were to contain a larger sample volume, the lysing buffer would be diluted beyond the optimal threshold and effective extraction would be unlikely to occur. Moreover, oversized swabs which absorb more fluid than what is recoverable during sampling would further absorb lysing buffer, making it difficult to elute sufficient lysate from the swab for assaying, because standard immunoassay cassettes require 3 drops of lysate (approximately 150 microliters), with dipstick style immunoassays typically absorbing at least 20 microliters of lysate.

TABLE 1
Swab Dimensions

| Swab Type | Flocked | Foam |
|---|---|---|
| Greatest Tip Diameter (mm)^{v} | 4 | 4.775ⁱ |
| Tip Length (mm) | 17 | 16.002ⁱ |
| Tip Surface Area (mm²)ⁱⁱ | 226 | 258 |
| Tip Volume (mm³)ⁱⁱⁱ | 214 | 286 |
| Applicator Thickness at Tip (mm) | 2 | 2.54 ⁱ |
| Applicator Length at Tip (mm) | 16.5 | 13 |
| Applicator Volume at Tip (mm2)ⁱⁱⁱ | 52 | 64 |
| Effective Absorptive Volume (mm³)^{iii, iv} | 162 | 222 |

i—Dimensions provided by manufacturer (Puritan Medical Products Company LLC, Guilford, Me., USA)
ii—Surface area was calculated assuming a perfectly cylindrical shape but excluding the bottom of the tip because of the presence of the applicator (2πrh+πr\hat{ }2).
iii—Volume was calculated assuming a perfectly cylindrical shape.
iv—Effective absorptive volume was determined by subtracting the applicator volume at tip from the tip volume.
iv—The greatest tip diameter is the diameter of the swab at the widest location along the swab.

TABLE 2
Swab Absorption

| | | Weight | Weight | | |
|---|---|---|---|---|---|
| | | Before | After | | Fluid |
| Swabbing | | Swabbing | Swabbing | Difference | Volume |
| Type | Swab | (g) | (g) | (g) | (microliters)ⁱ |
| Saturation | Flocked | 0.668 | 0.768 | 0.1 | 100 |
| with DPBS | Foam | 0.468 | 0.502 | 0.034 | 34 |
| (−Rotation)ⁱⁱ | | | | | |
| Saturation | Flocked | 0.691 | 0.795 | 0.104 | 104 |
| with DPBS | Foam | 0.476 | 0.651 | 0.175 | 175 |
| (+Rotation)ⁱⁱ | | | | | |
| Bilateral | Flocked | 0.669 | 0.672 | 0.003 | 3 |
| Swabbing | Foam | 0.497 | 0.493 | −0.004 | −4^{V} |
| Spraying and | Flocked | 0.672 | 0.768 | 0.096 | 96 |
| Unilateral | Foam | 0.472 | 0.548 | 0.076 | 76 |
| Swabbingⁱⁱⁱ | | | | | |
| Spraying and | Flocked | 0.678 | 0.783 | 0.105 | 105 |
| Bilateral | Foam | 0.469 | 0.6 | 0.131 | 131 |
| Swabbingⁱⁱⁱ | | | | | |

i—Assumed density of 1 g/cm3 for absorbed liquid.
ii—Swabs were dipped into a 50 mL conical tube containing Dulbecco's Phosphate Buffered Saline (DBPS), and them either removed (− rotation) or rotated while brushing the walls of the tube five times to simulate anterior nares swabbing (+ rotation)
iii—Unilateral and Bilateral swabbing of the anterior nares were performed by inserting the entire tip of the swab into the nares and rotating the swab around the inner walls of the nares. Nasal saline spraying consisted of 5 sprays of 0.1 mL of saline into one or both nostrils.
iv—Negative values are assumed to be a result of either a small amount of the tip separating from the swab during swabbing, or are an artifact due to inaccuracies in the analytical balance.

TABLE 3
Lysing Buffer Volumes

| | | Weight of | Approximate |
|---|---|---|---|
| | | Lysing | Volume |
| Assay Name | Manufacturer | Buffer (g) | (microliters)ⁱ |
| QuickVue Influenza | Quidel Corporation | 0.245 | 245 |
| A + B Test | (San Diego, CA, | | |
| | Ref: 20183) | | |
| BD Veritor ™ | Becton Dickinson | 0.413 | 413 |
| system for Rapid | and Company | | |
| Detection of SARS- | (Maryland, USA, | | |
| CoV-2 | Ref: 256082) | | |
| COVID-19 Antigen | Beijing Kewei | 0.283 | 283 |
| Rapid Test Cassette | Clinical Diagnostic | | |
| | Reagent Inc | | |
| | (Beijing, China, | | |
| | Ref: 601450) | | |

i—Assumed density of 1 g/cm3 for liquid reagents

For some applications, any of the swabs used in the methods described herein, or as elements of apparatus described herein, comprise a tip having one or more of the following characteristics:
a greatest diameter of at least 2 mm, less than 5 mm (e.g., less than 4.5 mm), and/or between 2 and 5 mm (e.g., between 2 and 4.5 mm),
a collection surface area of at least 200 mm2, less than 300 mm2, and/or between 200 and 300 mm2 (the collection surface area includes the absorptive portion of the swab, but excludes the bottom of the tip because of the presence of the applicator),
a volume of at least 200 mm3, less than 300 mm3, and/or between 200 and 300 mm3, and/or
a length of at least 12 mm, less than 20 mm, and/or between 12 and 20 mm.

Optionally, any of the swabs and/or specimen samplers described herein, including, but not limited to, with reference to FIG. 2, may have the characteristics described immediately above.

For some applications, in any of the nasal collection techniques described herein, collecting the specimen sample comprises collecting at least 25 microliters, less than 150 microliters (e.g., less than 125 microliters), and/or between 25 and 150 microliters (e.g., between 25 and 125 microliters) of material per specimen sampler, e.g., per swab (such as, for example, flocked swab). The material typically includes a combination of the biological material of interest and a portion of nasal wash fluid 26 dispensed into nasal cavity 22. Alternatively, for some applications, in any of the nasal collection techniques described herein, collecting the specimen sample comprises collecting at least 25 microliters, less than 250 microliters (e.g., less than 200 microliters), and/or between 25 and 250 microliters (e.g., between 25 and 200 microliters) of material per specimen sampler, e.g., per swab (such as, for example, foam swab).

In some applications of the present invention, a method is provided that comprises intranasally dispensing nasal wash fluid 26 into nasal cavity 22 of a subject, by the subject or by somebody else. Nasal wash fluid 26 generally loosens biological material in nasal cavity 22 and/or nasopharynx 32. Thereafter, a specimen sample is collected by blowing a nose, by the subject, into a specimen receptacle. These techniques may be practiced in combination with any of the techniques described herein for intranasally dispensing nasal wash fluid 26 into nasal cavity 22.

Reference is now made to FIG. 2, which is a schematic illustration of a testing kit 800, in accordance with an application of the present invention. Testing kit 800 comprises:
a nasal dispenser (containing nasal wash fluid 26), such as conventional nasal dispenser 820, as shown, or nasal wash fluid dispenser 20, not shown in FIG. 2, but shown in FIGS. 3-4C;
conventional nasal swab 822; and
a test 802 (e.g., a diagnostic test), which is configured to test for the presence of a particulate in a specimen sample collected using the techniques described herein.

Test 802 may optionally be configured to implement testing techniques described herein, in the patent applications incorporated hereinbelow by reference, and/or known in the art. The particulate may be a virus, a bacterium, any of the other particulates described hereinabove, or any of the other biological materials described hereinabove.

For some applications, test 802 comprises a lateral flow immunoassay test strip 850, which is configured to detect the presence of the particulate (such as by detecting protein antigen, e.g., from a virus). Test 802 typically further comprises one or more reagents 852, such as extraction reagents, as known in the art. Lateral flow immunoassay test strips are well known in the art. For example, they typically contain an antibody specific to an antigen, and the specimen sample fluid migrates up the test strip and reacts with the antibody, thus generating a line on the test strip; the presence of this line indicates a positive test result.

Test 802 may optionally further comprise an extraction tube 854, to aid in bathing a swab tip 838 of nasal swab 822 in the one or more reagents 852 after several drops of the reagents have been inserted into the extraction tube, as is known in the art.

Lateral flow immunoassay test strip 850 may optionally be enclosed in a plastic cassette 856 (such as shown) or a card (configuration not shown), both as known in the art, or may alternatively be unenclosed (configuration not shown). The cassette or card typically is shaped so as to define a window 858 that provides a view of a test band 860 and a control band 862, as known in the art (the bands are not visible in FIG. 2 because the test sample and reagents have not yet been applied to the test strip).

For example, lateral flow immunoassay test strip 850 may be implemented using commercially available COVID-19 antigen tests available from Quidel Corporation (San Diego, Calif., USA), such as the QuickVue® At-Home OTC COVID-19 test; or available from Abbott Laboratories (Abbott Park, Ill., USA), such as the BinaxNOW COVID-19 Ag Card rapid antigen test or the Panbio™ COVID-19 Ag Rapid Test Device.

For some applications, lateral flow immunoassay test strip 850 is an element of a chromatographic digital immunoassay, such as, for example, the BD Veritor™ system for Rapid Detection of SARS-CoV-2 (Becton, Dickinson and Company, Maryland, USA, Ref: 256082), which is a chromatographic digital immunoassay intended for the direct and qualitative detection of SARS-CoV-2 nucleocapsid antigens in nasal swabs.)

Alternatively, for some applications, test 802 comprises a rapid molecular test kit, for example, an isothermal nucleic acid amplification (iNAAT) test, such as a rapid molecular test kit that uses a real-time loop mediated amplification reaction, such as the Lucira COVID-19 All-In-One Test Kit (Lucira Health, Inc., Emeryville, Calif., USA), or a nicking enzyme amplification reaction (NEAR) technology, such as the ID NOW™ (Abbott Laboratories, Abbott Park, Ill., USA), or a molecular test kit manufactured by Visby. Further alternatively, test 802 may comprise a CRISPR-based diagnostic test, an ELISA diagnostic test, or a spectroscopy-based diagnostic test.

Further alternatively, for some applications, test 802 comprises a lateral flow test strip other than a lateral flow immunoassay test strip. For example, test 802 may comprise:
a molecular test utilizing PCR in combination with a lateral flow test strip, such as for the qualitative, visual detection of nucleic acid from a virus, such as SARS-CoV-2; for example, the molecular test may be the Accula™ SARS-CoV-2 Test (Mesa Biotech, San Diego, Calif., USA); or
a CRISPR-based lateral flow test strip, such as a CRISPR/Cas9 lateral flow test strip, e.g., as described by Osborn M J et al. in “CRISPR/Cas9-Based Lateral Flow and Fluorescence Diagnostics,” Bioengineering 2021 (February), 8, 23, and/or by Xiong E et al. in “Simultaneous Dual-Gene Diagnosis of SARS-CoV-2 Based on CRISPR/Cas9-Mediated Lateral Flow Assay,” Angew. Chem. 2021, 133, 5367-5375, both of which articles are incorporated herein by reference.

Optionally, for some applications, test 802 may further comprise one or more filters configured to concentrate specimen sample 24 by filtration prior to testing for the presence of the particulate.

Nasal swab 822 comprises a swab tip 838 that comprises an absorbent material 844 for collecting the specimen sample. As used in the present application, including in the claims and Inventive Concepts, an “absorbent material” is a material that collects and retains a specimen sample, such as because of the physical or chemical structure and/or properties of the material (such as, for example, adhesive properties), and/or the structure formed by the material. For example, the fibers of synthetic flocking are arranged to be absorbent, even if the fibers in isolation from one another would not be absorbent. For example, swab tip 838 may be a flocked swab tip (e.g., comprising nylon), a rayon swab tip, a cotton swab tip, a polyurethane foam swab tip, a polyethylene terephthalate (PET) swab tip, a polyester swab tip, a foam swab tip (e.g., comprising polyurethane) (e.g., an open-cell foam swab), a sponge swab tip (e.g., comprising cellulose), a brush swab tip (e.g., similar to a cytology brush), and a nanofiber swab tip.

In some applications, the specimen samples collected using the collection techniques described herein are tested for the presence of a particulate using one or more of the techniques described in the patent applications incorporated hereinbelow by reference, or a technique known in the art. The particulate may be a virus (e.g., an Influenza virus or SARS-CoV-2), a bacterium (e.g., Streptococcus bacterium), any of the other particulates described hereinabove, or any of the other biological materials described hereinabove. For example, the particulate may be tested for the presence of a particulate by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby. Further alternatively, the particulate may be tested the presence of a particulate using a CRISPR-based diagnostic test, an ELISA diagnostic test, or a spectroscopy-based diagnostic test.

Reference is now made to FIG. 3, which is a schematic illustration of a nasal wash fluid dispenser 20, in accordance with an application of the present invention.

Reference is also made to FIGS. 4A-D, which are schematic cross-sectional illustrations of a method of collecting a specimen sample 24 from a subject, for example one or more specimen samples 24, in accordance with an application of the present invention.

As shown in FIGS. 4A-B, nasal wash fluid 26 is intranasally dispensed into a nasal cavity 22 of the subject, via one or both nostrils 28. In order to intranasally dispense nasal wash fluid 26, a nasal dispenser is inserted into one or both nostrils 28. For example, the nasal dispenser may comprise a conventional nasal dispenser known in the art (also known as a nasal applicator), a conventional nasal dropper known in the art, or a conventional ampoule known in the art, all of which are inserted into one nostril at a time. Alternatively, the nasal dispenser may comprise one of the nasal dispensers described herein, such as nasal wash fluid dispenser 20 or the other nasal dispensers described hereinbelow, which may be inserted into one or both nostrils 28. FIG. 4A shows nasal wash fluid dispenser 20 inserted into nasal cavity 22 via one of nostrils 28.

As shown in FIG. 4C, nasal wash fluid 26 is intranasally dispensed such that nasal wash fluid 26 washes biological material (typically, but necessarily, including saliva, which contains inter alia mucus) into an oropharynx 30 from (a) nasal cavity 22, (b) a nasopharynx 32, or (c) nasal cavity 22 and nasopharynx 32. Typically, nasal wash fluid 26 loosens the biological material in nasal cavity 22, nasopharynx 32, and/or oropharynx 30.

As shown in FIG. 4D, the method further comprises, thereafter, collecting, via an anterior opening 34 of an oral cavity 36 of the subject, specimen sample 24 that contains at least a portion of the biological material washed into oropharynx 30 by nasal wash fluid 26. (Anterior opening 34 is the opening of the mouth between the lips, between outside oral cavity 36 and inside oral cavity 36.)

Specimen sample 24 is typically collected into a collection receptacle 40, as shown in FIG. 4D.

In some applications, collection receptacle 40 used to collect specimen sample 24 comprises a stabilizer (e.g., to help maintain enzyme activity), an inhibitor (e.g., to help prevent RNA degradation), and/or a purifier. Alternatively or additionally, in some applications, after collection, specimen sample 24 is transferred to a liquid container of a testing device, such as any of the testing devices described in the patent applications incorporated herein by reference below; the liquid container of the testing device comprises a stabilizer, an inhibitor, and/or a purifier.

This method comprehensively samples multiple respiratory sites of the upper respiratory tract to collect biological material in a specimen sample 24 and optionally can be performed by the subject (patient) alone without exposing others to contagion. Alternatively, the method can be performed entirely or partially by somebody other than the subject, for example a healthcare provider or a relative of the subject. The method is simpler to perform than nasopharyngeal and pharyngeal swabbing conventionally performed for sample specimen collection, which are heavily dependent on the skill of the healthcare provider and are believed to result in many false negatives because of poor techniques. In addition, conventional swabbing cannot be performed by most subjects on themselves.

In some applications, the method comprises collection of one or more specimen samples 24 containing biological material from one or more of the following locations: nasal cavity 22, nasopharynx 32, and oropharynx 30, and, in some applications (such as when the method further comprises gargling and/or swishing, as described hereinbelow), a posterior pharynx 42, a hypopharynx 44 and lower airways, and in some applications, oral cavity 36, thereby sampling the entire area where pathogens are found in the upper aerodigestive tract.

Reference is still made to FIGS. 4A-D. As used in the present application, including in the claims and Inventive Concepts, “biological material” includes, but is not limited to, microorganisms, antigens, human cells, extracellular soluble and non-soluble particles, cellular components and products, blood products, cellular biomarkers, hormones, vitamins, electrolytes, chemical mediators from cells such as mediators of inflammation, pollens, mucus, saliva, sputum, respiratory particles, droplets derived from the upper and lower airways, nucleic acids including DNA and RNA, and chemicals originating from external vapors. As used in the present application, including in the claims and Inventive Concepts, microorganisms may include either pathogenic microorganisms or non-pathogenic microorganisms or both, for example, viruses, bacteria, protozoa, and fungi.

As used in the present application, including in the claims and Inventive Concepts, human cells may include epithelial cells, for example, goblet cells and/or columnar epithelial cells primarily derived from nasal cavity 22 and/or squamous epithelial cells primarily derived from oral cavity 36. As used in the present application, including in the claims and Inventive Concepts, human cells may also include cellular responders of the immune system, for example, leukocytes (e.g., neutrophils, eosinophils, lymphocytes, or monocytes), macrophages, mast cells, and histocytes. As used in the present application, including in the claims and Inventive Concepts, cellular components and products include lipids, proteins, glycoproteins, proteoglycans, mucins, and nucleic acids such as DNA and RNA.

Reference is again made to FIG. 4C. For some applications, the subject tilts his or her head back before, while, and/or after nasal wash fluid 26 is intranasally dispensed into nasal cavity 22, e.g., such as described in more detail hereinbelow. Alternatively, the subject holds his or her head in a neutral position or tilted forward while nasal wash fluid 26 is intranasally dispensed into nasal cavity 22, and optionally tilts his or her head back after nasal wash fluid 26 is intranasally dispensed into nasal cavity 22. Although tilting of the subject's head back and forward is generally described herein as being performed by the subject himself or herself, the subject's head may alternatively be tilted by somebody else, such as if the subject is too young or otherwise unable to tilt his or her own head.

For any of the applications described herein, intranasally dispensing nasal wash fluid 26 into nasal cavity 22 may comprise intranasally dispensing nasal wash fluid 26 into nasal cavity 22 while the subject is lying supine. For example, this position may be particularly appropriate when a conventional nasal dropper or a conventional ampoule is used for dispending nasal wash fluid 26.

In some applications, the method further comprises waiting for a period of time between (a) concluding intranasally dispensing nasal wash fluid 26 into nasal cavity 22 and (b) collecting specimen sample 24, the period of time at least 1 minute, e.g., at least 2 minutes, at least 3 minutes, or at least 5 minutes, such as at least 10 minutes. Optionally, the period of time is at least 15 minutes, or at least 30 minutes. The inventors found that waiting for a period of time less than 90 minutes between intranasally dispensing nasal wash fluid 26 into nasal cavity 22 and collecting specimen sample 24 increased the quantity of virus in the collected specimen sample. The inventors hypothesize that waiting for the period of time provides enough time for the subject's natural nasal mucociliary clearance (NMC) system to transport nasal wash fluid 26 containing biological material (typically including mucus) from nasal cavity 22 into oropharynx 30.

Reference is again made to FIG. 4B. In some applications, the method comprises dispensing intranasally a total volume of 0.2-20 mL of a nasal wash fluid 26, for example, 0.2-10 mL, for example, 0.2-5 mL, for example, 0.6-5 mL, into nasal cavity 22. The total nasal wash fluid 26 volume of 0.2-20 mL describes the total volume introduced into the entire nasal cavity 22 through either one or both nostrils 28. For example, 0.1-10 mL may be dispensed into each nostril 28, for a total volume of 0.2-20 mL, or for example, 0.2-20 mL may be dispensed into one nostril 28. Experiments performed by the inventors showed that fluid drained into oropharynx 30 when 1 mL of fluid was dispensed into nasal cavity 22, and did not drain into oropharynx 30 enough to be expressed out of the mouth when only 0.5 mL of fluid was dispensed into nasal cavity 22. However, the inventors hypothesize that dispensing a smaller volume of nasal wash fluid 26, such as little as 0.2 mL, into nasal cavity 22 may be adequate for draining into oropharynx 30 and expressing out of the mouth if nasal wash fluid 26 is dispensed at a higher pressure and/or is better aimed toward nasopharynx 32 than in the experiments performed.

In some applications, nasal wash fluid 26 is intranasally dispensed as one or more types of fluid discharge selected from the group consisting of: a fluid stream, a spray, a mist, a pressurized aerosol, steam, and an atomized fluid.

In some applications, nasal wash fluid 26 is dispensed intranasally using a circular rotary motion that generates dispersion within nasal cavity 22.

In some applications, nasal wash fluid 26 is intranasally dispensed in a plurality of pulses so as to increase volume without sacrificing comfort; for example, each of the pulses may have a volume of 0.02-10 mL, for example, 0.02-1 mL. Lowering the volume or the pressure of the fluid pulses reduces discomfort and unintentional swallowing, and a plurality of pulses increases volume and surface area sampled, thereby increasing the amount of total particulate collected in specimen sample 24, for improved sampling.

In some applications, nasal wash fluid 26 comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution.

Typically, nasal wash fluid 26 does not comprise a drug molecule. Alternatively, nasal wash fluid 26 does comprise a drug molecule.

For some applications, nasal wash fluid 26 comprises an odorant.

Reference is made to FIGS. 4B and 4C. In some applications, nasal wash fluid 26 is intranasally dispensed into nasal cavity 22 (either continuously or in a plurality of pulses) until the subject senses (e.g., by feeling and/or tasting) nasal wash fluid 26 in oropharynx 30. For applications which comprise gargling and/or swishing, as described herein, the subject typically gargles and/or swishes after sensing nasal wash fluid 26 in oropharynx 30.

For some of these applications, nasal wash fluid 26 comprises a tastant, for example, menthol flavoring, to monitor fluid passage and the movement of the biological material and nasal wash fluid 26 from nasal cavity 22 and nasopharynx 32 into oropharynx 30. Optionally, as mentioned above, the subject tilts his or her head back before, while, and/or after nasal wash fluid 26 is intranasally dispensed into nasal cavity 22, e.g., such as described in more detail hereinbelow. Further optionally, for applications in which nasal wash fluid 26 is separately intranasally dispensed into both nostrils, after the subject senses, in oropharynx 30, nasal wash fluid 26 administered to one of the nostrils, nasal wash fluid is intranasally dispensed into the other nostril, until the subject senses the additional nasal wash fluid in oropharynx 30.

As used in the present application, including in the claims and Inventive Concepts, a “tastant” is a taste-provoking chemical molecule that is dissolved in a fluid, and stimulates the sense of taste. For example, the tastant may comprise a non-salty flavor, such as menthol flavoring, a sweet flavor (e.g., comprising a sugar), a bitter flavor; a salty flavor, e.g., saline solution; and/or a non-bitter flavor.

Alternatively or additionally, in some applications, nasal wash fluid 26 comprises a color additive to monitor fluid passage and the movement of the biological material and nasal wash fluid 26 from nasal cavity 22 and nasopharynx 32 into oropharynx 30, and collecting specimen sample 24 comprises confirming that specimen sample 24 contains at least a portion of nasal wash fluid 26 by visually ascertaining that specimen sample 24 comprises the color additive. For some applications, visually ascertaining is performed by the human eye. Alternatively, for some applications, visually ascertaining is performed using spectroscopy, image processing, and/or three-dimensional (3D) image sensing; any of these techniques may optionally utilize artificial intelligence. Such confirmation may identify an inadvertent and/or a deliberate lack of administration of the nasal wash and/or passage of the nasal wash into oropharynx 30.

Alternatively or additionally, in some applications, nasal wash fluid 26 comprises a chemical additive (which may or may not have a color) to monitor fluid passage and the movement of the biological material and nasal wash fluid 26 from nasal cavity 22 and nasopharynx 32 into oropharynx 30, and collecting specimen sample 24 comprises confirming that specimen sample 24 contains at least a portion of nasal wash fluid 26 by ascertaining that specimen sample 24 includes the chemical additive. For some applications, ascertaining is performed using spectroscopy, image processing, and/or three-dimensional (3D) image sensing; any of these techniques may optionally utilize artificial intelligence. For example, ascertaining may be performed using an immunoassay (for example, a lateral-flow immunochromatographic assay, a chromatographic digital immunoassay, or an enzyme-linked immunosorbent assay (ELISA)); or a molecular-based assay (for example, nucleic acid hybridization, or for example, nucleic acid amplification, including polymerase chain reaction (PCR) amplification, real-time quantitative PCR (qPCR) amplification, reverse transcriptase PCR (RT-PCR) amplification, and isothermal amplification, such as loop-mediated isothermal amplification (LAMP)). Such confirmation may identify an inadvertent and/or a deliberate lack of administration of the nasal wash and/or passage of the nasal wash into oropharynx 30.

Further alternatively or additionally, in some applications, nasal wash fluid 26 comprises a chemical that increases the amount of biological material in specimen sample 24 by enhancing the clearance of the mucus as well as dislodging the biological material lining the mucosa, for example, a mucolytic agent such as acetylcysteine, or for example, a detergent such as polysorbate, or for example, a gas such as pressurized carbon dioxide to generate bubbles or nitrous oxide to increases the speed of mucociliary clearance.

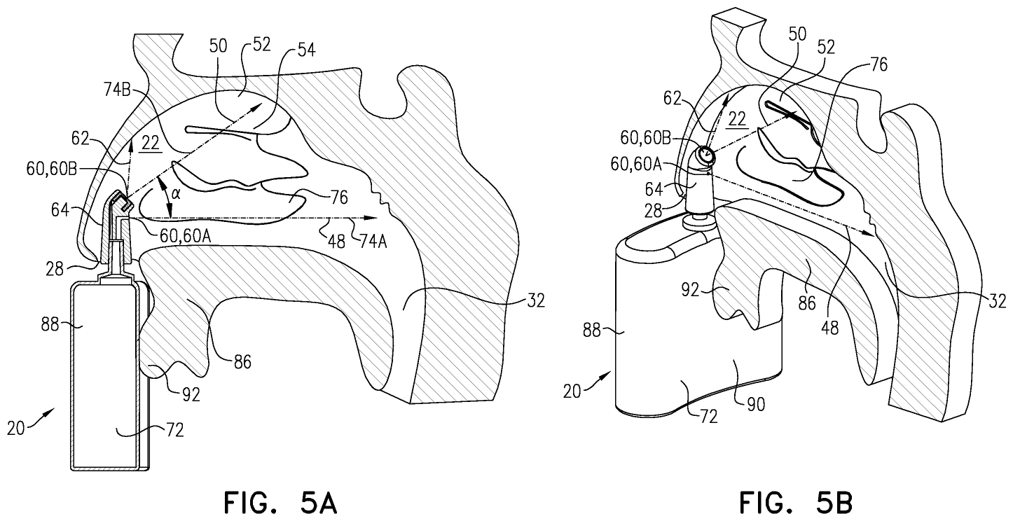

Reference is made to FIGS. 5A-B, which are schematic cross-sectional illustrations of nasal wash fluid dispenser 20 inserted into nasal cavity 22, in accordance with an application of the present invention. FIG. 5B shows nasal wash fluid dispenser 20 rotated slightly along a lateral axis compared to FIG. 5A, so as to provide an additional perspective.

In some applications, the method described hereinabove with reference to FIGS. 4A-D comprises dispensing nasal wash fluid 26 multidirectionally into each of the one or two fossae of nasal cavity 22. In some applications, intranasally dispensing nasal wash fluid 26 multidirectionally within nasal cavity 22 comprises dispensing nasal wash fluid 26 into nasal cavity 22 in a posterior direction 48 toward nasopharynx 32 and in a superoposterior direction 50 toward an olfactory area 52 of nasal cavity 22 above a superior nasal concha 54, for example, through a plurality of nozzles 60, for example, through exactly two nozzles 60.

As used in the present application, including in the claims and Inventive Concepts, a “nozzle” is a device designed to control the direction or characteristics of a fluid flow as it exits an enclosed chamber or tube.

In some applications, intranasally dispensing nasal wash fluid 26 multidirectionally within nasal cavity 22 comprises intranasally dispensing nasal wash fluid 26 within nasal cavity 22 via the plurality of nozzles 60 with respective different fluid flow characteristics, the fluid flow characteristics including one or more characteristics selected from the group consisting of: volume, sprayed particle size, velocity, and flow rate. For example, in some applications, the volume of nasal wash fluid 26 dispensed in posterior direction 48 may be greater than the volume of nasal wash fluid 26 dispensed in superoposterior direction 50 so as to increase the amount of biological material collected in specimen sample 24, since the majority of the biological material contained within nasal cavity 22 drains towards nasopharynx 32 and therefore is believed to be a prime site for sampling. In other applications, for example, the volume of nasal wash fluid 26 dispensed in superoposterior direction 50 may be greater than the volume of nasal wash fluid 26 dispensed in posterior direction 48. These applications may be beneficial since the inventors hypothesize that some viruses may be more concentrated in olfactory area 52, and that reflexive swallowing or premature spitting of nasal wash fluid 26 by the subject may sometimes be induced if a large volume of fluid is dispensed posteriorly.

In some applications, intranasally dispensing nasal wash fluid 26 multidirectionally within nasal cavity 22 comprises intranasally dispensing nasal wash fluid 26 within nasal cavity 22 via the plurality of nozzles 60 with equal fluid flow characteristics, the fluid flow characteristics including one or more characteristics selected from the group consisting of: volume, sprayed particle size, velocity, and flow rate.

In some applications, intranasally dispensing nasal wash fluid 26 multidirectionally within nasal cavity 22 comprises intranasally dispensing nasal wash fluid 26 within nasal cavity 22 via the plurality of nozzles 60 either simultaneously or sequentially.

In some applications, the method described hereinabove with reference to FIGS. 4A-D comprises dispensing nasal wash fluid 26 unidirectionally into both nostrils 28 (and thus into both fossae of the nasal cavity), for example, through a single nozzle 60. In some applications, intranasally dispensing nasal wash fluid 26 unidirectionally within nasal cavity 22 comprises dispensing nasal wash fluid 26 into nasal cavity 22 in posterior direction 48 toward nasopharynx 32 near a pharyngeal recess 58, or in superoposterior direction 50 toward olfactory area 52, or in a superior direction 62. For these applications, nasal wash fluid dispenser 20 may comprise only a single nozzle 60 (configuration not shown), or a conventional nasal dispenser known in the art may be used.

Dispensing nasal wash fluid 26 to target the posterior aspect of nasal cavity 22 toward nasopharynx 32 may increase the quality of the collected specimen sample 24 because a majority of the biological material contained within nasal cavity 22 drains toward nasopharynx 32, which is therefore a prime site for sampling. Alternatively or additionally, dispensing nasal wash fluid 26 to target the superoposterior aspect of nasal cavity 22 toward olfactory area 52 may increase the quality of the collected specimen sample 24 because olfactory area 52 contains high levels of infectious particles in infected individuals, such as with viruses, for example, SARS-CoV-2, in infected individuals with or without symptoms of olfactory dysfunction. A case study analyzed MRIs of a patient with confirmed COVID-19 who had experienced symptomatic olfactory dysfunction and determined that there were no anomalies present in the olfactory bulb and track, but that there was significant bilateral inflammation of the olfactory cleft in the nasal cavity. (Eliezer M, Hautefort C, Hamel A, et al. (2020). “Sudden and Complete Olfactory Loss Function as a Possible Symptom of COVID-19.” JAMA Otolaryngol Head Neck Surg. Published online Apr. 8, 2020. doi:10.1001/jamaoto.2020.0832). This case study supports the hypothesis that olfactory area 52 around the olfactory cleft above superior nasal concha 54 in nasal cavity 22 may contain high levels of infectious particles in individuals infected with SARS-CoV-2.

In some applications, intranasally dispensing nasal wash fluid into nasal cavity 22 comprises inserting a tubular applicator 64 of nasal wash fluid dispenser 20 into a nostril 28 of the subject, for example, inserting two tubular applicators 64 of nasal wash fluid dispenser 20 into both nostrils 28 of the subject.

Reference is again made to FIGS. 4B-C. In some applications, after nasal wash fluid 26 is dispensed into nasal cavity 22, the subject then sniffs or snorts, one or more times, the intranasally-introduced nasal wash fluid 26 posteriorly further into nasal cavity 22, allowing nasal wash fluid 26 to reach nasopharynx 32, from where nasal wash fluid 26 then drains into oropharynx 30. In other words, the sniffing or snorting moves nasal wash fluid 26 within nasal cavity 22 posteriorly toward nasopharynx 32 from where it subsequently drains to oropharynx 30. The sniffing or snorting comprises swiftly drawing air through the nose in short, usually audible, inhalations, that are more sudden and stronger than ordinary breathing. The sniffing or snorting does not necessarily comprise smelling. Alternatively, the intranasally-introduced nasal wash fluid 26 moves posteriorly further into nasal cavity 22 to reach nasopharynx 32 through normal mucociliary movement, without any special action by the subject, such as by sniffing or snorting. Nasal wash fluid 26 then drains from nasopharynx 32 into oropharynx 30, such as shown in FIG. 4C. In other words, the normal mucociliary movement moves nasal wash fluid 26 within nasal cavity 22 posteriorly toward nasopharynx 32, from where nasal wash fluid 26 subsequently drains to oropharynx 30.

In some applications, collecting specimen sample 24 from oral cavity 36 of the subject is performed by the subject alone, while in other applications, collecting specimen sample 24 from oral cavity 36 of the subject is performed by someone other than the subject, for example, by a healthcare provider or relative of the subject.

Reference is again made to FIG. 4D. In some applications, collecting specimen sample 24 comprises collecting specimen sample 24 from oral cavity 36 of the subject by the subject expressing, e.g., spitting, out the contents of oral cavity 36 from oral cavity 36 via anterior opening 34. Typically, the contents contain at least a portion of the intranasally-introduced nasal wash fluid 26 and at least a portion of the biological material. For example, the subject may spit out at least a portion of the intranasally-introduced nasal wash fluid 26 and at least a portion of an orally-introduced oral wash fluid 68 from oral cavity 36. In some applications, collecting the one or more specimen samples 24 comprises collecting a specimen sample 24 that was spit, by the subject, out of oral cavity 36 via anterior opening 34, which typically contains at least a portion of the intranasally-introduced nasal wash fluid 26 and at least a portion of the biological material.

Typically, in applications in which specimen sample 24 is collected by the subject expressing, e.g., spitting, out the contents of oral cavity 36, specimen sample 24 is collected into collection receptacle 40, such as by the subject directly expressing, e.g., spitting, specimen sample 24 into collection receptacle 40. Collection receptacle 40 may, for example, comprise a cup, as shown, or may alternatively comprise a collection receptacle of a sample processing device or sampling testing device, such as described, for example, in one or more of the patent applications incorporated hereinbelow by reference, or otherwise known in the art.

For any of the applications described herein that comprise spitting by the subject, the spitting optionally comprises hawking, i.e., forcefully spitting after forcing material up from the throat, e.g., by making a harsh guttural sound.

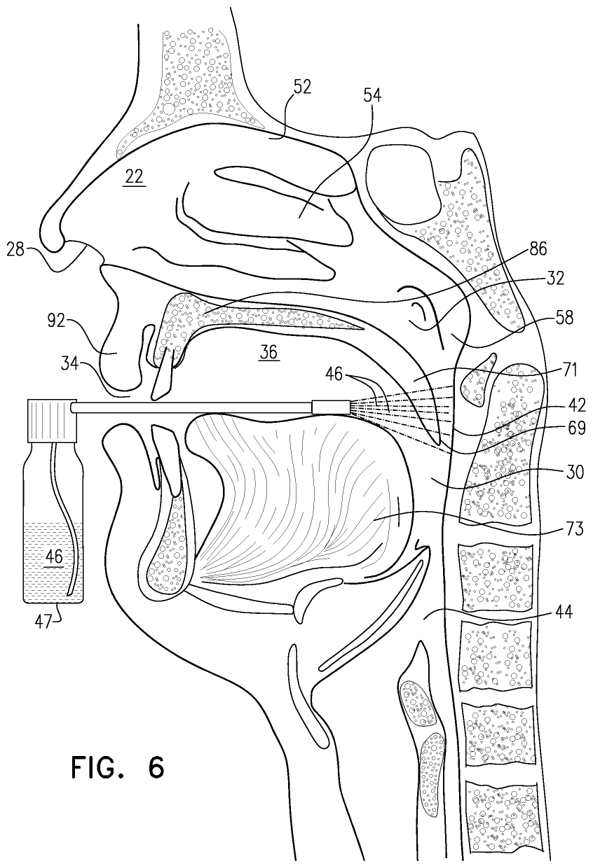

Reference is now made to FIG. 6, which is a schematic cross-sectional illustration of a portion of a method for collecting specimen sample 24 from the subject, in accordance with an application of the present invention. In some applications, the method of FIGS. 4A-D further comprises, before collecting specimen sample 24, spraying, via anterior opening 34 of oral cavity 36, oropharynx 30 with oropharyngeal fluid wash 46. This spraying may help loosen and increase the collection of biological material from the walls of oropharynx 30 (e.g., the walls of posterior pharynx 42), and/or may provide better coverage of posterior pharynx 42 than gargling and/or swishing alone, and thus result in better sampling of posterior pharynx 42 than gargling and/or swishing alone. This spraying is typically performed after intranasally dispensing nasal wash fluid 26 into nasal cavity 22, but may alternatively be performed prior to intranasally dispensing nasal wash fluid 26 into nasal cavity 22.

Reference is again made to FIGS. 4A-D. In some applications, the nasally-introduced nasal wash fluid 26, after it has been washed into oropharynx 30, such as shown in FIG. 4C, is gargled by the subject, swished by the subject in oral cavity 36, or both gargled and swished by the subject in oral cavity 36, prior to collecting specimen sample 24 from oral cavity 36, such as shown in FIG. 4D. In some of these applications, the subject alternates multiple times between (a) gargling and/or swishing and (b) sniffing or snorting, before collecting specimen sample 24 from oral cavity 36. Typically, the alternation between (a) gargling and/or swishing and (b) sniffing or snorting occurs while nasal wash fluid 26 remains in oropharynx 30 of the subject. In general, the subject tilts his or her head back during all of the gargling and/or swishing steps of the methods described and claimed herein. As used in the present application, including in the claims and Inventive Concepts, “gargling” means holding a liquid in the throat (typically in the oropharynx) and agitating with air expelled from the lungs. In general, during gargling, the subject avoids swallowing to the extent reasonably possible.

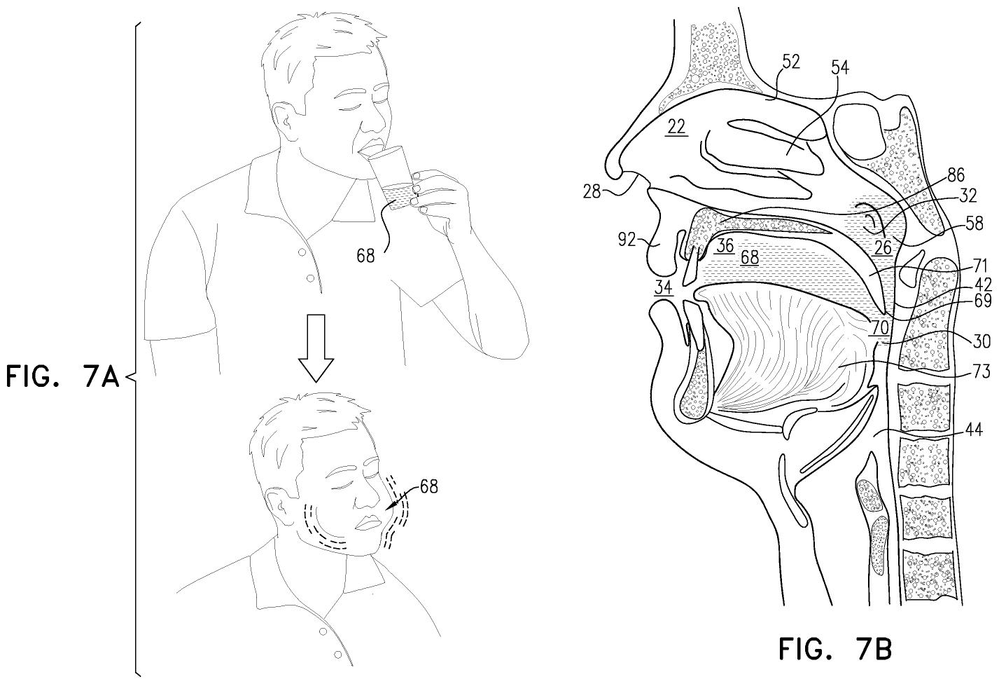

Reference is still made to FIGS. 4A-D and is additionally made to FIGS. 7A-B, which are schematic illustrations of a portion of a method for collecting specimen sample 24 from the subject, in accordance with an application of the present invention. In some applications, the method of FIGS. 4A-D further comprises, before collecting specimen sample 24, orally dispensing oral wash fluid 68 into oral cavity 36 via anterior opening 34 (typically by the subject), such as shown in FIG. 7A, before or after intranasally introducing nasal wash fluid 26, to combine with the intranasally-introduced nasal wash fluid 26 to form a mixture 70 in oropharynx 30, such as shown in FIG. 7B. Thereafter, the subject gargles mixture 70, and specimen sample 24 is collected from oral cavity 36. Specimen sample 24 contains at least a portion of mixture 70 and the at least a portion of the biological material washed into oropharynx 30 with nasal wash fluid 26. Optionally, the subject additionally swishes mixture 70 in oral cavity 36.

In some applications, the method of FIGS. 4A-D further comprises, before collecting specimen sample 24, orally dispensing oral wash fluid 68 into oral cavity 36 via anterior opening 34 (typically by the subject), such as shown in FIG. 7A, before or after intranasally introducing nasal wash fluid 26. The subject gargles oral wash fluid 68 such that oral wash fluid 68 forms mixture 70 with the intranasally-introduced nasal wash fluid 26 in oropharynx 30 and/or oral cavity 36, such as shown in FIG. 7B. Specimen sample 24 contains at least a portion of mixture 70 and the at least a portion of the biological material washed into oropharynx 30 with nasal wash fluid 26. Optionally, the subject additionally swishes mixture 70 in oral cavity 36.

In some applications, the method of FIGS. 4A-D further comprises, before collecting specimen sample 24, orally dispensing oral wash fluid 68 into oral cavity 36 via anterior opening 34 (typically by the subject), such as shown in FIG. 7A, before or after intranasally introducing nasal wash fluid 26. The subject gargles oral wash fluid 68. Specimen sample 24 contains at least a portion of oral wash fluid 68, the at least a portion of the biological material washed into the oropharynx with the nasal wash fluid, and typically, but not necessarily, a portion of nasal wash fluid 26. Optionally, the subject additionally swishes oral wash fluid 68 in oral cavity 36.

In some applications, the method of FIGS. 4A-D further comprises, before collecting specimen sample 24, orally dispensing oral wash fluid 68 into oral cavity 36 via anterior opening 34 (typically by the subject), such as shown in FIG. 7A, before or after intranasally introducing nasal wash fluid 26. The subject swishes oral wash fluid 68 in oral cavity 36. Specimen sample 24 contains at least a portion of oral wash fluid 68, the at least a portion of the biological material washed into the oropharynx with the nasal wash fluid, and typically, but not necessarily, a portion of nasal wash fluid 26.

In some applications, collecting specimen sample 24 comprises collecting specimen sample 24 from oral cavity 36 of the subject by the subject expressing, e.g., spitting, out the contents of oral cavity 36 from oral cavity 36 via anterior opening 34. For example, the subject may spit out at least a portion of the intranasally-introduced nasal wash fluid 26 and at least a portion of orally-introduced oral wash fluid 68 from oral cavity 36.

Experiment 8, described in the above-mentioned U.S. application Ser. No. 17/921,672 and International Application PCT/IL2021/050519, provides evidence of the efficacy of these techniques for collecting specimen samples for detection of SARS-CoV-2 using quantitative PCR (qPCR).

For example, 5-10 mL of oral wash fluid 68 may be orally dispensed into oral cavity 36 via anterior opening 34.

In some applications, the subject alternates multiple times between gargling and sniffing or snorting, before collecting specimen sample 24 from oral cavity 36. Typically, the alternation between gargling and sniffing or snorting occurs while nasal wash fluid 26 and/or oral wash fluid 68 remains in oropharynx 30 of the subject.

For any of the applications described herein, orally dispensing oral wash fluid 68 into oral cavity 36 via anterior opening 34 of oral cavity 36 may comprise orally dispensing oral wash fluid 68 into oral cavity 36 via anterior opening 34 of oral cavity 36 while the subject is sitting or standing in a vertical position.

In some applications, oral wash fluid 68 comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution.

Typically, oral wash fluid 68 does not comprise a drug molecule. Alternatively, oral wash fluid 68 comprises a drug molecule.

In some applications, oral wash fluid 68 comprises a tastant, for example, menthol flavoring. In some applications, nasal wash fluid 26 and oral wash fluid 68 contain different chemical components that, when combined, generate an indicator. For example, the indicator may be bubbling that is produced by a chemical reaction generated by combining the different chemical components of nasal wash fluid 26 and oral wash fluid 68.

In some applications, nasal wash fluid 26 comprises a first color additive, and oral wash fluid 68 comprises a second colored additive, and when combined, a third color is generated, for example, by mixing of the two color additives, or for example, by a chemical reaction. In some applications, collecting specimen sample 24 comprises confirming that specimen sample 24 contains at least a portion of nasal wash fluid 26 and at least a portion of oral wash fluid 68 by visually ascertaining that specimen sample 24 has an indicator produced by a combination of nasal wash fluid 26 and oral wash fluid 68, for example, a third color produced by a combination of the first and the second color additives. For some applications, visually ascertaining is performed by the human eye. Alternatively, for some applications, visually ascertaining is performed using spectroscopy, image processing, and/or three-dimensional (3D) image sensing; any of these techniques may optionally utilize artificial intelligence.

Alternatively or additionally, in some applications, oral wash fluid 68 comprises a chemical that will increase the amount of biological material in specimen sample 24 by enhancing the clearance of the mucus as well as dislodging the biological material lining the mucosa, for example, a mucolytic agent such as acetylcysteine, or for example, a detergent such as polysorbate, or for example, a gas such as pressurized carbon dioxide to generate bubbles.

Alternatively, in some applications, collecting specimen sample 24 comprises collecting a specimen sample from oral cavity 36 of the subject by aspirating out (using suction) at least a portion of nasal wash fluid 26 and at least a portion of the biological material from oral cavity 36 via anterior opening 34, for example, using a catheter. For example, the at least a portion of the intranasally-introduced nasal wash fluid and orally introduced oral wash fluid 68 may be aspirated out from oral cavity 36 after gargling by the subject. Alternatively or additionally, in some applications, collecting a specimen sample comprises collecting a specimen sample from oral cavity 36 of the subject by drawing out at least a portion of nasal wash fluid 26 and at least a portion of the biological material from oral cavity 36, using absorbent material. For example, the at least a portion of the intranasally-introduced nasal wash fluid and orally introduced oral wash fluid 68 may be drawn from oral cavity 36 using absorbent material after gargling by the subject.

Gargling nasal wash fluid 26 or mixture 70 of nasal wash fluid 26 and oral wash fluid 68 predominately bathes the anterior oropharynx, including the tonsils, tonsillar pillars, uvula 69, soft palate 71, and posterior tongue 73. In this application, the method thus allows at least a portion of the intranasally-introduced nasal wash fluid that sampled nasal cavity 22 and nasopharynx 32 to also sample oropharynx 30 via gargling and to then be collected from oral cavity 36. Additionally, gargling may also allow for collection of respiratory particles and droplets expressed from or found on posterior pharynx 42, hypopharynx 44, oral cavity 36, and lower airways since the act of gargling is a process of exhaling. Therefore, specimen sample 24 collected by this method may contain droplets and particles from the entire respiratory tract including both upper and lower airways. Typically, the collected specimen sample contains at least portion of the nasally-introduced nasal wash fluid solution and at least a portion of the orally introduced oral wash fluid 68 solution and at least a portion of the biological material that originated from the upper aerodigestive tract. Alternatively, the collected specimen sample contains at least portion of the nasally-introduced nasal wash fluid solution and at least a portion of the biological material that originated from the upper aerodigestive tract.

Typically, human cells collected in specimen sample 24 may include epithelial cells, for example, columnar epithelial cells primarily derived from the nasal cavity and squamous epithelial cells primarily derived from the oral cavity. Typically, human cells collected in specimen sample 24 may also include cellular responders of the immune system, for example, neutrophils, eosinophils, lymphocytes, monocytes, macrophages, mast cells, and histocytes.

For some applications, the method further comprises waiting for a period of time between (a) concluding intranasally dispensing nasal wash fluid 26 into nasal cavity 22 and (b) orally dispensing oral wash fluid 68 into oral cavity 36, the period of time at least 1 minute, e.g., at least 2 minutes, at least 3 minutes, or at least 5 minutes, such as at least 10 minutes. Optionally, the period of time is at least 15 minutes, or at least 30 minutes. The inventors found that waiting for a period of time less than 90 minutes between intranasally dispensing nasal wash fluid 26 into nasal cavity 22 and orally dispensing oral wash fluid 68 into oral cavity 36 increased the quantity of virus in the collected sample.

For some applications, specimen sample 24 is a first specimen sample, and mixture 70 is a first mixture, and the method further comprises, after collecting the first specimen sample, waiting for a period of time, the period of time at least 1 minute, e.g., at least 2 minutes, at least 3 minutes, or at least 5 minutes, such as at least 10 minutes. Optionally, the period of time is at least 15 minutes, or at least 30 minutes. After waiting, additional oral wash fluid 68 is orally dispensed into oral cavity 36 via anterior opening 34 of oral cavity 36 such that the additional oral wash fluid 68 forms a second mixture with nasal wash fluid 26 in oropharynx 30. Thereafter, the subject gargles the second mixture. Thereafter, a second specimen sample is collected that passed out of anterior opening 34 of oral cavity 36 and contains at least a portion of the second mixture and the at least a portion of the biological material washed into oropharynx 30 with nasal wash fluid 26. The method described in this paragraph typically does not comprise intranasally dispensing nasal wash fluid into nasal cavity 22 after collecting the first specimen sample. Optionally, the period of time is at least 15 minutes, or at least 30 minutes. The inventors found that virus was detected in both the first and second specimen samples, even though additional nasal wash was not intranasally dispensed before the additional oral wash fluid 68 was dispensed and gargled.

Reference is again made to FIG. 4D. In some applications, collecting the one or more specimen samples 24 comprises collecting one or more fluid specimen samples 24. In some applications, collecting one or more specimen samples 24 consists collecting at least two specimen samples, for example, collecting exactly two specimen samples. In some applications, collecting the at least two specimen samples comprises collecting further specimen samples in addition to collecting specimen sample 24 from oral cavity 36, for example, collecting an additional specimen sample by swabbing nasal cavity 22 or by collecting an additional specimen sample by inserting absorbent material into nasal cavity 22, typically after collecting fluid specimen sample 24 from oral cavity 36. In some applications, first specimen sample 24 contains at least a first portion of nasal wash fluid 26 and at least a first portion of the biological material, for example collected from oral cavity 36, and a second specimen sample contains at least a second portion of the portion of nasal wash fluid 26 and at least a second portion of the biological material, for example collected from nasal cavity 22.

In some applications, the two or more specimen samples are combined to produce a combined specimen sample.

In some applications, collecting the second specimen sample comprises collecting from nasal cavity 22 by the subject blowing specimen sample 24 out of nasal cavity 22 via the one or two nostrils 28. In some applications, collecting the second specimen sample comprises collecting the second specimen sample that was blown, by the subject, out of nasal cavity 22 via the one or two nostrils 28.

In other applications, collecting the second specimen sample comprises collecting from nasal cavity 22 by aspirating specimen sample 24 out of nasal cavity 22 via the one or two nostrils 28, for example, using a catheter.

In some applications, collecting the second specimen sample by aspirating specimen sample 24 out from nasal cavity 22 via the one or two nostrils 28 comprises using a negative pressure vacuum generated by nasal wash fluid dispenser 20 after releasing nasal wash fluid 26, for example, by using nasal wash fluid dispenser 20 in accordance with an application of the present invention.

In some applications, collecting a second specimen sample from nasal cavity 22 comprises, after collecting a specimen sample from oral cavity 36 in collection receptacle 40, collecting residual biological material and nasal wash fluid 26 from nasal cavity 22 by blowing out of the nose of the subject into a plastic bag that is attached to collection receptacle 40. For example, the plastic bag may have a distal opening that is hermetically sealed to the proximal opening of collection receptacle 40 and may have a proximal opening that has a sealable closure mechanism, to allow for specimen sample 24 blown out of the nose of the subject into the plastic bag to join with specimen sample 24 collected from oral cavity 36 in collection receptacle 40. There may be a sealable opening or spout in collection receptacle 40 or in the plastic bag that is openable to allow exit of the combined specimen sample for testing.

Reference is again made to FIG. 4B. In some applications, instead of or in addition to intranasally dispensing nasal wash fluid 26 into nasal cavity 22, a vapor (e.g., a warm vapor) is intranasally dispensed into nasal cavity 22 to loosen the biological material in nasal cavity 22. Typically, intranasally dispensing the vapor comprises inhaling, by the subject, the vapor into nasal cavity 22, and, typically, into oropharynx 30 via nasopharynx 32, such that the vapor washes the biological material into oropharynx 30 from (a) nasal cavity 22, (b) nasopharynx 32, or (c) nasal cavity 22 and nasopharynx 32. Typically, specimen sample 24 contains at least a portion of the biological material washed into oropharynx 30 by the vapor.

Reference is made to FIGS. 4A-7B. The sampling methods described herein with reference to FIGS. 4A-7B typically do not comprise swabbing any portion of the subject, unless the methods are specifically described as comprising swabbing a portion of the subject. Alternatively, the sampling methods described herein may optionally comprise swabbing a portion of the subject.

The sampling methods described herein with reference to FIGS. 4A-7B typically do not comprise swabbing nasal cavity 22, unless the methods are specifically described as comprising swabbing nasal cavity 22. Alternatively, the sampling methods described herein may optionally comprise swabbing nasal cavity 22.

The sampling methods described herein with reference to FIGS. 4A-7B typically do not comprise swabbing nasopharynx 32, unless the methods are specifically described as comprising swabbing nasopharynx 32. Alternatively, the sampling methods described herein may optionally further comprise swabbing nasopharynx 32.

In some applications of the present invention, the methods with reference to FIGS. 4A-7B do not comprise swabbing nasopharynx 32, and specimen sample 24 is collected by swabbing oropharynx 30. For some of these applications, the methods do not comprise swabbing any portion of nasal cavity 22. Alternatively, for some of these applications, collecting specimen sample 24 comprises swabbing oropharynx 30 and swabbing one or both anterior nares of nasal cavity 22. Alternatively or additionally, for some of these applications, collecting specimen sample 24 comprises swabbing oropharynx 30 and swabbing one or more turbinates of nasal cavity 22 selected from the group consisting of: a middle turbinate and an inferior turbinate. As is known in the art, the anterior naris is the portion of the nasal cavity anterior to the internal nasal valve.

The sampling methods described herein with reference to FIGS. 4A-7B typically do not comprise swabbing oropharynx 30 and do not comprise swabbing nasopharynx 32, unless the methods are specifically described as comprising swabbing oropharynx 30 and/or nasopharynx 32. Alternatively, the sampling methods described herein may optionally further comprise swabbing oropharynx 30 and/or nasopharynx 32.

For some applications, collecting specimen sample 24 comprises drawing specimen sample 24 out of oral cavity 36 via anterior opening 34 of oral cavity 36 using an absorbent material (e.g., a flocked or cotton swab, or a sponge). (For example, an ORAcollect®⋅RNA Saliva Collection Device (DNA Genotek Inc., a subsidiary of OraSure Technologies, Inc. (Bethlehem, Pa., USA)) may be used.) Optionally, the absorbent material is located on a tip of a collector shaft, and specimen sample 24 is drawn out of oral cavity 36 via anterior opening 34 of oral cavity 36 using the absorbent material by inserting the tip of the collector shaft into oral cavity 36. For some of these applications, specimen sample 24 is drawn out of oral cavity 36 via anterior opening 34 of oral cavity 36 using the absorbent material by the subject sucking on the absorbent material.

For some applications in which the method does not comprise swabbing oropharynx 30 and does not comprise swabbing nasopharynx 32, specimen sample 24 is collected by drawing specimen sample 24 out of oral cavity 36 via anterior opening 34 of oral cavity 36 by contacting one or more portions of oral cavity 36 with an absorbent material (e.g., at a tip of a collector shaft), without swabbing oropharynx 30. (For example, the above-mentioned ORAcollect®⋅RNA Saliva Collection Device may be used.) For example, the one or more portions of oral cavity 36 may include one or more of buccal mucosa, the tongue (e.g., under the tongue), the gums (e.g., the lower gums), and/or the palatal mucosa. For example, for swabbing the lower gums, absorbent material (e.g., at a tip of a collector shaft) may be rubbed back and forth along the lower gums several times.

In applications of the present invention that comprise swabbing the subject, specimen sample 24 may be collected into collection receptacle 40 by placing the swab (or swabs) in collection receptacle 40.

In some applications, deriving the information from the extracellular vesicles comprises performing a pretesting step prior to deriving the information from the extracellular vesicles, comprising one or more of the following pretesting steps: chemical treatment, purification, filtration, enrichment, and/or centrifugation. In other applications, the information is derived from the extracellular vesicles present in specimen sample 24 without any pretesting steps.

Reference is again made to FIGS. 4A-D. In some applications of the present invention, a method is provided that comprises intranasally dispensing nasal wash fluid into nasal cavity 22 such that nasal wash fluid 26 passes from nasal cavity 22 into oropharynx 30 via nasopharynx 32. The method further comprises, thereafter, collecting a specimen sample that passed out of anterior opening 34 of oral cavity 36 and contains at least a portion of nasal wash fluid 26. In this method, biological material of interest for subsequent testing is not necessarily washed from nasal cavity 22 or nasopharynx 32 into oropharynx 30, although, of course, some biological material, such as mucus, will be thus washed. Instead, the biological material of interest for subsequent testing comes from elsewhere in the respiratory tract, typically at least oropharynx 30, and optionally lower in the respiratory tract. In this method, nasal wash fluid 26, upon arriving in oropharynx 30, may help loosen and increase the collection of biological material from the walls (e.g., of posterior pharynx 42) of oropharynx 30, and/or may provide better coverage of posterior pharynx 42 than gargling alone, and thus result in better sampling of posterior pharynx 42 than gargling alone.

This method may be practiced in combination with the other methods described herein, including, but not limited to gargling and/or spraying, via anterior opening 34 of oral cavity 36, oropharynx 30 with oropharyngeal fluid wash.

Reference is again made to FIG. 6. In some applications, a method is provided that comprises spraying (either by a healthcare worker or the subject), via anterior opening 34 of oral cavity 36, oropharynx 30 with oropharyngeal fluid wash 46. Thereafter, a specimen sample is collected that passed out of anterior opening 34 of oral cavity 36 (e.g., was spit out of anterior opening 34) and contains at least a portion of biological material washed off a pharyngeal wall (typically, the posterior pharyngeal wall) into oropharynx 30 of the subject by the oropharyngeal fluid wash. Specimen sample 24 may additionally or alternatively contain at least a portion of biological material washed off one or more of the following locations: posterior pharynx 42, a tonsillar area, soft palate 71, and posterior tongue 73. This method may be performed without intranasally dispensing nasal wash fluid into nasal cavity 22, or it may be performed in combination with such dispensing, such as described hereinabove. This method may be performed in combination with any of the techniques described herein. For some applications, a spray bottle 47 is provided that contains oropharyngeal fluid wash 46; spray bottle 47 may be an element of a kit comprising other elements described herein.

In some applications, the method further comprises, before collecting specimen sample 24, gargling the oropharyngeal fluid wash by the subject. This method may produce a better specimen sample than would be produced by gargling alone, because gargling alone generally results in the gargle fluid having limited contact with the posterior pharyngeal wall. Alternatively, the method does not comprise gargling the oropharyngeal fluid wash by the subject. Further alternatively or additionally, in some applications, the method further comprises, before collecting specimen sample 24, swishing, by the subject, the oropharyngeal fluid wash in oral cavity 36.

In some applications, the method further comprises, before collecting specimen sample 24, orally dispensing oral wash fluid 68 into oral cavity 36 via anterior opening 34 of oral cavity 36 such that oral wash fluid 68 forms a mixture with the oropharyngeal fluid wash in oropharynx 30; and gargling and/or swishing mixture 70 by the subject. In these applications, specimen sample 24 contains at least a portion of mixture 70 and the at least a portion of the biological material washed off the pharyngeal wall into oropharynx 30 by mixture 70.

In some applications, the spraying is performed while the subject says “ah” or “eh.” This may increase the amount of the oropharyngeal fluid wash that is deposited on the posterior pharyngeal wall, such as to build up enough volume to gargle and/or swish.

In some applications, a total volume of at least 2 mL (e.g. 2-15 mL, e.g. 2-10 mL) of the oropharyngeal fluid wash is sprayed.

In some applications, oropharynx 30 is sprayed with the oropharyngeal fluid wash in a plurality of pulses. For example, each of the pulses may have a volume of 0.05-5 mL.

In some applications, collecting specimen sample 24 comprises expressing (e.g., spitting), by the subject, specimen sample 24 out of oropharynx 30 via the anterior opening of oral cavity 36.

In some applications, such as labeled in FIG. 4A, a nasal wash fluid system 100 is provided that comprises nasal wash fluid dispenser 20, and further comprises nasal wash fluid 26, contained in dispensing container 72.

In some applications, nasal wash fluid 26 does not comprise a drug molecule. Alternatively, nasal wash fluid 26 does comprise a drug molecule.

In some applications, nasal wash fluid 26 comprises a non-irritant solution, which may, for example, comprise or consist of water. Alternatively, in some applications, nasal wash fluid 26 of a non-irritant solution consists of a saline solution, such as a phosphate-buffered saline solution.

In some applications, nasal wash fluid 26 comprises a tastant.

Alternatively or additionally, in some applications, nasal wash fluid 26 comprises a color additive. For example, the color additive may have a visually-perceptible color that is dominated by a non-white color other than yellow, such as red, green, or blue, which may be easier for a human subject to perceive in a liquid than yellow. Alternatively, the color additive may be yellow.

Further alternatively or additionally, in some applications, nasal wash fluid 26 comprises a chemical additive, such as described hereinabove with reference to FIGS. 4B and 4C.

In some applications, nasal wash fluid 26 comprises a first color additive, and nasal wash fluid system 100 further comprises an oral wash fluid 68 comprising a second colored additive, the first and the second color additives having different colors.

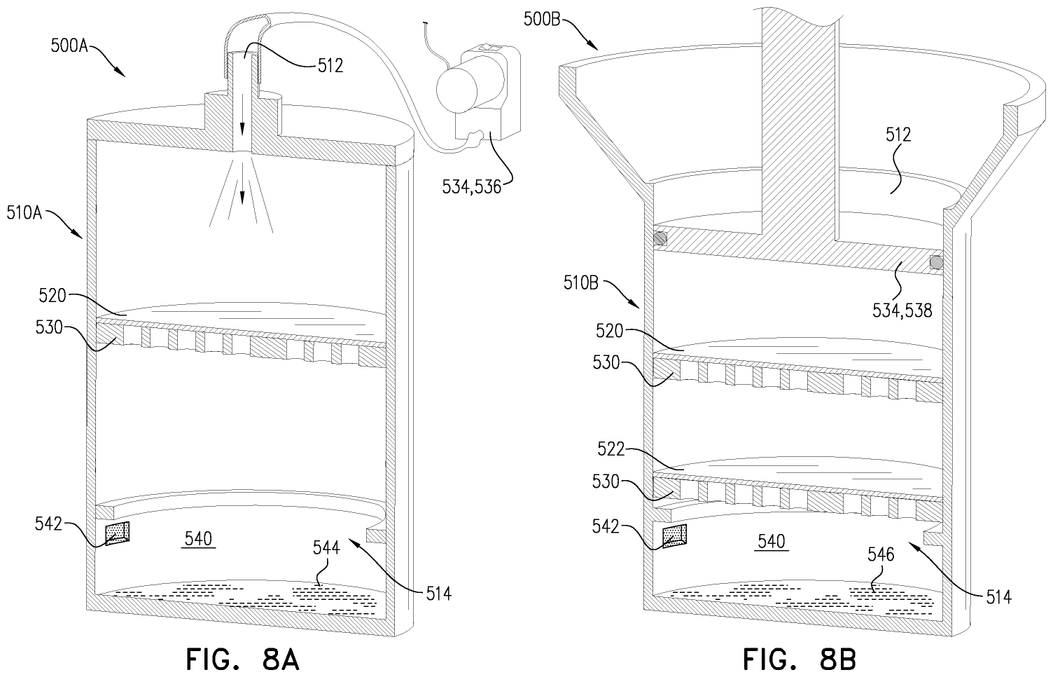

Reference is now made to FIGS. 8A-B, which are schematic illustrations of filter units 500A and 500B, respectively, for deriving information from extracellular vesicles present in saliva included in a liquid specimen sample, in accordance with respective applications of the present invention.

Filter unit 500A comprises at least one filter chamber 510A (such as exactly one, as shown), which is shaped so as to define an inlet 512, an outlet 514, and a fluid flow path between inlet 512 and outlet 514. Filter unit 500B comprises at least one filter chamber 510B (such as exactly one, as shown), which is shaped so as to define inlet 512, outlet 514, and a fluid flow path between inlet 512 and outlet 514.

Each of the at least one filter chambers 510A and 510B comprises a large-pored filter 520, which is disposed within at the at least one filter chamber in the fluid flow path, and has a nominal pore size.

The at least one filter chamber 510B of filter unit 500A comprises large-pored filter 520, and one or more additional filters 522, which are disposed within the at the at least one filter chamber 510B in series in the fluid flow path, and which have respective nominal pore sizes, which may or may not equal one another, and may or may not equal the nominal pore size of large-pored filter 520. For example, the one or more additional filters 522 may include exactly one additional filter 522 (as shown) or more than one additional filter 522 (configuration not shown). Typically, but not necessarily, each of the respective nominal pore sizes of the one or more additional filters 522 is less than or equal to the nominal pore size of large-pored filter 520.

For some applications, the nominal pore size of large-pored filter 520 is between 30 microns and 1.5 mm.

For some applications, the nominal pore size is representative of a minimum size of spherical particles (e.g., inert spherical particles) necessary for the respective filter to retain 85% of the spherical particles when distilled H₂O containing the spherical particles is passed through the respective filter at 20 degrees C. under pressure supplied by a 10 cm water column (of course, without downstream resistance) (herein, the “challenge-test nominal pore size”). It will be appreciated that the spherical particles, distilled H2O, and 10 cm water column are not elements of the device, but are instead non-device elements that are used to characterize certain physical properties of the filter(s), as is conventional for filter characterization in the filter art.

Alternatively, the nominal pore size may be characterized by a bubble point test, as is well known in the filter art, using a capillary flow porometer, such as CFP-1500A Automatic Capillary Flow Porometer (optionally, including options E, X, L, and/or M, if necessary for characterizing certain larger pore sizes) made by Porous Materials Inc. (PMI) (Ithaca, N.Y., USA), and a wetting liquid having a fluid surface tension of 15.9 dynes/cm, such as Galwick (PMI) (herein, the “bubble-point nominal pore size”).

As used in the present application, including in the claims and Inventive Concepts, a “pore” means any opening through a filter through which at least distilled H2O can pass.

For some applications, large-pored filter 520 comprises a depth filter.

For some applications, large-pored filter 520 comprises a mesh filter, such as a woven mesh filter or a non-woven mesh filter.

For some applications, large-pored filter 520 comprises fibers, such as crisscrossing fibers.

For some applications, large-pored filter 520 comprises a screen, such as a woven screen or a non-woven screen.

For some applications, large-pored filter 520 comprises a polymer, such as a polyester.

For some applications, large-pored filter 520 comprises a metal.

For some applications, the challenge-test nominal pore size is at least 40 microns. For some applications, the challenge-test nominal pore size is at least 50 microns. For some applications, the challenge-test nominal pore size is at least 60 microns. For some applications, the challenge-test nominal pore size is at least 100 microns. For some applications, the challenge-test nominal pore size is at least 120 microns. For some applications, the challenge-test nominal pore size is at least 150 microns. For some applications, the challenge-test nominal pore size is at least 200 microns. For some applications, the challenge-test nominal pore size is at least 500 microns.

For some applications, the challenge-test nominal pore size is less than 1 mm. For some applications, the challenge-test nominal pore size is less than 750 microns. For some applications, the challenge-test nominal pore size less than 500 microns. For some applications, the challenge-test nominal pore size less than 250 microns. For some applications, the challenge-test nominal pore size less than 200 microns.

For some applications, the challenge-test nominal pore size is between 30 and 40 microns. For some applications, the challenge-test nominal pore size is between 40 and 60 microns. For some applications, the challenge-test nominal pore size is between 50 and 200 microns, such as between 50 and 150 microns, e.g., between 100 and 150 microns. For some applications, the challenge-test nominal pore size is between 60 and 100 microns. For some applications, the challenge-test nominal pore size is between 100 and 120 microns. For some applications, the challenge-test nominal pore size is between 120 and 150 microns. For some applications, the challenge-test nominal pore size is between 150 and 200 microns. For some applications, the challenge-test nominal pore size is between 200 and 500 microns. For some applications, the challenge-test nominal pore size is between 500 microns and 1 mm, such as between 500 and 750 microns, or between 750 microns and 1 mm.

For some applications, the bubble-point nominal pore size is at least 40 microns. For some applications, the bubble-point nominal pore size is at least 50 microns. For some applications, the bubble-point nominal pore size is at least 60 microns. For some applications, the bubble-point nominal pore size is at least 100 microns. For some applications, the bubble-point nominal pore size is at least 120 microns. For some applications, the bubble-point nominal pore size is at least 150 microns. For some applications, the bubble-point nominal pore size is at least 200 microns.

For some applications, the bubble-point nominal pore size less than 500 microns. For some applications, the bubble-point nominal pore size less than 300 microns. For some applications, the bubble-point nominal pore size less than 250 microns. For some applications, the bubble-point nominal pore size less than 200 microns.

For some applications, the bubble-point nominal pore size is between 30 and 40 microns. For some applications, the bubble-point nominal pore size is between 40 and 60 microns. For some applications, the bubble-point nominal pore size is between 50 and 200 microns, such as between 50 and 150 microns, e.g., between 100 and 150 microns. For some applications, the bubble-point nominal pore size is between 60 and 100 microns. For some applications, the bubble-point nominal pore size is between 100 and 120 microns. For some applications, the bubble-point nominal pore size is between 120 and 150 microns. For some applications, the bubble-point nominal pore size is between 150 and 200 microns. For some applications, the bubble-point nominal pore size is between 200 and 300 microns.

For some applications, filter chambers 510A and 510B are generally cylindrical, as shown, while for other applications they may have different shapes.

For some applications, filter chambers 510A and 510B further comprise one or more filter supports 530, which are disposed within the filter chambers, and are shaped so as to define respective (a) proximal support surfaces that mechanically support respective filters, and (b) respective pluralities of filter-support openings through the filter supports to allow passage of filtrate from the respective filters.

For some applications, the filter unit further comprises a liquid-pressure source 534, which is arranged to apply pressure to drive the liquid specimen sample through the one or more filters. For example, liquid-pressure source 534 may comprise a pump 536, such as shown, by way of example, for filter unit 500A, or a plunger 538, such as shown, by way of example, for filter unit 500B.

For some applications, the filter unit further comprises a waste receptacle 540, which is disposed in fluid communication with outlet 514 of the filter chamber so as to receive filtrate from the one or more filters. For some of these applications, waste receptacle 540 cannot be removed from the filter chamber without damaging the filter unit, such as shown. For other applications, waste receptacle 540 is configured to be decoupled from the filter chamber without damaging the filter unit (configuration not shown).

For some applications, waste receptacle 540 contains a disinfectant 544, such as shown, by way of example, for filter unit 500A, or a liquid-absorbing material 546, such as shown, by way of example, for filter unit 500B.

As described in the section entitled, “Experiment 7,” in the above-mentioned U.S. application Ser. No. 17/921,672 and International Application PCT/IL2021/050519, the inventors found that filters having pore sizes substantially larger than the viral diameter were nevertheless able to trap virus in the saliva of gargled fluid in quantities sufficient to clinically identify the presence of the virus, even using immunoassays, which generally require substantially greater quantities of virus for detection than does PCR. The inventors hypothesize that the large-pore filters were able to trap virus at least in part because of the viscosity of the saliva in typical gargled fluid samples, perhaps in particular because of the viscosity of the mucus and/or other large or aggregated salivary components of the saliva, which themselves contain viral particles, and/or because the viral particles adhered (e.g., directly adhered) to the material of the large-pore filters. Use of large-pore filters may enable a high flow rate and inhibit filter clogging despite high sample viscosity.

For some applications, the challenge-test nominal pore size is at least 40 microns. For some applications, the challenge-test nominal pore size is at least 50 microns. For some applications, the challenge-test nominal pore size is at least 60 microns. For some applications, the challenge-test nominal pore size is at least 100 microns. For some applications, the challenge-test nominal pore size is at least 120 microns. For some applications, the challenge-test nominal pore size is at least 150 microns. For some applications, the challenge-test nominal pore size is at least 200 microns. For some applications, the challenge-test nominal pore size is at least 500 microns.

For some applications, the challenge-test nominal pore size is less than 1 mm. For some applications, the challenge-test nominal pore size is less than 750 microns. For some applications, the challenge-test nominal pore size less than 500 microns. For some applications, the challenge-test nominal pore size less than 250 microns. For some applications, the challenge-test nominal pore size less than 200 microns.

For some applications, the challenge-test nominal pore size is between 30 and 40 microns. For some applications, the challenge-test nominal pore size is between 40 and 60 microns. For some applications, the challenge-test nominal pore size is between 50 and 200 microns, such as between 50 and 150 microns, e.g., between 100 and 150 microns. For some applications, the challenge-test nominal pore size is between 60 and 100 microns. For some applications, the challenge-test nominal pore size is between 100 and 120 microns. For some applications, the challenge-test nominal pore size is between 120 and 150 microns. For some applications, the challenge-test nominal pore size is between 150 and 200 microns. For some applications, the challenge-test nominal pore size is between 200 and 500 microns. For some applications, the challenge-test nominal pore size is between 500 microns and 1 mm, such as between 500 and 750 microns, or between 750 microns and 1 mm.

For some applications, the bubble-point nominal pore size is at least 40 microns. For some applications, the bubble-point nominal pore size is at least 50 microns. For some applications, the bubble-point nominal pore size is at least 60 microns. For some applications, the bubble-point nominal pore size is at least 100 microns. For some applications, the bubble-point nominal pore size is at least 120 microns. For some applications, the bubble-point nominal pore size is at least 150 microns. For some applications, the bubble-point nominal pore size is at least 200 microns.

For some applications, the bubble-point nominal pore size less than 500 microns. For some applications, the bubble-point nominal pore size less than 300 microns. For some applications, the bubble-point nominal pore size less than 250 microns. For some applications, the bubble-point nominal pore size less than 200 microns.

For some applications, the bubble-point nominal pore size is between 30 and 40 microns. For some applications, the bubble-point nominal pore size is between 40 and 60 microns. For some applications, the bubble-point nominal pore size is between 50 and 200 microns, such as between 50 and 150 microns, e.g., between 100 and 150 microns. For some applications, the bubble-point nominal pore size is between 60 and 100 microns. For some applications, the bubble-point nominal pore size is between 100 and 120 microns. For some applications, the bubble-point nominal pore size is between 120 and 150 microns. For some applications, the bubble-point nominal pore size is between 150 and 200 microns. For some applications, the bubble-point nominal pore size is between 200 and 300 microns.

Typically, the method does not comprise centrifuging the liquid specimen sample.

For some applications, the filter is one of a plurality of filters arranged in a series of filters (such as described hereinabove with reference to FIG. 8B). For some applications, the filters have respective challenge-test nominal pore sizes, all of which are between 30 microns and 1.5 mm, or any of the challenge-test nominal pore sizes provided above. For some applications, the filters have respective bubble-point nominal pore sizes, all of which are between 30 and 500 microns, such as between 30 and 300 microns, or any of the bubble-point nominal pore sizes provided above. The method comprises passing the liquid specimen sample through the series of filters, without passing the liquid specimen sample through any other filters. Deriving the information from the extracellular vesicles present in the saliva comprises deriving the information from the extracellular vesicles present in the saliva trapped by at least one of the plurality of filters, such as deriving the information from the extracellular vesicles present in the saliva trapped by at least two of the plurality of filters.

For some applications, the filter is a first filter (such as large-pored filter 520 of filter chamber 510B, described hereinabove with reference to FIG. 8B). Passing the liquid specimen sample through the filter comprises passing the liquid specimen sample through the first filter to produce a filtrate. Deriving the information from the extracellular vesicles present in the saliva trapped by the filter comprises deriving the information from the extracellular vesicles present in the saliva trapped by the first filter. The method further comprises passing the filtrate through a second filter (such as additional filter 522 of filter chamber 510B, described hereinabove with reference to FIG. 8B) to trap at least some of the saliva, and deriving the information from the extracellular vesicles present in the saliva trapped by the second filter. For some of these applications, a nominal pore size of the second filter equals the nominal pore size of the first filter. For some of these applications, a challenge-test nominal pore size of the second filter is between 0.03 and 21 microns, such as between 0.8 and 1.5 microns. For some of these applications, a bubble-point nominal pore size of the second filter is between 0.03 and 21 microns, such as between 0.8 and 1.5 microns. For some of these applications, the second filter may be configured (such as by its nominal pore size) to capture epithelial cells.

For some applications, passing the liquid specimen sample through the filter comprises passing the liquid specimen sample through the filter to trap at least some of the saliva by size-based filtration.

For some applications, passing the liquid specimen sample through the filter comprises passing the liquid specimen sample through the filter to trap at least some of the saliva by adhesion of the saliva to the filter.

For some applications, the filter comprises a depth filter.

For some applications, the filter comprises a mesh filter, such as a woven mesh filter or a non-woven mesh filter.

For some applications, the filter comprises fibers, such as crisscrossing fibers.

For some applications, the filter comprises a screen, such as a woven screen or a non-woven screen.

For some applications, the filter comprises a polymer, such as a polyester.

For some applications, the filter comprises a metal.

For some applications, passing the liquid specimen sample through the filter comprises passing the liquid specimen sample through exactly one filter.

For some applications, collecting the liquid specimen sample comprises receiving the liquid specimen sample by a healthcare worker from the subject.

For some applications, the method does not comprise swabbing nasal cavity 22. Alternatively or additionally, for some applications, the method does not comprise swabbing a pharynx of the subject.

For some applications, the method does not comprise swabbing any portion of the subject. Alternatively, the method comprises swabbing a portion of the subject, such as using swabbing techniques described herein.

For some applications, collecting the liquid specimen sample comprises collecting the liquid specimen sample that was expressed out of oropharynx 30 via anterior opening 34 of oral cavity 36. For some of these applications, collecting the liquid specimen sample comprises collecting the liquid specimen sample that was spit out of oropharynx 30 via anterior opening 34 of oral cavity 36.

For some applications, collecting the liquid specimen sample comprises aspirating the liquid specimen sample out of oropharynx 30 via anterior opening 34 of oral cavity 36.

For some applications, collecting the liquid specimen sample comprises:
gargling, by the subject, oral wash fluid 68 to produce the liquid specimen sample that includes the saliva; and
thereafter, collecting the liquid specimen sample that passed out of anterior opening 34 of oral cavity 36.

For some applications, collecting the liquid specimen sample comprises:
gargling, by the subject, oral wash fluid 68 to produce the liquid specimen sample that includes the saliva; and
thereafter, expressing, by the subject, out of anterior opening 34 of oral cavity 36, the liquid specimen sample.

For some applications, collecting the liquid specimen sample comprises:
swishing, by the subject, oral wash fluid 68 in oral cavity 36 to produce the liquid specimen sample that includes the oral wash fluid and the saliva; and
thereafter, collecting the liquid specimen sample from the subject.

For some applications, collecting the liquid specimen sample comprises:
swishing, by the subject, oral wash fluid 68 in oral cavity 36 to produce the liquid specimen sample that includes the oral wash fluid and the saliva; and
thereafter, expressing, by the subject, out of anterior opening 34 of oral cavity 36, the liquid specimen sample.

For some applications, collecting the liquid specimen sample comprises:
spraying, via anterior opening 34 of oral cavity 36, oropharynx 30 with oral wash fluid 68; and
thereafter, collecting the liquid specimen sample from the subject.

For some applications, the above-mentioned gargling, swishing, and/or spraying techniques are combined.

For some applications, collecting the gargled fluid from the subject comprises:
intranasally dispensing nasal wash fluid 26 into nasal cavity 22 such that nasal wash fluid 26 washes biological material into oropharynx 30 from (a) nasal cavity 22, (b) nasopharynx 32, or (c) nasal cavity 22 and nasopharynx 32; and
thereafter, collecting the liquid specimen sample that passed out of anterior opening 34 of oral cavity 36 and includes oral wash fluid 68, nasal wash fluid 26, and at least a portion of the biological material washed into oropharynx 30 by nasal wash fluid 26.

For some applications, collecting the liquid specimen sample comprises expressing, by the subject, the liquid specimen sample out of oropharynx 30 via anterior opening 34 of oral cavity 36. For some applications, expressing the liquid specimen sample comprises spitting, by the subject, the liquid specimen sample out of oropharynx 30 via anterior opening 34 of oral cavity 36.

For some applications, collecting the liquid specimen sample comprises:
after intranasally dispensing nasal wash fluid 26, orally dispensing oral wash fluid into oral cavity 36 via anterior opening 34 of oral cavity 36 such that oral wash fluid 68 forms mixture 70 with nasal wash fluid 26 in oropharynx 30;
gargling, by the subject, mixture 70 to produce the liquid specimen samples that includes oral wash fluid 68, nasal wash fluid 26, and the saliva; and
collecting the liquid specimen sample that passed out of anterior opening 34 of oral cavity 36.

For some applications, collecting the liquid specimen sample from the subject comprises:
intranasally dispensing nasal wash fluid into nasal cavity 22 such that nasal wash fluid 26 washes biological material into an oropharynx of the subject from (a) nasal cavity 22, (b) a nasopharynx of the subject, or (c) nasal cavity 22 and the nasopharynx; and
thereafter, expressing, by the subject, out of anterior opening 34 of oral cavity 36, the liquid specimen sample that includes at least a portion of the biological material washed into oropharynx 30 by nasal wash fluid 26.

For some applications, expressing the liquid specimen sample comprises spitting, by the subject, the liquid specimen sample out of oropharynx 30 via anterior opening 34 of oral cavity 36.

For some applications, collecting the liquid specimen sample comprises:
after intranasally dispensing nasal wash fluid 26, orally dispensing oral wash fluid 68 into oral cavity 36 via anterior opening 34 of oral cavity 36 such that oral wash fluid 68 forms mixture 70 with nasal wash fluid 26 in oropharynx 30;
gargling, by the subject, mixture 70 to produce the liquid specimen sample that includes oral wash fluid 68, nasal wash fluid 26, and the saliva; and
expressing, by the subject, the liquid specimen sample out of anterior opening 34 of oral cavity 36.

For some applications, the subject intranasally dispenses nasal wash fluid 26 into nasal cavity 22, while for some other applications, a healthcare worker intranasally dispenses nasal wash fluid 26 into nasal cavity 22.

For some applications, the method further comprises, after intranasally dispensing nasal wash fluid 26, gargling, by the subject, nasal wash fluid 26 washed into oropharynx 30.

Any of the method described herein that comprise collecting, filtering, and testing a liquid specimen sample that includes saliva and orally-dispensed oral wash fluid 68 may alternatively comprise collecting, filtering, and testing a liquid specimen sample that includes (a) a biological material other than saliva and (b) orally-dispensed oral wash fluid 68.

For some applications, the techniques described herein are practiced in combination with techniques described for the nasal wash fluid dispensers described with reference to (a) FIGS. 6 and 7A-K, (b) FIGS. 8A-C, and/or (c) FIGS. 9A-F, 10, and 11, all of the above-mentioned U.S. application Ser. No. 17/921,672 and International Application PCT/IL2021/050519.

### EXPERIMENTAL RESULTS

The inventors performed a number of experiments using some of the techniques described herein, including Experiments 1, 2, 3, 4, 5, 6, 7, and 8 described in the above-mentioned U.S. application Ser. No. 17/921,672 and International Application PCT/IL2021/050519. Although many of the experiments described in these two patent applications include testing for the presence of the SARS-CoV-2 virus, the inventors believe that the testing techniques used in these experiments would provide similar results for other viruses sharing one or more characteristics with SARS-CoV-2, such as infection of the nasal cavity and viral tropism. Such other viruses include, for example, Influenza viruses, and rhinoviruses.

For example, there is shared tropism in Influenza and SARS-CoV-2 infection. The expression of receptors used in cellular entry is one of the principal limitations on viral tropism, with SARS-CoV-2 and Influenza requiring Angiotensin-converting enzyme-2 (ACE2) and Sialic acid (SA) expression, respectively, to be permissive for infection. The replication of SARS-CoV-2 in upper respiratory tract (URT) tissues is attributed to the expression of ACE2 in olfactory and respiratory epithelial c ells. Similarly, the permissiveness of human nasal epithelial cells to Influenza infection in vitro is believed to be due to the cell-surface expression of SA in ciliated epithelial cells. Moreover, SARS-CoV-2 and Influenza have been reported to have similar tissue tropism in the lungs and bronchus. The inventors therefore believe that sampling methods described herein, such as nasal saline spraying and swabbing or nasal saline spraying and gargling, which produce samples with higher SARS-CoV-2 viral loads and/or amounts of infected cell debris than traditional sampling methods, would similarly be a more effective sampling procedure for the diagnosis and/or detection of Influenza or any other URT infection which reproduce in nasal epithelial cells, such as human rhinoviruses.

The inventors also believe that the sample collection testing techniques used in the above-mentioned experiments described in above-mentioned U.S. application Ser. No. 17/921,672 and International Application PCT/IL2021/050519 would provide similar results for collecting specimen samples for deriving information from extracellular vesicles present in the specimen samples.

### Experiment 1

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Experiment 1 provides evidence of the efficacy of these techniques for collecting specimen samples for detection of SAILS-CoV-2 using quantitative PCR (qPCR).

In summary, Experiment 1 found that spraying saline solution into the nasal cavity and gargling resulted in between a 3.4-fold (340%), and 5.6-fold (560%) increase in viral material after nasal saline spraying and gargling compared to before nasal saline spraying and gargling, depending on the gene target, after a nucleic acid extraction protocol consisting of including heating samples. Experiment 1 also found that spraying saline solution into the nasal cavity and gargling resulted in between a 2.7-fold (270%) and 8.7-fold (870%) increase in viral material after nasal saline spraying and gargling compared to before nasal saline spraying and gargling, depending on the gene target, after a robust magnetic bead based nucleic acid extraction protocol.

### Detailed Report of Experiment 1: Benefits of Nasal Saline Spraying Before Anterior Nares Swabbing in SARS-CoV-2 Detection

### Section 1: Materials and Methods

### Section 1.1: Subject Sampling

Bilateral anterior nares swabs were performed by a healthcare professional on 18 subjects belonging to the health care service provider Leumit (Tel-Aviv, Israel), who had previously tested positive for SARS-CoV-2. The subjects then sprayed 0.1 mL of normal saline 3-5 times into each nostril (some while their heads were tilted back and others while their heads were in a neutral position), and then gargled 10 mL of normal saline for 10-15 seconds to produce a Nasal Gargle (hereinbelow, “NG”) specimen. Following spitting out of the NG sample, a second bilateral anterior nares swab was performed by the healthcare professional, while the subjects' heads were in a neutral position or tilted slightly back. Swabs were then placed into tubes containing 3 mL of Universal Transport Medium (UTM) (Biological Industries, Israel). Two subjects were excluded from the study due to an inability to gargle. For 3 subjects (Subjects 5-7), a bilateral mid-turbinate swab was performed by the healthcare professional prior to the production of the NG sample. Bilateral anterior nares swabbing was performed by inserting swabs into the nares and then removing the swabs without rotating the swabs against the inner wall of the nares. Samples were transported on ice to either the Leumit Health Services Central Laboratory (Or Yehuda, Israel), or to Hero Scientific Ltd. (Jerusalem, Israel) for processing. The clinical study was approved by the investigational review board (hereinbelow, “IRB”) at Assaf Harofeh (Shamir) Medical Center (Tel Aviv, Israel).

It is important to note that the anterior nares swab sample collection was performed by inserting the swab about 1.5 cm into the nostril and spinning the swab vertically as is typically performed for a mid-turbinate swab sample collection. All anterior nares swab sample collection procedures performed for all other experiments described herein (Experiments 2, 3, 4, 5, 6, and 7) were performed by inserting the swab about 1.5 cm into the nostril and rubbing the swab in a circular motion so as to abrasively rub against the wall of the anterior naris. The inventors hypothesize that this experiment may have been negatively impacted by this non-ideal anterior nares swab sample collection procedure. In particular, the anterior nares swab sample performed after saline nasal administration may have been unable to properly collect the saline dripping down the wall of the anterior naris and thus not yield the benefits of saline nasal administration in increasing the viral load collected. Conversely, the inventor hypothesize that other experiments described herein (Experiments 2, 3, 4, 5, 6, and 7) are more adequate representations of the benefit of anterior nares swab sampling after saline nasal administration.

### Section 1.2: Molecular Testing

Section 1.2.1: qPCR Following Heat Extraction

Samples from 14 of the 16 subjects were processed by quantitative reverse transcription polymerase chain reaction (hereinbelow, “qPCR”) at Hero Scientific Ltd. (Jerusalem, Israel). Samples were first inactivated by boiling at 70° C. for 45 minutes in a heat block with aluminum beads. All samples were processed using the primers and probe sequences from the Center for Disease Control's (hereinbelow, “CDC”) diagnostic panel provided in the 2019-nCoV RUO Kit (CAT #10006713, IDT, Belgium) on an MX3000P (Stratagene, USA) qPCR thermocycler. The reaction mixture for samples from 3 of the 14 subjects (Subjects 5-7) were prepared using the GE50 Xpert One-Step Fast Probe (GRiSP Research Solutions, Portugal), and contained 10 microliters of Fast qPCR Mastermix (GRiSP Research Solutions, Portugal), 0.5 microliters of RTase Mix (GRiSP Research Solutions, Portugal), 2 microliters of primer mix (CAT #10006713, IDT, Belgium), 4 microliters of inactivated sample, and 3.5 microliters of Molecular Grade Water (Biological Industries, Israel). Negative template controls (hereinbelow, “NTCs”) were performed by testing 4 microliters of Molecular Grade Water (Biological Industries, Israel) instead of the samples for all primer/probe mixtures tested. Similarly, positive controls were performed by testing 1 microliter of positive control plasmid, 2019-nCoV N Positive Control (CAT #10006625, IDT, Belgium), and 3 microliters of Molecular Grade Water (Biological Industries, Israel) instead of the samples for all primer/probe mixtures tested. The thermal profile conditions were 45° C. for 15 minutes, 95° C. for 3 minutes, and then 40 cycles of 95° C. for 5 seconds and 58° C. for 30 seconds. Prior to use of the GE50 Xpert One-Step Fast Probe, 2 microliters of 100×ROX was added to the Fast qPCR Mastermix (Probe) as per the manufacturer's instructions, and cycle threshold (hereinbelow, “Ct”) values were normalized to ROX. (As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels.)

Samples from 12 of the 14 subjects (including Subjects 1-4 and 8-11) were processed using the PrimeDirect Probe RT-qPCR Mix (Takara Bio inc., Japan). Reaction mixtures contained 12.5 microliters of the PrimeDirect Probe RT-qPCR Mix (Takara Bio inc., Japan), 4 microliters of sample, 2 microliters of the primer/probe mix (IDT, Belgium), and 6.5 microliters of Molecular Grade Water (Biological Industries, Israel). NTCs were performed for each qPCR run by testing 4 microliters of Molecular Grade Water instead of the samples for all primer/probe mixtures tested. Similarly, positive controls were performed by testing 1 microliter of positive control plasmid, 2019-nCoV N Positive Control (CAT #10006625, IDT, Belgium), and 3 microliters of Molecular Grade Water (Biological Industries, Israel) instead of the samples for all primer/probe mixtures tested. The thermal profile conditions were 90° C. for 3 minutes, 60° C. for 5 minutes, and then 40 cycles of 95° C. for 5 seconds and 60° C. for 30 seconds.

Section 1.2.2: qPCR Following Magnetic Bead Extraction

Samples from 2 of the 16 subjects (Subjects 12 and 13) were processed by qPCR at the Leumit Health Services Central Laboratory (Or Yehuda, Israel) using the Allplex 2019 n-CoV Assay (Seegene, Seoul, South Korea), following manufacturer's instructions, on a CFX96 Real-Time System (BIO RAD, USA). Prior to qPCR, samples were extracted using the STARMag 96×4 Viral DNA/RNA 200 C Kit (Seegene, Seoul, South Korea) on a MICROLAB STAR^{let} (Hamilton, Switzerland) automated liquid handling platform.

### Section 2: Results and Discussion

Out of the 16 subjects, 13 (n=13) were found to be positive for SARS-CoV-2 on at least one gene target for either of the paired anterior nares swabs. Average Ct value differences between (a) anterior nares swabs sampled before nasal saline spraying and (b) anterior nares swabs samples after nasal saline spraying and gargling for SARS-CoV-2 gene targets, after a nucleic acid extraction protocol including heating samples, ranged from −1.75 to −2.5, corresponding to between a 3.4-fold (340%), and 5.6-fold (560%) increase in viral material after nasal saline spraying and gargling compared to before nasal saline spraying and gargling, depending on the gene target (Table 4.1). Similarly, average ΔCt value differences between (a) anterior nares swabs sampled before nasal saline spraying and (b) anterior nares swabs samples after nasal saline spraying and gargling for SARS-CoV-2 gene targets, after a robust magnetic bead based nucleic acid extraction protocol, ranged from −1.45 to −3.12, corresponding to between a 2.7-fold (270%) and 8.7-fold (870%) increase in viral material after nasal saline spraying and gargling compared to before saline nasal spraying and gargling, depending on the gene target (Table 4.2). This phenomenon is likely due to the saline introduced during NG sampling dripping from within the nasal cavity back into the anterior nares, increasing the amount of viral material available for subsequent swabbing. It is therefore expected that performing nasal spray prior to anterior nares swabbing would lead to an increase in sensitivity in any downstream molecular or immunological tests performed using the swabbed specimen samples. Interestingly, similar results were observed across different RNA extraction and qPCR protocols, indicating that a combined nasal spray and anterior nares swab sampling methodology would be effective at increasing diagnostic sensitivity even when used in conjunction with devices where less-robust nucleic acid extraction methods are employed.

TABLE 4.1
A Comparison Between Anterior Nares Swabbins before and
after Nasal Gargling following Heat Extraction

| | | 1^{st} | 2^{nd} | | |
|---|---|---|---|---|---|
| | | Anterior | Anterior | | |
| | | Nares | Nares | | Average Ct |
| Gene | Subject | Swab Ct | Swab Ct | Ct Value | Value |
| Target | # | Valueⁱ | Valueⁱ | Differenceⁱⁱ | Differenceⁱⁱ |
| N1 | 1 | 32.73 | 34.84 | 2.11 | −1.7536364 |
| | 2 | 34.91 | 40 | 5.09 | |
| | 3 | 39.05 | 31.84 | −7.21 | |
| | 4 | 40 | 40 | 0 | |
| | 5ⁱⁱⁱ | 31.7 | 24.15 | −7.55 | |
| | 6ⁱⁱⁱ | 17.95 | 26.54 | 8.59 | |
| | 7ⁱⁱⁱ | 31.27 | 24.14 | −7.13 | |
| | 8 | 39.52 | 38.2 | −1.32 | |
| | 9 | 27.62 | 27.92 | 0.3 | |
| | 10 | 40 | 34.23 | −5.77 | |
| | 11 | 31.98 | 25.58 | −6.4 | |
| N2 | 1 | 40 | 40 | 0 | −2.4954545 |
| | 2 | 40 | 40 | 0 | |
| | 3 | 40 | 31.72 | −7.01 | |
| | 4 | 40 | 39.23 | −0.77 | |
| | 5ⁱⁱⁱ | 36.58 | 25.06 | −11.52 | |
| | 6ⁱⁱⁱ | 19.69 | 28.81 | 9.12 | |
| | 7ⁱⁱⁱ | 34.53 | 26.84 | −7.69 | |
| | 8 | 40 | 38.68 | −1.32 | |
| | 9 | 32.81 | 29.32 | −3.49 | |
| | 10 | 40 | 38.62 | −1.38 | |
| | 11 | 33.69 | 30.3 | −3.39 | |

ⁱNegative Ct values, corresponding to a value of ≥40, were assigned an actual value of 40.
ⁱⁱIn instances where both swabs were negative, the average Ct value difference was assumed to be 0.
ⁱⁱⁱThe NTC accompanying the tests for Subjects 5-7 had evidence of human DNA contamination. However, if human DNA contamination were to occur in subject samples, it would be unlikely to affect results for SARS-CoV-2 specific gene targets, and so the results were included, but not normalized to the reference gene, RNase P.

TABLE 4.2
A Comparison Between Anterior Nares Swabbins before and after Nasal
Gargling Following Magnetic Bead Extraction

| | | 1^{st} | 2^{nd} | Normalized | Normalized | | |
|---|---|---|---|---|---|---|---|
| | | Anterior | Anterior | 1^{st} Anterior | 2^{nd} Anterior | Normalized | Average |
| | | Nares | Nares | Nares | Nares | ΔCt | ΔCt |
| Gene | Subject | Swab Ct | Swab Ct | Swab ΔCt | Swab ΔCt | Value | Value |
| Target | # | Value | Value | Valueⁱ | Valueⁱ | Differenceⁱⁱ | Difference |
| E | 12 | 24.83 | 26.72 | −1.4 | 0.23 | 1.63 | −1.73 |
| | 13 | 35.04 | 29.57 | 8.5 | 3.41 | −5.09 | |
| RdRP/S | 12 | 26.93 | 28.55 | 0.7 | 2.06 | 1.36 | −3.12 |
| | 13 | 39.19 | 31.21 | 12.65 | 5.05 | −7.6 | |
| N | 12 | 27.77 | 29.42 | 1.54 | 2.93 | 1.39 | −1.45 |
| | 13 | 37.16 | 32.49 | 10.62 | 6.33 | −4.29 | |

ⁱNormalized ΔCt value is calculated by subtracting the Ct value of a reference gene from the Ct value of the target gene.
ⁱⁱNormalized ΔCt value difference is calculated by subtracting the normalized ΔCt value of the anterior nares swab performed prior to the NG (1^{st}) from the normalized ΔCt value of the anterior nares swab performed after the NG (2^{nd}).

In some applications, any of the techniques described in the report of Experiment 1 above are used in combination with the other techniques described herein.

### Experiment 2

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using PCR. Experiment 2 provides evidence of the efficacy of these techniques for collecting specimen samples for detection of SARS-CoV-2 using quantitative PCR (qPCR).

In summary, Experiment 2 found that eleven out of 15 subjects tested (73.3%) had lower normalized cycle threshold (Ct) values in samples from anterior nares swabs performed after nasal saline spraying and gargling than from anterior nares swabs performed before nasal saline spraying and gargling. Furthermore, 7 out of the 15 subjects (46.7%) were found to have lower normalized Ct values in samples from anterior nares swabs performed after nasal saline spraying and gargling than in mid-turbinate swabs. In cases where the mid-turbinate swab outperformed the anterior nares swabs sampled after nasal saline spraying and gargling, anterior nares swabs sampled after nasal saline spraying and gargling were still within 1 ΔCt value of the mid-turbinate swab in 5 out 8 subjects (62.5%).

### Detailed Report of Experiment 2: Benefits of Nasal Saline Spraying Prior to Anterior Nares Swabbing in SARS-CoV-2 Detection on the Cobas 6800 System

### Section 1: Materials and Methods

### Section 1.1: Subject Sampling

Bilateral anterior nares swabbing was performed by a healthcare professional on 15 subjects belonging to the health care service provider Leumit (Tel-Aviv, Israel), who had previously tested positive for SARS-CoV-2. A bilateral mid-turbinate swab was then performed by the healthcare professional. The subjects then sprayed 0.1 mL of normal saline 3-5 times into each nostril, and then gargled 10 mL of normal saline for 10-15 seconds to produce a Nasal Gargle (hereinbelow, “NG”) specimen. Following spitting out of the NG sample, a second bilateral anterior nares swab was performed by the healthcare professional. Swabs were then placed into tubes containing 3 mL of Universal Transport Medium (UTM) (Biological Industries, Israel). Samples were transported on ice to the Leumit Health Services Central Laboratory (Or Yehuda, Israel), for processing. The clinical study was approved by the investigational review board (IRB) at Assaf Harofeh (Shamir) Medical Center (Tel Aviv, Israel). All swabbing was performed with flocked swabs (3BY Ltd., Industrial Park Tefen, Ref #: 3by-001).

### Section 1.2: Molecular Testing

RNA extraction and qPCR were performed using the Cobas SARS-CoV-2 Test (Roche, Switzerland) on a Cobas 6800 system (Roche, Switzerland), in accordance with manufacturer instructions.

### Section 2: Results and Discussion

All 15 subjects were positive for both ORF1 a/b and E gene targets (Table 5). (As is known in the quantitative PCR art, the Ct value is defined as the number of cycles of amplification (using PCR) required for the fluorescence of a PCR product (i.e., the target/amplicon) to be detected crossing a threshold that is above the background signal. Relatively lower Ct values indicate higher initial viral levels.)

Fourteen out of the 15 subjects (93.3%) were found to have lower Ct values, indicative of higher viral levels, for both gene targets in samples from anterior nares swabs performed after nasal saline spraying and gargling than from anterior nares swabs performed before nasal saline spraying and gargling. Furthermore, 7 out of the 15 subjects (46.7%) were found to have lower Ct values, indicative of higher viral levels, across both gene targets in samples from anterior nares swabs performed after nasal saline spraying and gargling than in mid-turbinate swabs. In cases where the mid-turbinate swab outperformed the anterior nares swabs sampled after nasal saline spraying and gargling on at least one gene target, anterior nares swabs sampled after nasal saline spraying and gargling were still within 1 Ct value of the mid-turbinate swab in 4 out 8 subjects (62.5%), with 2 of the subjects outperforming mid-turbinate swabs on the second target.

On average, Ct values for anterior nares swabs performed after nasal saline spraying decreased by 2.35 for the ORF1 a/b gene and by 2.70 for the E gene compared to Ct values for anterior nares swabs performed before nasal saline spraying (Table 6). This decrease was statistically significant (ORF1 a/b gene, p=0.00014; E gene, p=0.00125) (Student's t-test).

This data suggest that dispensing nasal spray and gargling before anterior nares swabbing increases collected viral material when compared to anterior nares swabs without a prior nasal spray and gargle, and in some cases, when compared to mid-turbinate swabs. It is therefore expected that administering a nasal spray prior to anterior nares swabbing as a sampling method would result in an increase in molecular and immunological testing sensitivity.

TABLE 5
Ct value results

| Subject | Sampleⁱ | ORF1 a/b gene | E gene | ICⁱⁱ |
|---|---|---|---|---|
| 1 | AN1 | 20.38 | 20.45 | 32.91 |
| | MT | 19.53 | 19.66 | 33.12 |
| | AN2 | 19.42 | 19.66 | 32.9 |
| 2 | AN1 | 34.05 | 39.74 | 32.83 |
| | MT | 30.8 | 31.6 | 33.04 |
| | AN2 | 29.78 | 30.51 | 32.81 |
| 3 | AN1 | 24.36 | 24.14 | 32.33 |
| | MT | 23.27 | 23.27 | 32.68 |
| | AN2 | 23.32 | 23.14 | 32.59 |
| 4 | AN1 | 32.73 | 33.79 | 33.13 |
| | MT | 28.71 | 29.19 | 32.75 |
| | AN2 | 30.54 | 31.35 | 32.77 |
| 5 | AN1 | 27.04 | 27.1 | 32.27 |
| | MT | 27.51 | 27.7 | 32.68 |
| | AN2 | 28.11 | 28.51 | 32.59 |
| 6 | AN1 | 26.62 | 26.87 | 32.68 |
| | MT | 25.24 | 25.46 | 32.53 |
| | AN2 | 23.87 | 24.07 | 32.42 |
| 7 | AN1 | 29.89 | 30.44 | 32.89 |
| | MT | 24.82 | 24.76 | 32.35 |
| | AN2 | 26.89 | 27.24 | 32.55 |
| 8 | AN1 | 28.19 | 28.54 | 32.66 |
| | MT | 29.52 | 30.1 | 32.76 |
| | AN2 | 26.43 | 26.64 | 32.23 |
| 9 | AN1 | 21.24 | 21.48 | 32.59 |
| | MT | 20.98 | 21.29 | 32.91 |
| | AN2 | 19.09 | 19.1 | 33.5 |
| 10 | AN1 | 30.05 | 30.69 | 33.11 |
| | MT | 30.17 | 30.99 | 33.11 |
| | AN2 | 29.12 | 29.96 | 32.72 |
| 11 | AN1 | 34.26 | 35.8 | 32.82 |
| | MT | 29.5 | 30.48 | 33.38 |
| | AN2 | 29.07 | 30.01 | 32.73 |
| 12 | AN1 | 30.62 | 31.18 | 32.89 |
| | MT | 23.37 | 23.59 | 32.7 |
| | AN2 | 25.17 | 25.7 | 34.19 |
| 13 | AN1 | 30.04 | 30.43 | 32.94 |
| | MT | 27.27 | 27.57 | 32.61 |
| | AN2 | 27.67 | 27.97 | 32.37 |
| 14 | AN1 | 27.84 | 28.34 | 32.7 |
| | MT | 27.03 | 27.64 | 32.81 |
| | AN2 | 24.4 | 24.88 | 32.76 |
| 15 | AN1 | 35.18 | 36.79 | 32.89 |
| | MT | 33.02 | 34.4 | 32.82 |
| | AN2 | 34.33 | 36.49 | 32.73 |

AN1 = anterior nares swab performed prior to nasal saline spraying and gargling,
MT = mid-turbinate swab,
AN2 = anterior nares swab performed after nasal saline spraying and gargling
IC = internal control

TABLE 6
Average Ct values and Average Difference between AN1 and AN2

| | AN1 | | | | | |
|---|---|---|---|---|---|---|
| | ORF | | AN2 | | Delta: AN2-AN1 | |
| | gene | E | ORF1 a/b | E | ORF1 a/b | E |
| Subject | 1 a/b | gene | gene | gene | gene | gene |
| 1 | 20.38 | 20.45 | 19.42 | 19.66 | −0.96 | −0.79 |
| 2 | 34.05 | 39.74 | 29.78 | 30.51 | −4.27 | −9.23 |
| 3 | 24.36 | 24.14 | 23.32 | 23.14 | −1.04 | −1 |
| 4 | 32.73 | 33.79 | 30.54 | 31.35 | −2.19 | −2.44 |
| 5 | 27.04 | 27.1 | 28.11 | 28.51 | 1.07 | 1.41 |
| 6 | 26.62 | 26.87 | 23.87 | 24.07 | −2.75 | −2.8 |
| 7 | 29.89 | 30.44 | 26.89 | 27.24 | −3 | −3.2 |
| 8 | 28.19 | 28.54 | 26.43 | 26.64 | −1.76 | −1.9 |
| 9 | 21.24 | 21.48 | 19.09 | 19.1 | −2.15 | −2.38 |
| 10 | 30.05 | 30.69 | 29.12 | 29.96 | −0.93 | −0.73 |
| 11 | 34.26 | 35.8 | 29.07 | 30.01 | −5.19 | −5.79 |
| 12 | 30.62 | 31.18 | 25.17 | 25.7 | −5.45 | −5.48 |
| 13 | 30.04 | 30.43 | 27.67 | 27.97 | −2.37 | −2.46 |
| 14 | 27.84 | 28.34 | 24.4 | 24.88 | −3.44 | −3.46 |
| 15 | 35.18 | 36.79 | 34.33 | 36.49 | −0.85 | −0.3 |
| Average | 28.83 | 29.72 | 26.48 | 27.01 | −2.35 | −2.70 |

In some applications, any of the techniques described in the report of Experiment 2 above are used in combination with the other techniques described herein. It is noted that although Experiment 2, as reported above, included mid-turbinate swabbing of the subjects, this mid-turbinate swabbing served for comparison with anterior nares swabbing. Some techniques of the present invention include performing only an anterior nares swab, without performing a mid-turbinate swab.

### Experiment 3

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay. Experiment 3 provides evidence of the efficacy of these techniques for collecting specimen samples for detection of SARS-CoV-2 using lateral flow immunoassay strips.

In summary, Experiment 3 found that two of the four subjects tested positive for SARS-CoV-2 antigens both on the mid-turbinate swab and on the anterior nares swab performed after nasal saline spraying, but negative on the anterior nares swab performed before nasal saline spraying, indicating that nasal saline spraying can increase anterior nares swab sensitivity with immunoassays. The remaining two of the four subjects tested negative for SARS-CoV-2 antigens on all sample types (anterior nares swabbing before nasal saline spraying, mid-turbinate swabbing, and anterior flares swabbing after nasal saline spraying).

### Detailed Report of Experiment 3: Benefits of Nasal Saline Spraying Prior to Anterior Nares Swabbing in SARS-CoV-2 Detection by Immunoassay

### Section 1: Materials and Methods

### Section 1.1: Subject Sampling

Bilateral anterior nares swabbing was performed by a healthcare professional on 20 subjects belonging to the health care service provider Leumit (Tel-Aviv, Israel), who had previously tested positive for SARS-CoV-2. The subjects then sprayed 0.1 mL of normal saline 3-5 times into each nostril, and then gargled 10 mL of normal saline for 10-15 seconds to produce a Nasal Gargle specimen. Following spitting out of the Nasal Gargle sample, a second bilateral anterior nares swab was performed by the healthcare professional. Lastly, a bilateral mid-turbinate swab was performed by the healthcare professional. Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001). The clinical study was approved by the investigational review board (IRB) at Assaf Harofeh (Shamir) Medical Center (Tel Aviv, Israel).

### Section 1.2: Immunological Testing

Immunoassays were performed using either the BD Veritor™ system for Rapid Detection of SARS-CoV-2 (Becton Dickinson and Company, Maryland, USA, Ref: 256082), or the COVID-19 Antigen Rapid Test Cassette (Beijing Kewei Clinical Diagnostic Reagent Inc., Beijing, China, Ref: 601450) in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs, and the COVID-19 Antigen Rapid Test Cassette is a solid phase immunochromatographic assay for the in vitro qualitative detection of SARS-CoV-2 antigen in nasopharyngeal, nasal, and throat swab specimens. Due to the limited availability of the BD Veritor™ system for Rapid Detection of SARS-CoV-2, in 4 subjects the anterior nares performed prior to nasal spraying and gargling was tested on the COVID-19 Antigen Rapid Test Cassette, with all other sample types tested on the BD Veritor™ system for Rapid Detection of SARS-CoV-2. In order to determine semi-quantitative differences in viral load between sample types, test band intensity was scored on a scale of 0-3, with 0 corresponding to a negative test.

### Section 2: Results and Discussion

7 subjects tested negative and 4 subjects tested positive for SARS-CoV-2 antigens on all sample types (anterior nares swabbing before nasal saline spraying and gargling, mid-turbinate swabbing, and anterior nares swabbing after nasal saline spraying and gargling).

6 out of the 9 subjects with discordant results (67%) tested positive for SARS-CoV-2 antigens both on the mid-turbinate swab and on the anterior nares swab performed after nasal saline spraying and gargling, but negative on the anterior nares swab performed before nasal saline spraying and gargling, indicating that nasal saline spraying increases anterior nares swab efficacy in viral material sampling and sensitivity with immunoassays. In a like manner, 1 out of the 9 subjects with discordant results tested clearly positive for SARS-CoV-2 antigens both on the mid-turbinate swab and on the anterior nares swab performed after nasal saline spraying and gargling; however, on the anterior nares swab prior to nasal saline spraying and gargling, it was unclear whether or not there was a faint band corresponding to the presence of SARS-CoV-2 antigens. Regarding the remaining 2 subjects with discordant results, 1 subject tested positive for SARS-CoV-2 antigens on the mid-turbinate swab, but negative on both anterior nares swabs, and 1 subject tested positive for SARS-CoV-2 antigens on the anterior nares swab performed after nasal saline spraying and gargling, but negative on both the mid-turbinate and anterior nares swab performed prior to nasal spraying and gargling. Similarly, positive test band intensity for SARS-CoV-2 antigens in anterior nares swab samples performed after nasal saline spraying and gargling was equal to that of the mid-turbinate swab samples for 2 out of the 15 subjects (13.3%) that presented a positive on both the anterior nares swab performed after nasal saline spraying and gargling and the mid-turbinate swab. Moreover, positive test band intensity for SARS-CoV-2 antigens was higher in anterior nares swab samples performed after nasal saline spraying and gargling than that of the mid-turbinate swab samples for 3 out of the 15 subjects (20%) that presented a positive on both the anterior nares swab performed after nasal saline spraying and gargling and the mid-turbinate swab, indicating that by coupling nasal spray with anterior nares swabbing, anterior nares swabs in some cases collect more viral material than mid-turbinate swabs.

In some applications, any of the techniques described in the report of Experiment 3 above are used in combination with the other techniques described herein. It is noted that although Experiment 3, as reported above, included mid-turbinate swabbing of the subjects, this mid-turbinate swabbing served for comparison with anterior nares swabbing. Some techniques of the present invention include performing only an anterior nares swab, without performing a mid-turbinate swab.

### Experiment 4

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using PCR. Experiment 4 provides evidence of the efficacy of these techniques for collecting specimen samples for detection of SARS-CoV-2 using quantitative PCR (qPCR).

### Detailed Report of Experiment 4: Benefits of Nasal Saline Spraying Before Anterior Nares Swabbing in SARS-CoV-2 Detection

### Section 1: Materials and Methods

### Section 1.1: Subject Sampling

Three bilateral anterior nares swab samplings were performed approximately one minute apart by a healthcare professional on 12 subjects belonging to the health care service provider Leumit (Tel-Aviv, Israel), who had previously tested positive for SARS-CoV-2. The subjects then sprayed 0.1 mL of normal saline 3-5 times into each nostril and gargled 10 mL of normal saline. Then a fourth bilateral anterior nares swab was performed by the healthcare professional. Bilateral anterior nares swabbing was performed by inserting swabs into the nares and then removing the swabs without rotating the swabs against the inner wall of the nares. Swabs were then placed into tubes containing 3 mL of Universal Transport Medium (UTM) (Biological Industries, Israel). Samples were transported on ice to the Leumit Health Services Central Laboratory (Or Yehuda, Israel), for processing. The clinical study was approved by the investigational review board (IRB) at Assaf Harofeh (Shamir) Medical Center (Tel Aviv, Israel). All swabbing was performed with flocked swabs (3BY Ltd., Industrial Park Tefen, Israel, Ref: 3by-001).

### Section 1.2: Molecular Testing

Samples were processed by qPCR at the Leumit Health Services Central Laboratory (Or Yehuda, Israel) using the Allplex 2019 n-CoV Assay (Seegene, Seoul, South Korea), following manufacturer's instructions, on a CFX96 Real-Time System (BIO RAD, USA). Prior to qPCR, samples were extracted using the STARMag 96×4 Viral DNA/RNA 200 C Kit (Seegene, Seoul, South Korea) on a MICROLAB STARlet (Hamilton, Switzerland) automated liquid handling platform. Statistical analysis was performed using the Wilcoxon signed rank test to determine p-values.

### Section 2: Results and Discussion

Lower Ct values were observed in anterior nares swabs performed after nasal saline spraying and gargling than before nasal saline spraying and gargling on average across all SARS-CoV-2 gene targets (Tables 7.1, 7.2), indicative of higher viral levels after nasal saline spraying and gargling than before nasal saline spraying and gargling independent of possible contributions from a mid-turbinate swab.

Average Ct values of the fourth anterior nares swab, which were preceded by nasal spraying and gargling, were lower across all gene targets than that of the other paired anterior nares swabs performed prior to nasal spraying and gargling (Table 7.2). The reversal of the observed aforementioned trend highlights the benefit of performing nasal spraying and gargling prior to anterior nares swabbing in SARS-CoV-2 detection, specifically in light of comparisons between the third anterior nares swab performed prior to nasal spraying and gargling and the fourth anterior nares swab performed following nasal spraying and gargling. The difference between the averages of the third nares swab performed prior to nasal spraying and gargling and the fourth anterior nares swab performed following nasal spraying and gargling reached statistical significance across all gene targets tested (Table 7.3) despite the relatively small sample size.

TABLE 7.1
Ct value results

| Subject | | C(t) values for each Gene Target^{ψ} | | | |
|---|---|---|---|---|---|
| # | Sampleⁱ | E | RdRP/S | N | ICⁱⁱ |
| 1 | AN1 | 40.0 | 40.0 | 40.0 | 25.6 |
| | AN2 | 33.7 | 36.0 | 36.0 | 27.1 |
| | AN3 | 40.0 | 40.0 | 40.0 | 27.4 |
| | AN4 (after NG) | 34.7 | 35.1 | 37.8 | 25.8 |
| 2 | AN1 | 25.6 | 27.5 | 37.8 | 26.7 |
| | AN2 | 28.8 | 31.5 | 27.8 | 26.9 |
| | AN3 | 26.2 | 28.7 | 31.1 | 25.7 |
| | AN4 (after NG) | 24.8 | 26.8 | 26.9 | 25.8 |
| 3 | AN1 | 25.0 | 26.9 | 27.2 | 25.7 |
| | AN2 | 24.3 | 26.1 | 26.5 | 25.4 |
| | AN3 | 23.0 | 25.0 | 24.4 | 24.8 |
| | AN4 (after NG) | 20.1 | 21.7 | 22.3 | 27.1 |
| 4 | AN1 | 40.0 | 40.0 | 40.0 | 40.0 |
| | AN2 | 40.0 | 40.0 | 40.0 | 40.0 |
| | AN3 | 40.0 | 40.0 | 40.0 | 40.0 |
| | AN4 (after NG) | 40.0 | 40.0 | 40.0 | 40.0 |
| 5 | AN1 | 36.4 | 40.0 | 39.5 | 26.7 |
| | AN2 | 40.0 | 40.0 | 36.9 | 25.5 |
| | AN3 | 40.0 | 40.0 | 40.0 | 26.7 |
| | AN4 (after NG) | 40.0 | 40.0 | 40.0 | 25.2 |
| 6 | AN1 | 28.4 | 30.5 | 31.3 | 26.7 |
| | AN2 | 29.2 | 30.9 | 31.7 | 25.5 |
| | AN3 | 30.4 | 32.6 | 33.0 | 26.7 |
| | AN4 (after NG) | 28.0 | 29.4 | 30.4 | 25.2 |
| 7 | AN1 | 40.0 | 40.0 | 37.9 | 25.7 |
| | AN2 | 40.0 | 36.7 | 40.0 | 25.6 |
| | AN3 | 40.0 | 40.0 | 40.0 | 26.9 |
| | AN4 (after NG) | 40.0 | 40.0 | 40.0 | 27.1 |
| 8 | AN1 | 40.0 | 40.0 | 40.0 | 25.6 |
| | AN2 | 40.0 | 40.0 | 40.0 | 27.1 |
| | AN3 | 40.0 | 40.0 | 40.0 | 26.9 |
| | AN4 (after NG) | 40.0 | 37.1 | 40.0 | 25.4 |
| 9 | AN1 | 40.0 | 40.0 | 40.0 | 27.2 |
| | AN2 | 40.0 | 40.0 | 40.0 | 27.3 |
| | AN3 | 40.0 | 40.0 | 40.0 | 27.1 |
| | AN4 (after NG) | 40.0 | 39.2 | 40.0 | 27.3 |
| 10 | AN1 | 40.0 | 40.0 | 40.0 | 40.0 |
| | AN2 | 40.0 | 40.0 | 40.0 | 40.0 |
| | AN3 | 40.0 | 40.0 | 40.0 | 40.0 |
| | AN4 (after NG) | 40.0 | 40.0 | 40.0 | 40.0 |
| 11 | AN1 | 32.3 | 34.2 | 33.7 | 25.8 |
| | AN2 | 32.8 | 34.5 | 33.7 | 25.5 |
| | AN3 | 31.9 | 33.3 | 34.4 | 26.9 |
| | AN4 (after NG) | 28.3 | 29.6 | 30.3 | 26.0 |
| 12 | AN1 | 24.8 | 26.6 | 26.7 | 25.4 |
| | AN2 | 29.6 | 31.1 | 31.6 | 25.5 |
| | AN3 | 26.4 | 28.0 | 28.8 | 25.7 |
| | AN4 (after NG) | 25.9 | 27.8 | 28.3 | 25.8 |

ⁱAN1, AN2, AN3 = Three anterior nares swabs performed in succession prior to nasal spraying and gargling (NG); AN4 = Anterior nares swab performed after NG
ⁱⁱIC = Internal control used in molecular assay
^{ψ}Negative Ct values are expressed as 40

TABLE 7.2
Ct value averagesⁱⁱⁱ

| | C(t) value averages for each Gene Target | | |
|---|---|---|---|
| Sample | E | RdRP/S | N |
| AN1 | 31.56 | 33.21 | 34.27 |
| AN2 | 32.30 | 33.36 | 33.04 |
| AN3 | 32.24 | 33.45 | 33.95 |
| AN4 (after NG) | 30.21 | 31.29 | 31.99 |

ⁱⁱⁱFor the purpose of making a meaningful comparison between swab samples, average Ct values exclude subjects with less than two positive gene targets across all samples (excluded: subject 4, subject 8, subject 9, and subject 10)

TABLE 7.3
Statistical Significance: p-values^{iv}

| | p-values for each Gene Target | | |
|---|---|---|---|
| Paired Samples | E | RdRP/S | N |
| AN1—AN2 | 0.447 | 0.866 | 0.554 |
| AN1—AN3 | 0.344 | 0.590 | 0.933 |
| AN1—AN4 | 0.272 | 0/142 | 0.195 |
| AN2—AN3 | 0.675 | 0/800 | 0.272 |
| AN2—AN4 | 0.059 | 0.076 | 0.272 |
| AN3—AN4 | 0.036* | 0.036* | 0.036* |

^{iv}The Wilcoxon signed rank test was used to determine p-values. For the purpose of making a meaningful comparison between swab samples, statistical tests excluded subjects with less than two positive gene targets across all samples (excluded: subject 4, subject 8, subject 9, and subject 10)
*Indicates statistically significant values

In some applications, any of the techniques described in the report of Experiment 4 above are used in combination with the other techniques described herein.

### Experiment 5

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay. Experiment 5 provides evidence of the efficacy of these techniques for collecting specimen samples for detection of SARS-CoV-2 using lateral flow immunoassay strips.

In summary, Experiment 5 found that, in a single subject, the SARS-CoV-2 antigen test band intensity for an anterior nares swab performed following nasal saline spraying was greater than that of both an anterior nares swab performed prior to nasal saline spraying and a mid-turbinate swab, indicating a greater quantity of virus recovered on an anterior nares swabs performed following nasal saline spraying than on anterior nares swab performed prior to nasal saline spraying and a mid-turbinate swab.

### Detailed Report of Experiment 5: Further Evaluation of the Benefits of Nasal Saline Spraying without Gargling Prior to Anterior Nares Swabbing in SARS-CoV-2 Detection by Immunoassay

### Section 1: Materials and Methods

### Section 1.1: Subject Sampling

Bilateral anterior nares swabbing was performed by a healthcare professional on 7 subjects belonging to the health care service provider Leumit (Tel-Aviv, Israel), who had previously tested positive for SARS-CoV-2. The subjects then sprayed 0.1 mL of normal saline 3-5 times into each nostril with their heads in a neutral position or slightly tilted back. Subjects then significantly tilted their heads backwards by extending their necks as far as comfortably possible and maintained this head positioning for 5-10 seconds, similar to the conditions that normally occur during gargling. The subjects then returned their heads to a neutral position and a second bilateral anterior nares swabbing was performed by the healthcare professional. Lastly, bilateral mid-turbinate swabbing was performed by the healthcare professional. Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001). The clinical study was approved by the investigational review board (IRB) at Assaf Harofeh (Shamir) Medical Center (Tel Aviv, Israel).

### Section 1.2: Immunological Testing

Immunoassays were performed on all sample types using the BD Veritor™ system for Rapid Detection of SARS-CoV-2 (Becton Dickinson and Company, Maryland, USA, Ref: 256082), in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs. In order to determine semi-quantitative differences in viral load between sample types, test band intensity was scored on a scale of 0-3, with 0 corresponding to a negative test.

### Section 2: Results and Discussion

Six of the seven subject samples were close to their 10-day quarantine release date, or had already been released, and unsurprisingly tested negative on all immunoassays performed. The single remaining subject who had tested positive for SARS-CoV-2 by qPCR 4 days prior to sample collection tested positive for the presence of SARS-CoV-2 antigen on all sample types (anterior nares swab prior to nasal saline spraying, anterior nares swab following nasal saline spraying, and the mid-turbinate swab) by immunoassay. The SARS-CoV-2 antigen test band intensity for the anterior nares swab performed following nasal saline spraying (score: 3) was greater than that of both the anterior nares swab performed prior to nasal saline spraying (score: 2) and the mid-turbinate swab (score: 2.5), indicating a greater quantity of virus recovered on an anterior nares swab performed following nasal saline spraying than on an anterior nares swab performed prior to nasal saline spraying and a mid-turbinate swab.

This observation supports the fact that coupling nasal saline spraying to anterior nares swabbing increases immunoassay testing sensitivity. As subjects did not gargle saline as in the previous experiments described hereinabove in Experiments 1, 2, 3, and 4, it appears that saline spraying alone is sufficient for increasing anterior nares testing sensitivity.

In some applications, any of the techniques described in the report of Experiment 5 above are used in combination with the other techniques described herein.

### Experiment 6

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using an immunoassay or using isothermal amplification. Experiment 6 provides evidence of the efficacy of these techniques for collecting specimen samples for detection of SARS-CoV-2 using lateral flow immunoassay strips and using isothermal amplification.

In summary, Experiment 6 found that the sensitivity of lateral flow immunoassay strips improve for anterior nares swabs performed following nasal saline spraying compared to anterior nares swabs performed prior to nasal saline spraying and that, on average, the BARS-CoV-2 antigen test band intensity for anterior nares swabs performed following nasal saline spraying were greater than that of anterior nares swabs performed prior to nasal saline spraying, indicating a greater quantity of virus recovered on anterior nares swabs performed following nasal saline spraying than on anterior nares swab performed prior to nasal saline spraying, in support of Experiment 5 hereinabove. Experiment 6 additionally found that the sensitivity of isothermal amplification SARS-CoV-2 detection tests also improve for anterior nares swabs performed following nasal saline spraying compared to anterior nares swabs performed prior to nasal saline spraying.

Detailed Description of Experiment 6: Further Evaluation of Nasal Saline Spraying without Gargling Prior to Anterior Nares Swabbing in SARS-CoV-2 Detection by Immunoassay and Isothermal Amplification and Effects of Head Positioning

### Section 1: Materials and Methods

### Section 1.1: Patient Population

This experiment was performed by collecting samples from 14 subjects belonging to the health care service provider Leumit (Tel-Aviv, Israel), who had previously tested positive for SARS-CoV-2. Of the 14 subjects, 6 subjects were subsequently retested the following day (n=20). The clinical study was approved by the investigational review board (IRB) at Assaf Harofeh (Shamir) Medical Center (Tel Aviv, Israel).

### Section 1.2: Subject Sampling

Two alternative sample collection procedures were implemented during subject sampling. First, a unilateral anterior naris swabbing of the right nostril was performed by a healthcare professional. Subjects then sprayed 0.1 mL of normal saline 3-5 times into the right nostril with their heads in a neutral position or slightly tilted back, sniffed in deeply, and then tilted their heads backwards by extending their necks as far as comfortably possible and maintained this head positioning for approximately 15 seconds. The subjects then returned their heads to a neutral position and a second unilateral anterior naris swabbing of the right nostril and then a mid-turbinate swabbing of the right nostril were performed by a healthcare professional. A unilateral anterior naris swabbing of the left nostril was then performed by a healthcare professional. As with the right nostril, subjects then sprayed 0.1 mL of normal saline 3-5 times into the left nostril with their heads in a neutral position or slightly tilted back. Subjects then maintained the neutral or slightly tilted back head positioning for approximately 10 seconds. Lastly, a second unilateral anterior naris swabbing of the left nostril was performed by a healthcare professional.

Alternatively, following the unilateral anterior naris swabbing of the right nostril and then nasal saline spraying into the right nostril as described above, subjects maintained the neutral or slightly tilted back head positioning for approximately 10 seconds followed by a second unilateral anterior naris swabbing and then a mid-turbinate swabbing of the right nostril by the healthcare professional. Following the unilateral anterior naris swabbing of the left nostril and then nasal saline spraying into the left nostril as described above, subjects then tilted their heads backwards by extending their necks as far as comfortably possible and maintained this head positioning for approximately 15 seconds. The subjects then returned their heads to a neutral position and a second unilateral anterior naris swabbing of the left nostril was performed by a healthcare professional.

Six subjects were resampled the following day such that the head positioning maintained following nasal saline spraying was reversed for each nostril during resampling compared to during original sampling (Table 8.1).

Dry swab samples were transported in 15 mL conical tubes (LabCon, USA, Ref #: LC 3136-345-008-9) on ice to Hero Scientific Ltd. (Jerusalem, Israel) for processing. All swabbing was performed with flocked swabs (3BY Ltd., Industrial Park Tefen, Ref #: 3by-001).

### Section 1.3: Testing

Most swab samples were tested by lateral flow immunoassay strips, except for four swab samples: two pairs of unilateral outer naris swab samples that were collected from the left nostril of two subjects with different head positionings were tested by isothermal amplification instead of by lateral flow immunoassay strips (Table 8.1).

Immunoassays were performed using the BD Veritor™ system for Rapid Detection of SARS-CoV-2 (Becton Dickinson and Company, Maryland, USA, Ref: 256082), in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs. In order to determine semi-quantitative differences in viral load between sample types, test band intensity was scored on a scale of 0-3, with 0 corresponding to a negative test.

Isothermal amplification tests were performed using the Lucira™ COVID-19 Ali-In-One Test Kit system for Rapid Detection of SARS-CoV-2 (Lucira Health, Emeryville, Calif.), in accordance with manufacturer instructions. The Lucira™ COVID-19 All-In-One Test Kit system for Rapid Detection of SARS-CoV-2 is isothermal amplification detection test that utilizes RT-LAMP technology to detect RNA of the N gene for SARS-CoV-2. A positive qualitative result is reported if an RNA signal is detected within 30 minutes and a negative qualitative result is reported if an RNA signal is not detected within 30 minutes. In order to determine semi-qualitative differences in viral load between sample types, the inventors considered the amount of time until results displayed as a relative indicator, with relatively less time until results displayed as an indicator of greater viral load and relatively more time until results displayed as an indicator of lower viral load.

TABLE 8.1
Overview of Head Positioning Durins Subject Samplins.
Detection Test Used, and Procedural Deviations

| | | Head | Detection Test |
|---|---|---|---|
| Subject | Nostril | Positionⁱ | Used |
| 1 | Right | Neutral | Immunoassay |
| | Left | Back | Immunoassay |
| 2 | Right | Back | Immunoassay |
| | Left | Neutral | Immunoassay |
| 3 | Right | Neutral | Immunoassay |
| | Left | Back | Immunoassay |
| 4 | Right | Back | Immunoassay |
| | Left | Neutral | Immunoassay |
| 5 | Right | Neutral | Immunoassay |
| | Left | Back | Immunoassay |
| 6 | Right | Back | Immunoassay |
| | Left | Neutral | Immunoassay |
| 7^{§} | Right | Neutral | Immunoassay |
| | Left | Back | Immunoassay |
| 8^{§} | Right | Back | Immunoassay |
| | Left | Neutral | Immunoassay |
| 9 | Right | Neutral | Immunoassay |
| | Left | Back | Immunoassay |
| 10 | Right | Back | Immunoassay |
| | Left | Neutral | Immunoassay |
| 11 | Right | Neutral | Immunoassay |
| | Left | Back | Immunoassay |
| 12 | Right | Back | Immunoassay |
| | Left | Neutral | Immunoassay |
| 13^{Ω} | Right | Back | Immunoassay |
| | Left | Neutral | Immunoassay |
| 14 | Right | Neutral | Immunoassay |
| | Left | Back | Immunoassay |
| 4* | Right | Neutral | Immunoassay |
| | Left | Back | Immunoassay |
| 5* | Right | Back | Immunoassay |
| | Left | Neutral | Isothermal |
| | | | Amplification |
| 6* | Right | Neutral | Immunoassay |
| | Left | Back | Isothermal |
| | | | Amplification |
| 7* | Right | Back | Immunoassay |
| | Left | Neutral | Immunoassay |
| 9* | Right | Back | Immunoassay |
| | Left | Neutral | Immunoassay |
| 11* | Right | Back | Immunoassay |
| | Left | Neutral | Immunoassay |

ⁱHead positioning immediately following nasal saline spraying. “Back” indicates that the subject maintained a completely tilted back head position for approximately 15 seconds, “Neutral” indicated that the subject maintained a neutral or slightly tilted of back head position for approximately 10 seconds.
^{Ω}Subject did not sniff in after nasal saline spraying.
*Resampling subjects with switched head positioning.
^{§}Immunoassay test band intensity corresponding to SARS-CoV-2 antigen presence was not scored

### Section 2: Results and Discussion

Immunoassay results were obtained from a total of 38 paired unilateral anterior naris swabs collected before and after nasal saline spraying: 19 paired unilateral anterior naris swabs for each head position (neutral or slightly tilted, and completely tilted back). Isothermal amplification results were obtained from a total of 2 paired unilateral anterior naris swabs collected before and after nasal saline spraying: 1 paired unilateral anterior naris swab for each head position (neutral or slightly tilted, and completely tilted back). Because of expected differences in viral loads between nostrils caused by the nasal cycle, internal comparisons between sampling methodology by pairing nostrils on a patient-by-patient basis were not made, but rather samples from each nostril were treated as samples from independent subjects. Similarly, conclusions were not drawn from comparisons between original samples produced from subjects and samples collected after resampling with alternate methodologies due to temporal changes in nostril dominance, but rather were treated as samples from independent subjects.

### Section 2.1: Immunoassay Sensitivity Data

11 out of the 19 paired unilateral anterior naris swabs collected from subjects following the sampling protocol in which the head was completely tilted back after nasal saline spraying (hereinbelow, “Head Tilt Protocol”), and 9 out of the 19 paired unilateral anterior naris swabs collected from subjects following the sampling protocol in which the head was neutral or slightly tilted after nasal saline spraying (hereinbelow, “Head Neutral Protocol”), were negative for SARS-CoV-2 antigens on both unilateral anterior naris swabs collected before and after nasal saline spraying. 5 out of the remaining 8 paired unilateral anterior naris swabs collected from subjects following the Head Tilt Protocol, and 4 out of the remaining 10 paired unilateral anterior naris swabs collected from subjects following the Head Neutral Protocol, tested positive for both unilateral anterior naris swabs collected before and after nasal saline spraying.

Discordant results were reported for 3 paired unilateral anterior naris swabs collected before and after nasal saline spraying from subjects following the Head Tilt Protocol. For all 3 discordant swabs, the unilateral anterior naris swab samples taken after nasal saline spraying were positive for SARS-CoV-2 antigen, and the unilateral anterior naris swab samples taken before nasal saline spraying were negative for SARS-CoV-2 antigen. As seen in Table 8.2, following the Head Tilt Protocol, unilateral anterior naris swabs taken before nasal saline spraying were observed to be 62.5% sensitivity (5/8), while unilateral anterior naris swabs taken after nasal saline spraying had 100% sensitivity (8/8), indicating a 60% increase in detection sensitivity associated with unilateral anterior naris swabbing after nasal saline spraying following the Head Tilt Protocol relative to unilateral anterior naris swabbing performed prior to nasal saline spraying.

Discordant results were reported for 6 paired unilateral anterior naris swabs collected before and after nasal saline spraying from subjects following the Head Neutral Protocol. For 5 of the 6 discordant results, the unilateral anterior naris swab samples taken after nasal saline spraying was positive for SARS-CoV-2 antigen, and the unilateral anterior naris swab samples taken before nasal saline spraying were negative for SARS-CoV-2 antigen. Conversely, for the single discordant result following the Head Neutral Protocol, the unilateral anterior naris swab sample taken after nasal saline spraying was negative for SARS-CoV-2 antigen, and the unilateral anterior naris swab sample taken prior to nasal saline spraying was positive for SARS-CoV-2 antigen. As seen in Table 8.2, following the Head Neutral Protocol, unilateral anterior naris swabs taken before nasal saline spraying were observed to be 50% sensitivity (5/10), while unilateral anterior naris swabs taken after nasal saline spraying had 90% sensitivity (9/10), indicating an 80% increase in detection sensitivity associated with unilateral anterior naris swabbing after nasal saline spraying following the Head Neutral Protocol relative to unilateral anterior naris swabbing performed prior to nasal saline spraying.

Because nasal saline spraying increased the sensitivity of the unilateral anterior naris swabs sampled after nasal saline spraying compared to the paired unilateral anterior naris swabs sampled before nasal saline spraying in both Head Neutral and Head Tilt Protocols, it appears that the nasal saline spraying can increase the presence of SARS-CoV-2 virus in the anterior nares independent of head positioning. Furthermore, these observations support previous findings reported in Experiment 5, described hereinabove, that nasal saline spraying benefits SARS-CoV-2 detection on anterior nares swabs in a gargle-independent manner. Results reported here appear to support the validity of the Head Neutral Protocol over that of the Head Tilt Protocol. However, given that unilateral anterior naris swabs sampled prior to nasal saline spraying were 25% more sensitive for those sampled during testing of the Head Tilt Protocol relative to those sampled during testing of the Head Neutral Protocol (62.5% and 50%, respectively), despite differences between protocols only effecting samples taken after nasal saline spraying (unilateral anterior naris swabbing after nasal saline spraying), it is likely that relative differences between protocols are at least partially due to the small sample size. Moreover, and more importantly, as unilateral anterior naris swabbing was performed immediately after returning the head to a normal or slightly tilted back position in the Head Tilt Protocol, it is possible that saline which sampled the virus-rich nasopharyngeal regions had not had sufficient time to drip back into the anterior nares for sampling, as opposed to the Head Neutral Protocol, where the head positioning was maintained for 10 seconds and was thus conducive to saline drippage.

TABLE 8.2
Immunoassay Sensitivity

| | Head Neutral Protocol | | | Head Tilt Protocol | | |
|---|---|---|---|---|---|---|
| Subject | Nostril | AN1 | AN2 | Nostril | AN1 | AN2 |
| 1 | Right | 0 | 0 | Left | 0 | 0 |
| 2 | Left | 0 | 0 | Right | 0 | 0 |
| 3 | Right | 0 | 0 | Left | 0 | 0 |
| 4 | Left | 0 | 1 | Right | 0 | 0 |
| 5 | Right | 0 | 0 | Left | 0 | 0 |
| 6 | Left | 0 | 0 | Right | 0 | 0 |
| 7 | Right | 0 | 1 | Left | 0 | 1 |
| 8 | Left | 1 | 0 | Right | 0 | 0 |
| 9 | Right | 0 | 1 | Left | 1 | 1 |
| 10 | Left | 0 | 0 | Right | 0 | 0 |
| 11 | Right | 0 | 0 | Left | 1 | 1 |
| 12 | Left | 1 | 1 | Right | 0 | 0 |
| 13 | Left | 1 | 1 | Right | 1 | 1 |
| 14 | Right | 0 | 1 | Left | 0 | 0 |
| 4* | Right | 0 | 0 | Left | 1 | 1 |
| 5* | Left | NA | NA | Right | 0 | 0 |
| 6* | Right | 0 | 0 | Left | NA | NA |
| 7* | Left | 0 | 1 | Right | 0 | 1 |
| 9* | Left | 1 | 1 | Right | 1 | 1 |
| 11* | Left | 1 | 1 | Right | 0 | 1 |
| Sensitivity | | 50% | 90% | | 62.5% | 100% |
| | | (5/10) | (9/10) | | (5/8) | (8/8) |

i-Samples were assumed true positive if at least one of the paired unilateral anterior naris swabs before and/or after nasal saline spraying was positive for SARS-CoV-2 antigen. Samples taken from different nostrils from the same subject were assessed as separate subjects.
*-Resampling subjects with switched head positioning.

### Section 2.2: Immunoassay Test Band Intensity Data

With regards to the Head Tilt Protocol, for the 5 subjects for whom both of the paired unilateral anterior naris swabs were positive for SARS-CoV-2 antigens, 60% of the time (3/5) test band intensity was greater for unilateral anterior naris swabs sampled after nasal saline spraying than unilateral anterior naris swabs sampled before nasal saline spraying. Similarly, for the 4 subjects for whom both of the paired unilateral anterior naris swabs were positive for SARS-CoV-2 antigens following the Head Normal Protocol, 75% of the time (3/4) test band intensity was greater for unilateral anterior naris swabs sampled after nasal saline spraying than unilateral anterior naris swabs sampled before nasal saline spraying, with equal test band intensities for both paired unilateral anterior naris swabs for the remaining subject. The overall trend of stronger test band intensities associated with unilateral anterior naris swabs sampled after nasal saline spraying, indicating sampling of more viral material further demonstrates the efficacy of incorporating nasal saline spraying into anterior nares swab sampling methods independent of head positioning.

### Section 2.3: Isothermal Amplification Sensitivity Data

As seen in Table 8.3, one of the two paired unilateral anterior naris swabs collected from subjects following either the Head Tilt Protocol or Head Neutral Protocol tested positive for both unilateral anterior naris swabs collected before and after nasal saline spraying. Discordant results were reported for the one remaining paired unilateral anterior naris swabs collected before and after nasal saline spraying. For this one pair of discordant swabs, the unilateral anterior naris swab sample taken after nasal saline spraying was positive for SARS-CoV-2 antigen, and the unilateral anterior naris swab sample taken before nasal saline spraying was negative for SARS-CoV-2 antigen.

Furthermore, the one pair of unilateral anterior naris swabs with non-discordant results showed a difference in the amount of time until the isothermal amplification detection test displayed results. The tested unilateral anterior naris swab sample taken before nasal saline spraying displayed results after 19 minutes, while the tested unilateral anterior naris swab sample taken after nasal saline spraying displayed results after only 15 minutes, indicating that the unilateral anterior naris swab sample taken after nasal saline spraying had a relatively greater viral load compared to the unilateral anterior naris swab sample taken before nasal saline spraying.

TABLE 8.3
Isothermal Amplification Results

| | | Head | | | Time Until | |
|---|---|---|---|---|---|---|
| Subject | Nostril | Position | Swab | Result | Displayed (min) | Result |
| 5* | Left | Neutral | AN1 | 0 | 30 (maximum) | |
| | | | AN2 | 1 | 22 | |
| 6* | Left | Back (Tilt) | AN1 | 1 | 19 | |
| | | | AN2 | 1 | 15 | |

### Section 3: Concluding Remarks

The evidence presented herein shows that the sampling methods described herein that comprise saline nasal administration (e.g., by spraying) prior to anterior nares swabbing not only increase immunoassay sensitivity, but also antigen test band intensities compared to anterior nares swabbing alone, which, for example, may help in examination of immunoassay results even without the aid of an automated immunoassay reader.

Additionally, the evidence presented herein shows that the sampling methods described herein that comprise saline nasal administration (e.g., by spraying) prior to anterior nares swabbing increase isothermal amplification testing sensitivity and decrease the amount of time until positive results are displayed. As in Experiment 5, subjects did not gargle saline as in Experiments 1, 2, 3, and 4 described hereinabove, indicating that saline spraying alone is sufficient for increasing anterior nares testing sensitivity. This data also suggest the benefit of saline nasal administration prior to anterior nares swabbing independent of head positioning, since sensitivity increased when using a nasal saline spraying protocol involving a neutral head positioning and when using a nasal saline spraying protocol involving a tilted head positioning. However, the evidence presented herein is insufficient for determining which head positioning protocol is superior, since results were likely confounded by procedural variations between examined protocols (i.e., unilateral anterior naris swabbing was performed immediately after returning the head to a normal or slightly tilted back position in the Head Tilt Protocol, so it is possible that saline which sampled the virus-rich nasopharyngeal regions had not had sufficient time to drip back into the anterior nares for sampling, as opposed to the Head Neutral Protocol, where the head positioning was maintained for 10 seconds and was thus conducive to saline drippage).

In some applications, any of the techniques described in the report of Experiment 6 above are used in combination with the other techniques described herein.

### Experiment 7

Some of the techniques described hereinabove comprise intranasally dispensing nasal wash fluid into a nasal cavity of a subject; and thereafter, collecting a specimen sample by performing an anterior nares swab, without performing a mid-turbinate swab or a nasopharyngeal swab. Optionally, the method further comprises testing for the presence of a particulate, such as a virus (e.g., SARS-CoV-2), for example using quantitative PCR (qPCR). Experiment 7 provides evidence of the efficacy of these techniques for collecting specimen samples for detection of SARS-CoV-2 using qPCR.

In summary, Experiment 7 found that detection test sensitivity increases when the subject tilts his or her head as far back as comfortably possible after saline nasal administration prior to anterior nares swabbing compared to when saline nasal administration is performed with a head neutral positioning prior to anterior nares swabbing. Experiment 7, unlike Experiment 6, included a wait time between returning the head to a neutral or slightly tilted back position and performing an anterior nares swab sampling, which improved viral load in the sample and improved test sensitivity.

### Detailed Report of Experiment 7: Effects of Head Positioning on Nasal Saline Spraying without Gargling Prior to Anterior Nares Swabbing in SARS-CoV-2 Detection by qPCR

### Section 1: Materials and Methods

### Section 1.1: Patient Population

This experiment was performed by collecting samples from 4 subjects belonging to the health care service provider Leumit (Tel-Aviv, Israel), who had previously tested positive for SARS-CoV-2. The clinical study was approved by the investigational review board (IRB) at Assaf Harofeh (Shamir) Medical Center (Tel Aviv, Israel).

### Section 1.2: Subject Sampling

A unilateral anterior naris swabbing of the right nostril was performed by a healthcare professional. Subjects then sprayed 0.1 mL of normal saline 3-5 times into the right nostril with their heads in a neutral position or slightly tilted back, sniffed in deeply, and then tilted their heads backwards by extending their necks as far as comfortably possible and maintained this head positioning for approximately 15 seconds. The subjects then returned their heads to a neutral or slightly tilted back position and waited approximately 10 seconds before a second unilateral anterior naris swabbing of the right nostril was performed by the healthcare professional. A mid-turbinate swabbing of the right nostril was then performed by a healthcare professional. A unilateral anterior naris swabbing of the left nostril was then performed by a healthcare professional. As with the right nostril, subjects then sprayed 0.1 mL of normal saline 3-5 times into the left nostril with their heads in a neutral position or slightly tilted back. Subjects then maintained the neutral or slightly tilted back head positioning for approximately 10 seconds. Lastly, a second unilateral anterior naris swabbing of the left nostril was performed by a healthcare professional. A single subject felt unwell and refused the final unilateral anterior nares swabbing (left nostril, after nasal saline spraying).

All swabs were placed into tubes containing 3 mL of Universal Transport Medium (UTM) (Biological Industries, Israel) and transported on ice to the Leumit Health Services Central Laboratory (Or Yehuda, Israel), for processing via qPCR.

### Section 1.3: Molecular Testing

RNA extraction and qPCR were performed using the Cobas SARS-CoV-2 Test (Roche, Switzerland) on a Cobas 6800 system (Roche, Switzerland), in accordance with manufacturer instructions.

### Section 2: Results and Discussion

Average Ct score difference between unilateral anterior nares swabs collected before nasal saline spraying and unilateral anterior nares swabs collected after nasal saline spraying were greater when subjects tilted their heads back (“Head Tilting Protocol”) after nasal saline spraying compared to when subjects maintained a neutral or slightly tilted back head position (“Head Neutral Protocol”) after nasal saline spraying (Table 9.3).

TABLE 9.1
c/PCR resultsⁱⁱ

| | | Ct Scores | |
|---|---|---|---|
| Subject | Sampleⁱ | ORF1a/b | E |
| 1 | Tilt-AN1 | 36.45 | 38.08 |
| | Tilt-AN2 | 36.67 | 40 |
| | Neutral-AN1 | 31.98 | 33.32 |
| | Neutral-AN2 | 31.46 | 32.67 |
| 2 | Tilt-AN1 | 35.16 | 36.83 |
| | Tilt-AN2 | 29.66 | 30.36 |
| | Neutral-AN1 | 29.31 | 29.75 |
| | Neutral-AN2ⁱⁱⁱ | N/A | N/A |
| 3 | Tilt-AN1 | 33.0 | 34.42 |
| | Tilt-AN2 | 31.53 | 32.38 |
| | Neutral-AN1 | 33.27 | 34.77 |
| | Neutral-AN2 | 33.6 | 35.19 |
| 4 | Tilt-AN1 | 27.86 | 28.36 |
| | Tilt-AN2 | 22.85 | 23.24 |
| | Neutral-AN1 | 27.31 | 27.8 |
| | Neutral-AN2 | 24.59 | 24.89 |

ⁱ“Tilt”—swabbing was performed on right nostril, wherein the head was tilted after nasal saline spraying. “Neutral”—swabbing was performed on the left nostril, wherein the head remained in a neutral or slightly tilted back position following nasal saline spraying. “AN1”—Unilateral anterior nares swabbing performed prior to nasal saline spraying. “AN2”—Unilateral anterior nares swabbing performed after nasal saline spraying.
ⁱⁱNegative PCR results are represented by a Ct score of 40.
ⁱⁱⁱSubject refused sampling.

TABLE 9.2
Ct Score Differences

| | Head | Ct Score Differenceⁱ | |
|---|---|---|---|
| Subject | Positionⁱⁱ | ORF1a/b gene | E gene |
| 1 | Tilt | 0.22 | 1.92 |
| | Neutral | −0.52 | −0.65 |
| 2 | Tilt | −5.5 | −6.47 |
| | Neutral | N/A | N/A |
| 3 | Tilt | −1.47 | −2.04 |
| | Neutral | 0.33 | 0.42 |
| 4 | Tilt | −5.01 | −5.12 |
| | Neutral | −2.72 | −2.91 |

ⁱCt score differences were calculated by subtracting Ct scores of unilateral anterior nares swabs performed prior to nasal saline spraying from Ct scores of paired unilateral anterior nares swabbing performed after nasal saline spraying.
ⁱⁱ“Tilt”—swabbing was performed on right nostril, wherein the head was tilted after nasal saline spraying. “Neutral”—swabbing was performed on the left nostril, wherein the head remained in a neutral or slightly tilted back position following nasal saline spraying.

TABLE 9.3
Average Ct Score Difference

| Head | Average Ct Score Difference | |
|---|---|---|
| Positionⁱ | ORF1a/b gene | E gene |
| Tilt | −2.94 | −2.93 |
| Neutral | −0.97 | −1.04 |

ⁱ“Tilt”—swabbing was performed on right nostril, wherein the head was tilted after nasal saline spraying. “Neutral”—swabbing was performed on the left nostril, wherein the head remained in a neutral or slightly tilted back position following nasal saline spraying.

### Section 3: Concluding Remarks

Although Experiment 6, described hereinabove, may indicate that the Head Tilting Protocol is inferior to the Head Neutral Protocol for virus sampling, it is likely that the addition of a wait period before subjects return their heads to a neutral or slightly tilted position from the tilted back position, and/or the addition of a wait time after the subjects returned their heads to the neutral or slightly tilted back position, before swabbing (which was not included in the sampling protocol for Experiment 6 but was included in the sampling protocol for this Experiment 7) facilitates the dripping of saline from within the nasal cavity into the anterior nares. It therefore appears that a combination of the Head Tilting Protocol and a wait time before subjects return their heads to a neutral or slightly tilted back position, and/or a wait time after the subjects return their heads to the neutral or slightly tilted back position, and performing an anterior nares swab sampling improves viral load in the sample and improves test sensitivity.

In some applications, any of the techniques described in the report of Experiment 7 above are used in combination with the other techniques described herein.

### Experiment 8

Some of the techniques described hereinabove comprise intranasally dispensing nasal wash fluid into a nasal cavity of a subject; thereafter, collecting a specimen sample by performing an anterior nares swab, without performing a mid-turbinate swab or a nasopharyngeal swab; and testing for the presence of a particulate, such as a virus (e.g., SARS-CoV-2), using an immunoassay. Experiment 8 provides evidence of the efficacy of these techniques for collecting specimen samples for detection of SARS-CoV-2 using lateral flow immunoassay strips.

In summary, Experiment 8 compared qualitative and semi-quantitative immunoassay results using three sampling techniques: mid-turbinate swabbing, anterior nares swabbing before nasal saline spraying, and anterior nares swabbing after nasal saline spraying.

Experiment 8 found that 64% of subjects with discordant results tested positive for SARS-CoV-2 antigens both with mid-turbinate swabbing and anterior nares swabbing after nasal saline spraying, but negative with anterior nares swabbing before nasal saline spraying, indicating that nasal saline spraying increases anterior nares swab efficacy in viral material sampling and sensitivity in immunoassays.

Experiment 8 also found that test band intensity for SARS-CoV-2 antigens was higher for anterior nares swabbing after nasal saline spraying than for anterior nares swabbing before nasal saline spraying in 89% of the concordant positive results (i.e., concordant for all sample types), and equal for the remaining single concordant positive result (11%). In examining the relative immunoassay intensities of the two types of anterior nares swabbing (before and after nasal saline spraying) in subjects with both discordant and concordant results, 85% of subjects had stronger SARS-CoV-2 test band intensities from the anterior nares swab performed after nasal saline spraying compared to the paired anterior flares swab performed before nasal saline spraying.

### Benefits of Nasal Saline Spraying Prior to Anterior Nares Swabbing in SARS-CoV-2 Detection by Immunoassay

### Section 1: Materials and Methods

### Section 1.1: Subject Sampling

Bilateral anterior nares swabbing was performed by a healthcare professional on 44 subjects belonging to the health care service provider Leumit (Tel-Aviv, Israel), who had previously tested positive for SARS-CoV-2 by PCR. The subjects then sprayed 0.1 mL of normal saline 3-5 times into each nostril, and then some patients gargled 10 mL of normal saline for 10-15 seconds to produce a Nasal Gargle specimen. A few seconds after intranasally spraying normal saline or following spitting out of the Nasal Gargle sample, a second bilateral anterior nares swab was performed by the healthcare professional. A bilateral mid-turbinate swab was performed by the healthcare professional either after the first bilateral anterior nares swabbing or after the second bilateral anterior nares swabbing.

Dry swab samples were transported in 15 mL conical tubes (LabCon, USA, Ref #: LC 3136-345-008-9) on ice to Hero Scientific Ltd. (Jerusalem, Israel) for processing. All swabbing was performed with either flocked swabs (Biological Industries, Israel, Ref #: 3BY001) or swabs with polyurethane foam tips (Puritan, Maine, USA, Ref: 25-1506-1PF 100). The clinical study was approved by the investigational review board (IRB) at Assaf Harofeh (Shamir) Medical Center (Tel Aviv, Israel).

### Section 1.2: Immunological Testing

Immunoassays were performed using the BD Veritor™ system for Rapid Detection of SARS-CoV-2 (Becton Dickinson and Company, Maryland, USA, Ref: 256082) in accordance with manufacturer instructions. The BD Veritor™ system for Rapid Detection of SARS-CoV-2 is a chromatographic digital immunoassay intended for the direct and qualitative detection of SARS-CoV-2 nucleocapsid antigens in nasal swabs. In order to determine semi-quantitative differences in viral load between sample types, test band intensity was scored on a scale of 0-3, with 0 corresponding to a negative test and 3 corresponding to the maximum intensity observed.

### Section 2: Results and Discussion

Of the 44 subjects, 24 subjects tested negative and 9 subjects tested positive for SARS-CoV-2 antigens for all three sample types (anterior nares swabbing before nasal saline spraying, mid-turbinate swabbing, and anterior nares swabbing after nasal saline spraying). 20 subjects tested positive for SARS-CoV-2 antigens on at least one of the three sample types.

Table 10 summarizes the qualitative results (positive or negative) for these 20 subjects with each of the three sample types, and Table 11 summarizes the semi-quantitative positive test band intensities (0-3) for these 20 subjects with each of the three sample types (anterior nares swabbing before nasal saline spraying, mid-turbinate swabbing, and anterior nares swabbing after nasal saline spraying).

TABLE 10
Qualitative immunoassay results from 20 subjects who tested positive
for SARS-CoV-2 antigens on at least one of the three sample types

| | | MT | | |
|---|---|---|---|---|
| | | result | AN1 result | AN2 result |
| # | Pt ID | (P or N) | (P or N) | (P orN) |
| 1 | 2048 | P | N | P |
| 2 | 2060 | P | N | P |
| 3 | 2098 | P | P | P |
| 4 | 2093 | N | N | P |
| 5 | 2094 | P | N | N |
| 6 | 2042 | P | N | P |
| 7 | 2102 | P | N | P |
| 8 | 2075 | P | N | P |
| 9 | 2099 | P | N | P |
| 10 | 2126 | P | P | P |
| 11 | 2131 | P | N | P |
| 12 | 2119 | N | N | P |
| 13 | 2117 | N | P | N |
| 14 | 2121 | P | P | P |
| 15 | 2115 | P | P | P |
| 16 | 2116 | P | P | P |
| 17 | 2110 | P | P | P |
| 18 | 2149 | P | P | P |
| 19 | 2208 | P | P | P |
| 20 | 2219 | P | P | P |
| | Total | 17 Positive | 10 Positive | 18 Positive |

Table 10: MT = mid-turbinate swabbing, AN1 = anterior nares swabbing before nasal saline spraying, AN2 = anterior nares swabbing after nasal saline spraying.

7 out of the 11 subjects with discordant results (64%) tested positive for SARS-CoV-2 antigens both with mid-turbinate swabbing and anterior nares swabbing after nasal saline spraying, but negative with anterior nares swabbing before nasal saline spraying, indicating that nasal saline spraying increases anterior nares swab efficacy in viral material sampling and sensitivity in immunoassays.

Regarding the remaining 4 subjects with discordant results:
2 subjects tested positive for SARS-CoV-2 antigens with anterior nares swabbing after nasal saline spraying, but negative with mid-turbinate swabbing and anterior nares swabbing before nasal saline spraying;
1 subject tested positive for SARS-CoV-2 antigens with mid-turbinate swabbing, but negative with both anterior nares swabbings; and
1 subject tested positive for SARS-CoV-2 antigens with anterior nares swabbing before nasal saline spraying, but negative with both mid-turbinate swabbing and anterior nares swabbing after nasal saline spraying.

TABLE 11
Semi-quantitative immunoassay results from 20 subjects who tested
positive for SARS-CoV-2 antigens on at least one of the three sample types

| | | MT | AN1 | AN2 |
|---|---|---|---|---|
| | | band | band | band |
| # | Pt ID | intensity | intensity | intensity |
| 1 | 2048 | 1.5 | 0 | 2 |
| 2 | 2060 | 1.5 | 0 | 1 |
| 3 | 2098 | 1 | 0.5 | 3 |
| 4 | 2093 | 0 | 0 | 0.5 |
| 5 | 2094 | 0.1 | 0 | 0 |
| 6 | 2042 | 1.5 | 0 | 1 |
| 7 | 2102 | 0.5 | 0 | 0.1 |
| 8 | 2075 | 0.5 | 0 | 0.1 |
| 9 | 2099 | 0.5 | 0 | 1 |
| 10 | 2126 | 1.5 | 0.5 | 1 |
| 11 | 2131 | 1.5 | 0 | 0.5 |
| 12 | 2119 | 0 | 0 | 0.5 |
| 13 | 2117 | 0 | 0.1 | 0 |
| 14 | 2121 | 0.1 | 0.5 | 0.5 |
| 15 | 2115 | 2 | 0.1 | 1.5 |
| 16 | 2116 | 3 | 1 | 2.5 |
| 17 | 2110 | 2 | 0.1 | 2 |
| 18 | 2149 | 3 | 2 | 3 |
| 19 | 2208 | 2.5 | 2.5 | 3 |
| 20 | 2219 | 1 | 0.1 | 2.5 |
| | Average | 1.19 | 0.37 | 1.29 |

Table 11: Semi-quantitative results are subjective ratings of test band intensity from 0 (negative result) to 3 (highest positive test band intensity corresponding to the maximum intensity observed). MT = mid-turbinate swabbing, AN1 = anterior nares swabbing before nasal saline spraying, AN2 = anterior nares swabbing after nasal saline spraying.

Positive test band intensity for SARS-CoV-2 antigens was higher for anterior nares swabbing after nasal saline spraying than for anterior nares swabbing before nasal saline spraying in 8 of the 9 concordant positive results (i.e., concordant for all sample types) (89%), and equal for the remaining 1 concordant positive result (11%). In examining the relative immunoassay intensities of the two types of anterior nares swabbing (before and after nasal saline spraying) in subjects with both discordant and concordant results, 17 out of 20 subjects (85%) had stronger SARS-CoV-2 test band intensities from the anterior nares swab performed after nasal saline spraying compared to the paired anterior nares swab performed before nasal saline spraying.

The average test band intensity for SARS-CoV-2 antigens for mid-turbinate swabbing was 1.19 (95% CI: 0.73-1.64), for anterior nares swabbing before nasal saline spraying was 0.37 (95% CI: 0.043-0.70), and for anterior nares swabbing after nasal saline spraying was 1.29 (95% CI: 0.78-1.79). The difference in average test band intensity for SARS-CoV-2 antigens between anterior nares swabbing before nasal saline spraying and anterior nares swabbing after nasal saline spraying was statistically significant (p=0.000068). However, there was no statistically significant difference in the average test band intensity for SARS-CoV-2 antigens between anterior nares swabbing after nasal saline spraying and mid-turbinate swabbing (p=0.55). This indicates that nasal saline spraying improved sample quality and the resulting test sensitivity of anterior nares swabbing to a level similar to mid-turbinate swabbing.

In some applications, any of the techniques described in the report of Experiment 8 above are used in combination with the other techniques described herein.

In an embodiment, the techniques and apparatus described herein are combined with techniques and apparatus described in one or more of the following patent applications, which are assigned to the assignee of the present application and are incorporated herein by reference:
PCT Publication WO 2018/158768 to Fruchter et al.
US Patent Application Publication 2019/0381498 to Fruchter et al.
U.S. Provisional Application 62/727,208, filed Sep. 5, 2018
U.S. Provisional Application 62/727,268, filed Sep. 5, 2018
PCT Publication WO 2020/049566 to Fruchter et al.
PCT Publication WO 2020/049569 to Fruchter et al., and U.S. application Ser. No. 17/270,544 in the national stage thereof, which published as US Patent Application Publication 2021/0215585 to Fruchter et al.
US Patent Application Publication 2021/0102876 to Fruchter et al.
U.S. Provisional Application 62/896,295, filed Sep. 5, 2019
U.S. Provisional Application 62/988,145, filed Mar. 11, 2020
U.S. Provisional Application 62/988,259, filed Mar. 11, 2020
U.S. Provisional Application 63/020,723, filed May 6, 2020
U.S. Provisional Application 63/037,707, filed Jun. 11, 2020
U.S. Provisional Application 63/067,535, filed Aug. 19, 2020
U.S. Provisional Application 63/117,294, filed Nov. 23, 2020
U.S. Provisional Application 63/156,843, filed Mar. 4, 2021
U.S. Provisional Application 63/158,005, filed Mar. 8, 2021
U.S. Provisional Application 63/166,378, filed Mar. 26, 2021
U.S. Provisional Application 63/176,565, filed Apr. 19, 2021
PCT Publication WO 2021/044417 to Holtz et al.
PCT Publication WO 2021/181338 to Fruchter et al., and U.S. application Ser. No. 17/801,452 in the national stage thereof
PCT Publication WO 2021/181339 to Feldman et al., and U.S. application Ser. No. 17/802,614 in the national stage thereof
International Application PCT/IL2021/050519, filed May 6, 2021, which published as PCT Publication WO 2021/224925 to Levitz et al., and U.S. application Ser. No. 17/921,672 in the national stage thereof
U.S. Provisional Application 63/071,529, filed Aug. 28, 2020
PCT Publication WO 2022/044002 to Levitz et al.
U.S. Provisional Application 63/134,282, filed Jan. 6, 2021
PCT Publication WO 2022/149135 to Feldman et al.
U.S. Provisional Application 63/275,130, filed Nov. 3, 2021
U.S. Provisional Application 63/277,238, filed Nov. 9, 2021

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
intranasally dispensing nasal wash fluid into a nasal cavity of a subject;
thereafter, collecting a specimen sample by performing an anterior nares nasal swab; and
testing the specimen sample for the presence of a virus using a lateral flow immunoassay test strip.

2. The method according to claim 1, wherein collecting the specimen sample comprises performing the anterior nares swab without performing a nasopharyngeal swab.

3. The method according to claim 1, wherein collecting the specimen sample comprises performing the anterior nares swab without performing a mid-turbinate swab.

4. The method according to claim 1, wherein collecting the specimen sample comprises performing the anterior nares swab without performing a mid-turbinate swab and without performing a nasopharyngeal swab.

5. The method according to claim 1, wherein collecting the specimen sample comprises collecting between 25 and 150 microliters of material on a single swab.

6. The method according to claim 5, wherein the single swab comprises a flocked swab tip, and wherein collecting the specimen sample comprises collecting between 25 and 150 microliters of the material on the flocked swab tip.

7. The method according to claim 1, wherein intranasally dispensing the nasal wash fluid into the nasal cavity strengthens an intensity of a test band of the lateral flow immunoassay test strip when the virus is present in the anterior naris.

8. The method according to claim 1, wherein intranasally dispensing the nasal wash fluid into the nasal cavity, when the virus is present in the anterior naris, strengthens an intensity of a test band of the lateral flow immunoassay test strip compared to collecting the specimen sample without intranasally dispensing the nasal wash fluid into the nasal cavity.

9. The method according to claim 1, further comprising, after intranasally dispensing the nasal wash fluid and before collecting the specimen sample, sniffing, by the subject, the nasal wash fluid.

10. The method according to claim 1, further comprising waiting for a period of time between (a) concluding intranasally dispensing the nasal wash fluid into the nasal cavity and (b) collecting the specimen sample, the period of time at least 2 seconds and no more than 30 minutes.

11. The method according to claim 1, wherein intranasally dispensing the nasal wash fluid comprises intranasally dispensing the nasal wash fluid without blowing, by the subject, a nose of the subject immediately prior to intranasally dispensing the nasal wash fluid.

12. The method according to claim 1, wherein intranasally dispensing the nasal wash fluid comprises intranasally dispensing the nasal wash fluid into each of one or both nostrils of the subject in a total volume of between 0.15 and 0.5 mL per nostril.

13. The method according to claim 1, wherein intranasally dispensing the nasal wash fluid comprises intranasally dispensing the nasal wash fluid as one or more types of fluid discharge selected from the group consisting of: a spray, a mist, a pressurized aerosol, steam, and an atomized fluid.

14. The method according to claim 1, wherein the virus is Severe acute respiratory syndrome coronavirus 2 (SARS-CoV-2).

15. The method according to claim 1, wherein the virus is an Influenza virus.

16. A method comprising:
intranasally dispensing nasal wash fluid into a nasal cavity of a subject;
inserting a specimen sampler distally into the nasal cavity such that a farthest-inserted portion of the specimen sampler is positioned in an anterior naris of the nasal cavity;
collecting a specimen sample on the specimen sampler by contacting a wall of the anterior naris with the specimen sampler; and
testing the specimen sample for the presence of a virus using a lateral flow immunoassay test strip.

17. The method according to claim 16, wherein the virus is Severe acute respiratory syndrome coronavirus 2 (SARS-CoV-2).

18. The method according to claim 16, wherein the virus is an Influenza virus.

19. The method according to claim 16, wherein the specimen sampler comprises a head comprising an absorbent material, and wherein contacting the wall of the anterior naris with the specimen sampler comprises contacting the wall of the anterior naris with the absorbent material of the head of the specimen sampler.

20. The method according to claim 16, wherein the specimen sampler comprises a swab.

21. The method according to claim 16, wherein the specimen sampler comprises a brush.

22. The method according to claim 16, wherein the specimen sampler comprises a sponge.

23. The method according to claim 16, wherein the specimen sampler comprises a nasal plug.

24. The method according to claim 16, wherein the specimen sampler is shaped so as not to define any nozzles.

25. The method according to claim 16, wherein collecting the specimen sample comprises collecting the specimen sample on the specimen sampler without using suction.

26. The method according to claim 16, wherein collecting the specimen sample on the specimen sampler comprises collecting between 25 and 150 microliters of material on the specimen sampler.

27. The method according to claim 16, wherein intranasally dispensing the nasal wash fluid comprises intranasally dispensing the nasal wash fluid as one or more types of fluid discharge selected from the group consisting of: a spray, a mist, a pressurized aerosol, steam, and an atomized fluid.

28. The method according to claim 16, wherein inserting the specimen sampling into the nasal cavity comprises inserting the specimen sampling into the nasal cavity after intranasally dispensing the nasal wash fluid into the nasal cavity.

29. The method according to claim 16, wherein collecting the specimen sample on the specimen sampler by contacting the wall of the anterior naris with the specimen sampler comprises collecting the specimen sample on the specimen sampler by rubbing the wall of the anterior naris with the specimen sampler.

30. The method according to claim 29, wherein rubbing the wall of the anterior naris comprises rotating the specimen sampler while rubbing the wall of the anterior naris with the specimen sampler.
